SEQUENCE LISTING (9 sequences)

SEQ ID NO: 1 - protein, 115 aa, Artificial Sequence
  source: /note=“Description of Artificial Sequence Synthetic polypeptide”
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 2 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 3 - protein, 5 aa, Artificial Sequence
  source: /note=“Description of Artificial Sequence Synthetic peptide”
SYYWS

SEQ ID NO: 4 - protein, 16 aa, Artificial Sequence
  source: /note=“Description of Artificial Sequence Synthetic peptide”
HISYSGSANYNPSLKS

SEQ ID NO: 5 - protein, 7 aa, Artificial Sequence
  source: /note=“Description of Artificial Sequence Synthetic peptide”
WDDAFNI

SEQ ID NO: 6 - protein, 12 aa, Artificial Sequence
  source: /note=“Description of Artificial Sequence Synthetic peptide”
RASQSVSSSYLA

SEQ ID NO: 7 - protein, 7 aa, Artificial Sequence
  source: /note=“Description of Artificial Sequence Synthetic peptide”
GASSRAT

SEQ ID NO: 8 - protein, 9 aa, Artificial Sequence
  source: /note=“Description of Artificial Sequence Synthetic peptide”
QQYGSSPWT

SEQ ID NO: 9 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

(19) United States
(12) Patent Application Publication
(10) Pub. No.: US 2019/0292265 A1
(43) Pub. Date: Sep. 26, 2019

(54) Methods of Treating Crohn's Disease with an Anti-NKG2D Antibody

(71) Applicant: Janssen Biotech, Inc., Horsham, PA (US)

(72) Inventors: Carrie Brodmerkel, West Chester, PA (US); Mark Curran, Titusville, NJ (US); Linda Greenbaum, Florham Park, NJ (US); Shannon Telesco, Lansdale, PA (US)

(21) Appl. No.: 16/325,969

(22) PCT Filed: Aug. 17, 2017
(86) PCT No.: PCT/US17/47357

§ 371 Date: Feb. 15, 2019

Related U.S. Application Data
(60) Provisional application No. 62/377,358, filed on Aug. 19, 2016.

(51) Int. Cl. C07K 16/28; A61P 1/04; A61K 9/00; C12Q 1/6827

(52) U.S. Cl. CPC C07K 16/2851; C12Q 1/6827; A61K 9/0019; A61P 1/04

(57) ABSTRACT

The present invention is directed to methods for treating Crohn's Disease with an antibody that binds NKG2D. In particular, it relates to dosing regimens for administration of an anti-NGK2D antibody. It also relates to methods of selecting patients for treatment with an anti-NKG2D antibody.

### CROSS-REFERENCE TO RELATED APPLICATIONS

This application is a 371 national stage application of International Application Number PCT/US2017/047357, filed 17 Aug. 2017, which claims priority to U.S. Provisional Application Ser. No. 62/377358, filed 19 Aug. 2016. The entire contents of each of the aforegoing applications is incorporated herein by reference in their entireties.

### SEQUENCE LISTING

This application contains a Sequence Listing which has been submitted electronically in ASCII format and is hereby incorporated by reference in its entirety. Said ASCII copy, created on 8 Feb. 2019, is named JBI5096USNATLSEQLIST and is 6086 bytes in size.

### FIELD OF THE INVENTION

The present invention is directed to methods for treating Crohn's Disease with an antibody that binds NKG2D. In particular, it relates to dosing regimens for administration of an anti-NGK2D antibody. It also relates to methods of selecting patients for treatment with an anti-NKG2D antibody.

### BACKGROUND OF THE INVENTION

Crohn's disease (CD) is a chronic irritable bowel disease characterized by uncontrolled immune responses (Baumgart D C, Sandborn W J. Lancet 2012; 380:1590-605; Mayer L. J Gastroenterol 2010; 45:9-16.). Therapy for CD is based on suppression of the immune system by blockade of inflammatory processes with immune suppressants or biologic therapies. Progress has been considerable over the last decade, mainly due to the development and extensive usage of anti-tumour necrosis factor (TNF) monoclonal antibodies. Despite initial efficacy, long-term benefit is observed in less than half of patients with CD treated with anti-TNF antibodies. (Allez M, Vermeire S, Mozziconacci N, et al. antibodies. Aliment Pharmacol Ther 2010; 31:92-101.). Biologics with new targets have been developed, including monoclonal antibodies targeting the trafficking of immune cells, yet therapies with novel mechanisms of action are still required.

The persistence of intestinal inflammatory lesions in CD is mediated by an active crosstalk between immune and non-immune cells, and T cells are key players in this pathogenic process (Allez M, Mayer L. Regulatory T cells: Peace keepers in the gut. Inflamm Bowel Dis 2004; 10:666-76.). The inflamed mucosa is heavily infiltrated with activated T lymphocytes, which produce inflammatory cytokines, exhibit cytotoxic properties and contribute to mucosal damage (Croitoru K, Zhou P. T-cell-induced mucosal damage in the intestine. Curr Opin Gastroenterol 2004; 20:581-6.; Neurath M F, Finotto S, Fuss I, et al. Trends Immunol 2001; 22:21-6.). The accumulation of these immune cells relies on an active recruitment from the bloodstream, a sustained cell cycling and diminished susceptibility of cells to undergo apoptosis. T-cell activation relies on the recognition of specific antigens by the T-cell receptor and the concomitant delivery of a costimulatory signal. Interestingly, mucosal T cells may express innate receptors that provide this costimulatory signal. Natural killer group 2 member D (NKG2D) is an activating receptor present on the surface of natural killer (NK) cells, some NK T cells, CD8+ cytotoxic T cells, gammadelta T cells and CD4+ T cells, under certain conditions. (Champsaur M, Lanier L L. Immunol Rev 2010; 235:267-85.; Jamieson A M, Diefenbach A, McMahon C W, et al. Immunity 2002; 17:19-29.).

The ligands that bind to human NKG2D are major histocompatibility complex class I-related molecules A and B and UL-16-binding proteins, all of which have increased expression with cellular stress. (Groh V, Bahram S, Bauer S, et al. Proc Natl Acad Sci USA 1996; 93:12445-50.). A number of these NKG2D ligands are expressed on epithelial cells and are unregulated in the inflamed mucosa in IBD (Allez M, Tieng V, Nakazawa A, et al. Gastroenterology 2007; 132:2346-58; La Scaleia R, Stoppacciaro A, Oliva S, et al. Inflamm Bowel Dis 2012; 18:1910-22; Tieng V, Le Bouguenec C, du Merle L, et al. Proc Natl Acad Sci USA 2002; 99:2977-82.). Thus, the intestinal epithelium may modulate a variety of T-cell responses through direct interactions via the NKG2D pathway (Allez M, Mayer L. Inflamm Bowel Dis 2004; 10:666-76; h V, Bahram S, Bauer S, et al. Proc Natl Acad Sci USA 1996; 93:12445-50.).

An increased expression of NKG2D on CD4+ T cells is observed in CD (Allez M, Tieng V, Nakazawa A, et al. Gastroenterology 2007; 132:2346-58.). CD4+NKG2D+T cells exhibit specific cytotoxic activity and are able ex vivo to kill target cells expressing NKG2D ligands and are also an important source of inflammatory cytokines (e.g., TNFα, interferon (IFN)γ and interleukin-17 (IL-17)) (Allez M, Tieng V, Nakazawa A, et al. Gastroenterology 2007; 132:2346-58; Pariente B, Mocan I, Camus M, et al. Gastroenterology 2011; 141:217-26, 226.e1-2.). The production of these cytokines is strongly enhanced ex vivo by costimulation of the T-cell receptor and the NKG2D receptor (Allez M, Tieng V, Nakazawa A, et al. Gastroenterology 2007; 132:2346-58; Pariente B, Mocan I, Camus M, et al. Gastroenterology 2011; 141:217-26, 226.e1-2.). Interestingly, most of the T-cell oligoclonal expansions found in the inflamed mucosa of patients with CD correspond to CD4+ T cells expressing NKG2D (Camus M, Esses S, Pariente B, et al. Immunol 2014; 7:325-34.). The implication of CD4+NKG2D+ T cells in gut inflammation has been further demonstrated in a murine model of transferinduced colitis (Kjellev S, Haase C, Lundsgaard D, et al. Eur J Immunol 2007; 37:1397-406; Ito Y, Kanai T, Totsuka T, et al. Am J Physiol Gastrointest Liver Physiol 2008; 294:G199-207.). Administration of a specific NKG2D-blocking antibody decreased NKG2D expression on CD4+ cells and attenuated the development of colitis. NKG2D may also modulate the function of other T-cell subsets including CD8+ T cells and NK cells, particularly cytotoxicity, as shown in coeliac disease (Hüe S, Mention J J, Monteiro R C, et al. Immunity 2004; 21:367-77; Meresse B, Chen Z, Ciszewski C, et al. Immunity 2004; 21:357-66.). These data support the potential role of the NKG2D pathway in the overactivation of effector T cells in CD.

Therefore, a need exists in the art for effective dosing regimens for the treatment of a subject with Crohn's disease with an anti-NKG2D antibody as well as methods of selecting patients in whom an anti-NKG2D antibody will show clinical efficacy. The invention herein provides such methods.

### SUMMARY OF THE INVENTION

This application provides methods of treating a subject suffering from Crohn's disease, the methods comprising administering to the human patient a safe and effective amount of an anti-NKG2D antibody comprising CDR1, CDR2 and CDR3 domains of the heavy chain variable region having the sequences set forth in SEQ ID NO: 3,4 and 5, respectively and CDR1, CDR2 and CDR3 domains of the light chain variable region having the sequences set forth in SEQ ID NO: 6,7, and 8.

In some embodiments, the anti-NKG2D antibody is administered in at least one administration cycle, wherein for each of the at least one administration cycle, the anti-NKG2D antibody is administered as follows: (a) one dose of 400 mg anti-NKG2D antibody and (b) at least one dose of 200 mg anti-NKG2D antibody.

In some embodiments the anti-NKG2D antibody is formulated for intravenous or subcutaneous administration.

In some embodiments, the anti-NKG2D treatment consists of up to 6 cycles.

In some embodiments, a 200 mg dose of an anti-NKG2D antibody is administered eleven times.

In some embodiments described herein the periodic administration of the NKG2D antibody is once every 2 weeks for 22 weeks after administration of an initial dose.

In some embodiments, the amount of the anti-NKG2D antibody is effective to reduce a symptom of Crohn's disease in the subject, induce clinical response, induce or maintain clinical remission, inhibit disease progression, or inhibit a disease complication in the subject.

In some embodiments the amount of the anti-NKG2D antibody is effective to reduce the Crohn's Disease Activity Index score of the subject, lower the C-Reactive Protein level of the subject, lower the fecal calprotein level of the subject, or reduce the number of open draining fistulas in the subject.

In another embodiment the subject tested positive for single nucleotide polymorphisms (SNPs) rs2255336 and rs2239705 prior to the administration of the anti-NKG2D antibody.

In a preferred embodiment the anti-NKG2D antibody comprises a heavy-chain variable region comprising SEQ ID NO: 1 and a light-chain variable region comprising SEQ ID NO: 2.

Another aspect of the invention relates to method of treating a human patient with Crohn's disease, the method comprising the steps of:

(a) determining whether the human patient has a SNP in an NKG2D receptor gene or MICB gene by obtaining a biological sample from the human patient and performing a genotyping assay on the biological sample;

(b) administering an anti-NKG2D receptor antibody if the patient has the SNP, wherein the anti-NKG2D antibody comprises CDR1, CDR2 and CDR3 domains of the heavy chain variable region having the sequences set forth in SEQ ID NO: 3, 4 and 5, respectively and CDR1, CDR2 and CDR3 domains of the light chain variable region having the sequences set forth in SEQ ID NO: 6, 7, and 8, respectively.

### BRIEF DESCRIPTION OF THE DRAWINGS

FIG. 1: Schematic representation of the anti-NKG2D clinical study.

FIG. 2: Schematic Overview of Part I of the anti-NKG2D clinical study. (*) indicates timepoint for primary endpoint, (**) indicated final efficacy and safety visit, (†) Placebo nonresponders receive high dose: 400 mg at Week 12 and 200mg at Weeks 14-22.

FIG. 3: Schematic Overview of Part II of the anti-NKG2D clinical study. (*) indicates timepoint for primary endpoint, (**) indicated final efficacy and safety visit, (†) Placebo nonresponders receive middle dose: 150 mg at Week 12, 75 mg at Weeks 14, 16, and 20, a) Bio-IR (intolerant/refractory) will be randomized 1:1:1:1:1 ratio.

FIG. 4: Graph representing the change in CDAI score in anti-NKG2D treated patients based upon the genotype for the MICB-rs2239705 and NKG2D-rs2255336 SNP.

### DETAILED DESCRIPTION OF THE PREFERRED EMBODIMENTS

### Definitions:

As used herein, “hNKG2D” and, unless otherwise stated or contradicted by context, the terms “NKG2D,” also known as “NKG2-D,” “CD314,” “D1252489E,” “KLRK1,” “killer cell lectin-like receptor subfamily K, member 1,” and “KLRK1,” refer to a human killer cell activating receptor gene, its mRNA (e.g., NCBI RefSeq NM 007360), and its gene product (NCBI RefSeq NP 031386 shown as SEQ ID NO:9), or naturally occurring variants thereof. In NK and T cells, the ligand-binding form of the hNKG2D receptor is a homodimer (Li et al, Nat Immunol 2001; 2:443-451). The hNKG2D receptor is typically presented at the surface in complex with DAP10 (Wu et al, J Exp Med 2000; 192:1059 et seq.; NCBI Accession No. AAG29425, AAD50293) and has been suggested to also form higher order complexes. Any activity attributed herein to hNKG2D, e.g., cell activation, antibody recognition, etc., can also be attributed to hNKG2D in the form of a complex or higher-order complexes with DAP10, and/or other components.

Human NKG2D
(SEQ ID NO: 9)
MGWIRGRRSRHSWEMSEFHNYNLDLKKSDFSTRWQKQRCPVVKSKCRENA
SPFFFCCFIAVAMGIRFIIMVTIWSAVFLNSLFNQEVQIPLTESYCGPCP
KNWICYKNNCYQFFDESKNWYESQASCMSQNASLLKVYSKEDQDLLKLVK
SYHWMGLVHIPTNGSWQWEDGSILSPNLLTIIEMQKGDCALYASSFKGYI
ENCSTPNTYICMQRTV

The term “antibody” herein is used in the broadest sense and specifically includes full-length monoclonal antibodies, polyclonal antibodies, and, unless otherwise stated or contradicted by context, antigen-binding fragments, antibody variants, and multispecific molecules thereof, so long as they exhibit the desired biological activity. Generally, a full-length antibody is a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. General principles of antibody molecule structure and various techniques relevant to the production of antibodies are provided in, e.g., Harlow and Lane, ANTIBODIES: A LABORATORY MANUAL, Cold Spring Harbor Laboratory Press, Cold Spring Harbor, N.Y., (1988).

An “antigen-binding fragment” of an antibody is a molecule that comprises a portion of a full-length antibody which is capable of detectably binding to the antigen, typically comprising one or more portions of at least the VH region. Antigen-binding fragments include multivalent molecules comprising one, two, three, or more antigen-binding portions of an antibody, and single-chain constructs wherein the VL and VH regions, or selected portions thereof, are joined by synthetic linkers or by recombinant methods to form a functional, antigen-binding molecule. While some antigen-binding fragments of an antibody can be obtained by actual fragmentation of a larger antibody molecule (e.g., enzymatic cleavage), most are typically produced by recombinant techniques.

The terms “antibody derivative” and “immunoconjugate” are used interchangeably herein to denote molecules comprising a full-length antibody or an antigen-binding fragment thereof, wherein one or more amino acids are chemically modified, e.g., by alkylation, PEGylation, acylation, ester formation or amide formation or the like, e.g., for linking the antibody to a second molecule. Exemplary modifications include PEGylation (e.g., cysteine-PEGylation), biotinylation, radiolabelling, and conjugation with a second agent (such as a cytotoxic agent).

A “multispecific molecule” comprises an antibody, or an antigen-binding fragment thereof, which is associated with or linked to at least one other functional molecule (e.g. another peptide or protein such as another antibody or ligand for a receptor) thereby forming a molecule that binds to at least two different binding sites or target molecules. Exemplary multispecific molecules include bi-specific antibodies and antibodies linked to soluble receptor fragments or ligands.

The term “human antibody”, as used herein, is intended to include antibodies having variable regions in which both the framework and CDR regions are derived from (i.e., are identical or essentially identical to) human germline immunoglobulin sequences. Furthermore, if the antibody contains a constant region, the constant region also is “derived from” human germline immunoglobulin sequences. The human antibodies of the invention may include amino acid residues not encoded by human germline immunoglobulin sequences (e.g., mutations introduced by random or site-specific mutagenesis in vitro or by somatic mutation in viva). However, the term “human antibody”, as used herein, is not intended to include anti-bodies in which CDR sequences derived from the germline of another mammalian species, such as a mouse, have been grafted onto human framework sequences.

A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992), WO 92/02190, US Patent Application 20060073137, and U.S. Pat. Nos. 6,750,325, 6,632,927, 6,639,055, 6,548,640, 6,407,213, 6,180,370, 6,054,297, 5,929,212, 5,895,205, 5,886,152, 5,877,293, 5,869,619, 5,821,337, 5,821,123, 5,770,196, 5,777,085, 5,766,886, 5,714,350, 5,693,762, 5,693,761, 5,530,101, 5,585,089, and 5,225,539.

The term “hypervariable region” when used herein refers to the amino acid residues of an antibody that are responsible for antigen binding. The hypervariable region generally comprises amino acid residues from a “complementarity-determining region” or “CDR” (residues 24-34 (L1), 50-56 (L2) and 89-97 (L3) in the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. “Framework region” or “FR” residues are those VH or VL residues other than the CDRs as herein defined.

An “epitope” or “binding site” is an area or region on an antigen to which an antigen-binding peptide (such as an antibody) specifically binds. A protein epitope may comprise amino acid residues directly involved in the binding (also called the immunodominant component of the epitope) and other amino acid residues, which are not directly involved in the binding, such as amino acid residues which are effectively blocked by the specifically antigen binding peptide (in other words, the amino acid residue is within the “solvent-excluded surface” and/or “footprint” of the specifically antigen binding peptide). The term epitope herein includes both types of amino acid binding sites in any particular region of a hNKG2D that specifically binds to an anti-hNKG2D antibody, or another hNKG2D-specific agent according to the invention, unless otherwise stated (e.g., in some contexts the invention relates to anti-bodies that bind directly to particular amino acid residues). NKG2Ds may comprise a number of different epitopes, which may include, without limitation, (1) linear peptide antigenic determinants, (2) conformational antigenic determinants which consist of one or more non-contiguous amino acids located near each other in a mature NKG2D conformation; and (3) post-translational antigenic determinants which consist, either in whole or part, of molecular structures covalently attached to a NKG2D, such as carbohydrate groups. Unless otherwise specified or contradicted by context, conformational antigenic determinants comprise NKG2D amino acid residues within about 4 A distance from an atom of an antigen-binding peptide.

The phrase “binds to essentially the same epitope or determinant as” an antibody of interest (e.g., MS or 21F2) means that an antibody “competes” with the antibody of interest for NKG2D molecules to which the antibody of interest specifically binds.

A “paratope” is an area or region of an antigen-binding portion of an antibody that specifically binds an antigen. Unless otherwise stated or clearly contradicted by context, a paratope may comprise amino acid residues directly involved in epitope binding, several of which are typically in CDRs, and other amino acid residues, which are not directly involved in the binding, such as amino acid residues which are effectively blocked by the specifically bound antigen (in other words, the amino acid residue is within the “solvent-excluded surface” and/or “footprint” of the specifically bound antigen).

The ability of an anti-NKG2D antibody to “block” the binding of a NKG2D molecule to a natural NKG2D-ligand (e.g., MICA), means that the antibody, in an assay using soluble or cell-surface associated NKG2D and ligand molecules, can detectably reduce the binding of a NKG2D-molecule to the ligand in a dose-dependent fashion, where the NKG2D molecule detectably binds to the ligand in the absence of the antibody. An exemplary assay for determining whether an anti-NKG2D antibody is capable of blocking MICA-binding is provided in Example 3. The same assay can be used for testing antibody-mediated blocking of other NKG2D ligands.

A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence and/or additions at one or both termini.

The term “substantially identical” in the context of two amino acid sequences means that the sequences, when optimally aligned, such as by the programs GAP or BESTFIT using default gap weights, share at least about 50 percent sequence identity. Typically sequences that are substantially identical will exhibit at least about 60, at least about 70, at least about 80, at least about 90, at least about 95, at least about 98, or at least about 99 percent sequence identity.

“Corresponding” amino acid positions in two substantially identical amino acid sequences are those aligned by any of the protein analysis software referred to herein.

A nucleic acid sequence (or element) is “operably linked” to another nucleic acid sequence (or element) when it is placed into a functional relationship with the other nucleic acid sequence. For example, DNA for a pre-sequence or secretory leader is operably linked to DNA for (i.e., coding for expression of) a polypeptide if it is expressed as a pre-protein that participates in the secretion of the polypeptide; a promoter or enhancer is operably linked to a coding sequence if it affects the transcription of the sequence; or a ribosome-binding site is operably linked to a coding sequence if it is positioned so as to facilitate translation. Generally, “operably linked” means that the DNA sequences being linked are contiguous, and, in the case of a secretory leader, contiguous and in reading phase. However, some elements, such as enhancers, do not have to be contiguous with a coding sequence in order to be operably linked. Linking typically is accomplished by ligation at convenient restriction sites. If such sites do not exist, the synthetic oligonucleotide adaptors or linkers may be used in accordance with conventional practice.

An “isolated” molecule is a molecule that is the predominant species in the composition wherein It is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of an antibody molecule will exhibit 98%, 98%, or 99% homogeneity for antibody molecules in the context of all present peptide species in the composition or at least with respect to substantially active peptide species in the context of proposed use.

The terms “treating”, and “treatment” and the like are used herein to generally mean obtaining a desired pharmacological, physiological or therapeutic effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having (b) inhibiting the disease, i.e., arresting its development; or (c) relieving the disease, i.e., causing regression of the disease and/or its symptoms or conditions. The invention is directed towards treating a patient's suffering from disease related to pathological inflammation. The present invention is involved in preventing, inhibiting, or relieving adverse effects attributed to pathological inflammation over long periods of time and/or are such caused by the physiological responses to inappropriate inflammation present in a biological system over long periods of time,

In one aspect, the present invention provides methods of treating a subject. The method can, for example, have a generally salubrious effect on the subject, e.g., it can increase the subject's expected longevity. Alternatively, the method can, for example, treat, prevent, cure, relieve, or ameliorate (“treat”) a disease, disorder, condition, or illness (“a condition”). In one embodiment, the present invention provides a method of treating a condition in a subject comprising administering the pharmaceutical composition comprising an specific antibody to the subject, wherein the condition is treatable by reducing the activity (partially or fully) of NKG2D in the subject, Treating encompasses both therapeutic administration administration when signs and symptoms of the disease or condition are apparent) as well prophylactic or maintenance therapy (i.e., administration when the disease or condition is quiescent), as well as treating to induce remission and/or maintain remission. Accordingly, the severity of the disease or condition can be reduced (partially, significantly or completely), or the signs and symptoms can be prevented or delayed (delayed onset, prolonged remission, or quiescence).

Among the conditions to be treated in accordance with the present invention are conditions in which NKG2D is associated with or plays a role in contributing to the underlying disease or disorder or otherwise contributes to a negative symptom. Such conditions include Crohn's Disease.

As used herein, a “single nucleotide polymorphism” or “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals. As used herein, “haplotype” refers to a group of genes, variations in DNA or set of SNPs that tend to be inherited together. It can also refer to a combination of alleles or to a set of SNPs found on the same chromosome.

As used herein, a “safe and effective amount of an anti-NKG2D antibody” means the amount of an anti-NKG2D antibody that is effective to treat Crohn's disease or a symptom associated therewith without causing unacceptable drug related adverse events, when administered to a subject.

The term “efficacy” as used herein in the context of a dosage regimen refers to the effectiveness of a particular treatment regimen. Efficacy can be measured based on change the course of the disease in response to an agent of the present invention in one embodiment, an antigen binding protein (for example, an anti-NKG2D antibody) is administered to the subject in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question. The degree of improvement generally is determined by a physician, who may make this determination based on signs, symptoms, biopsies, or other test results, and who may also employ questionnaires that are administered to the subject, such as quality-of-life questionnaires developed for a given disease.

The NKG2D-specific antibody may be administered to achieve an improvement in a subject's condition. Improvement may be indicated by a decrease in an index of disease activity, by amelioration of clinical symptoms or by any other measure of disease activity. One such index of disease is the Crohn's Disease Activity Index (CDAI). The index consists of eight factors, each summed after adjustment with a weighting factor. The components of the CDAI and weighting factors are the following:

| | Weighting |
|---|---|
| Clinical or laboratory variable | factor |
| Number of liquid or soft stools each day for seven days | x 2 |
| Abdominal pain (graded from 0-3 on severity) each day | x 5 |
| for seven days | |
| General well-being, subjectively assessed from 0 | x 7 |
| (well) to 4 (terrible) each day for seven days | |
| Presence of complications* | x 20 |
| Taking Lomotil or opiates for diarrhea | x 30 |
| Presence of an abdominal mass (0 as none, 2 as | x 10 |
| questionable, 5 as definite) | |
| Hematocrit of <0.47 in men and <0.42 in women | x 6 |
| Percentage deviation from standard weight | x 1 |

Clinical Remission of Crohn's disease is defined when a CDAI score is less than 150.

### Anti-NKG2D Antibodies

The antibodies of the invention are characterized by particular functional and/or structural features or properties. Assays to evaluate the functional activities of anti-hNKG2D antibodies are described in detail in U.S. Pat. No. 7,879,985 incorporated herein by reference, and structural properties such as, e.g., amino acid sequences, are also described in U.S. Pat. No. 7,879,985 incorporated herein by reference.

### Functional Properties

The antibodies of the invention bind to hNKG2D. In one embodiment, an antibody of the invention binds to hNKG2D with high affinity, for example with a KD of 10 ⁷ M or less, a KD of 10⁻⁸ M or less, a KD of 1 nM or less, a KD of 0.3 nM or less, a KD of 0.2 nM or less, 0.1 nM or less, 0.05 nM or less, or 0.01 nM or less. In a particular embodiment, the antibody binds to hNKG2D with an affinity of 0.1 nM or less.

In one aspect, the invention provides antibodies that also bind to one or more NKG2D orthologs in a monkey such as a cynomolgous monkey (Macaca fascicularis, NCBI accession No. A.1426429) and a rhesus monkey (Macaca mulatta, NCBI accession No. M554302), and/or to a 11NKG2D homodimer, correctly folded monomeric full-length hNKG2D, hNKG2D fragment comprising an extra cellular portion of hNKG2D, denatured hNKG2D, or to any combination of the preceding NKG2D forms. For example, as demonstrated in Example 5 of U.S. Pat. No. 7,987,985, the binding of human antibodies 21F2 and MS to specific cynomolgous cell types were more than about 65% and about 75%, respectively, of their binding to the same human cell types, per the corresponding EC50 (i.e., the half maximal effective concentration) values. Accordingly, in one embodiment, an antibody of the invention binds to cynomolgous and/or rhesus NKG2D with similar affinity or efficacy as it binds to hNKG2D. For example, an antibody can bind to NKG2D-expressing cynomolgous or rhesus NK or T cells with an EC50 of about 50% or more, about 65% or more, or about 75% or more, of the corresponding EC50 for a corresponding population of NKG2D-expressing human NK or T cells. Additionally or alternatively, an antibody can bind to cynomolgous or rhesus NKG2D with an affinity of about 30% or more, about 50% or more, about 65% or more, or about 75% or more, about 80% or more, about 85% or more, or about 90% or more, of the affinity for hNKG2D. Such antibodies have the advantage of allowing for toxicity testing in the most suitable animal model (or models) prior to use in humans.

In one particular aspect, antibodies of the invention also bind a form of NKG2D that known murine anti-hNKG2D antibodies such as ON72 do not bind. Specifically, as described in Example 3 of U.S. Pat. No. 7,987,985, pre-incubation with ON72 only blocked about 82% of subsequently added human 16F16 antibody from binding to hNKG2D, while pre-incubation with 16F16 blocked about 95% of subsequently added ON72 from binding to hNKG2D.

Furthermore, the antibodies of the invention can reduce or inhibit hNKG2D-mediated activation of NK or T cells, i.e., antagonize the hNKG2D receptor. This may be tested in, e.g., one or more cytotoxicity assays described herein or known in the art. For example, an antibody inhibits hNKG2D-mediated activation of an NK or T cell if it inhibits the NK- or T cell-mediated killing of an NKG2D-ligand-expressing target cell by at least 10%, more preferably by at least 30%, even more preferably by at least 40%, at least 50%, at least 60%, at least 70%, at least 80% or at least 90%, as compared to target cell killing in the absence of any anti-hNKG2D antibody or in the presence of a non-specific, control antibody.

Antibodies of the invention that are hNKG2D antagonists can have no or low agonist activity. Preferably, such antibodies are human or humanized. Agonist activity may be tested in one of the assay described herein, or an assay known in the art. For example, one type of assay is a co-stimulation assay measuring proliferation of peripheral blood lymphocytes (PBMCs) stimulated with low levels of CD3 in the presence or absence of immobilized anti-NKG2D antibody (see Example 10 of U.S. Pat. No. 7,987,985). In such an assay, proliferation in the presence of an antibody of the invention is not more than 30%, not more than 20%, not more than 10%, not more than 5% or not significantly higher than in the absence of antibody. Preferably, proliferation in the presence of an antibody of the invention is not significantly higher than in the absence of antibody. In an additional or alternative embodiment, hNKG2D agonist activity of an antibody of the invention in an agonist assay is not more than 30%, not more than 20%, not more than 10%, not more than 5%, or not significantly higher than a control value. The control is preferably a negative control, such as, e.g., in the absence of antibody, in the absence of cell or another reagent, and/or in the presence of an irrelevant antibody. Preferably, agonist activity of an antibody of the invention is not significantly higher than a control value.

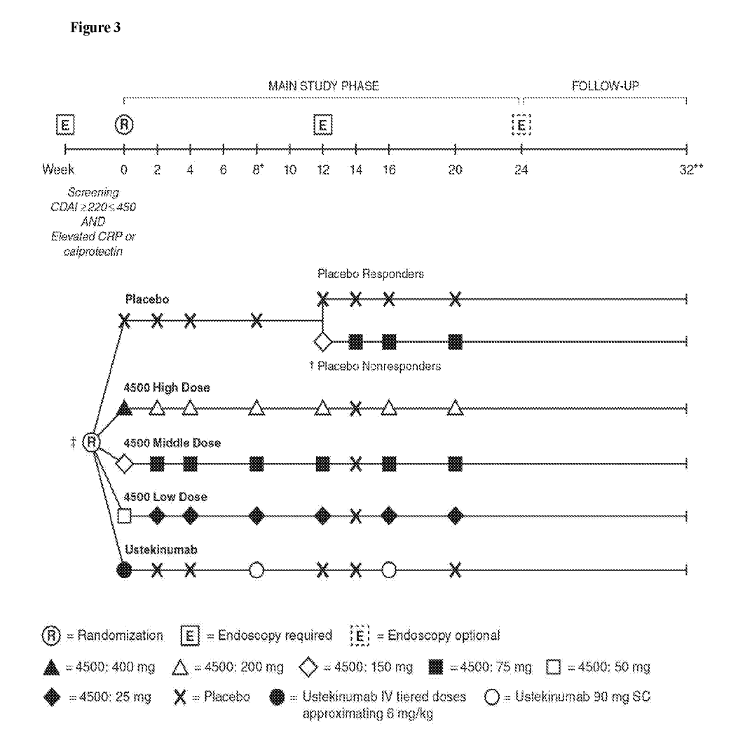

In another aspect, the invention provides antibodies that have a lower, preferably substantially lower, EC50 concentration for blocking ligand-induced cytotoxicity than for binding to cell-surface NKG2D of an NK or T cell. For example, for ON72, the EC50 concentration for binding to cell-surface NKG2D expressed on BaF/3 cells (0.062 μg/ml) was similar to the EC50 concentration for blocking NK-cell mediated killing of ligand-(ULBP3-) expressing target cells (0.065 μg/ml), whereas 21F2 had a lower, and MS a substantially lower, EC50 for blocking cytotoxicity (21F2: 0.021 μg/ml; MS: 0.012 μg/ml) than for binding to cell-surface NKG2D (21F2: 0.033 μg/ml; MS: 0.032 g/ml) (see Examples 6 and 9 of U.S. Pat. No. 7,987,985). Further, MS achieved maximum blocking of cytotoxicity at lower concentrations (a concentration corresponding only to about 80% saturation of cell-associated NKG2D-receptors, FIG. 3 of U.S. Pat. No. 7,987,985) than 21F2 and 16F16 (which had concentrations corresponding to saturating concentrations or higher, FIG. 3 of U.S. Pat. No. 7,987,985). Thus, in one embodiment, the invention provides antibodies, preferably human or humanized antibodies, that have a lower EC50 concentration for blocking ligand-induced cytotoxicity than for binding to cell-surface NKG2D of an NK or T cell. The EC50 for blocking cytotoxicity of NK or T cells of a cell line or other suitable preparation can be, e.g., about 95% or less, about 90% or less, about 85% or less, about 80% or less, about 70% or less, about 50% or less, or about 40% or less, of the EC50 for binding to cell-surface NKG2D of the same cell line or preparation. Exemplary cell lines for testing include NK-92 and NKL cells.

In another embodiment, the invention provides antibodies that achieve maximum blockage of NK cell cytotoxicity at a concentration lower than the concentration required to saturate the available hNKG2D-receptors. In a specific embodiment, the antibodies also compete with MS in binding to hNKG2D. In another specific embodiment, such antibodies bind to essentially the same hNKG2D epitope as MS.

The antibodies may reduce or inhibit NKG2D-mediated activation by, e.g., interfering with the hNKG2D-binding of one or more endogenous hNKG2D-ligands. For example, the antibodies may reduce or inhibit the hNKG2D-binding of MICA; MIICB; ULBP1; ULBP2; ULBP4; and/or RAET1-family member; e.g., by reducing or inhibiting the hNKG2D-binding of MICA; or of MICA and MICB, or of MICA and ULBP3; or of MICA, MICB, and ULBP3; or of MICA, MICR, and all ULBP1, -2, -3, and 4; or of MICA, MICB, and one or more RAET1 family members. The ability of an antibody to inhibit hNKG2D-binding of endogenous NKG2D-ligands can be evaluated using binding or competition assays described herein. In one embodiment, antibodies of the invention are capable of inhibiting at least 30% of ligand binding, or at least 50% of ligand binding, or at least 70% of ligand binding, or at least 80%, or at least 90% of ligand binding. In another embodiment, the IC50 for an antibody of the invention to inhibit the hNKG2D-binding of 1 μg MICA-rnFc is 1 nM or less, 0.5 nM or less, 0.2 nM or less, 0.1 nM or less, 0.05 nM or less, or 0.02 nM or less, 0.01 nM or less, 0,005 or less, or 0.002 or less. In another embodiment, full blockage of 1 μg MICA-mFc binding is achieved at an antibody concentration of 5 nM or less, 1 nM or less. 0.7 nM or less, 0.5 nM or less, or 0.2 nM or less, 0.1 nM or less, 0.05 nM or less, or about 0.02 nM or less. In one embodiment, the invention provides antibodies, especially human antibodies, that are as efficient or more efficient in reducing or inhibiting ligand hNKG2D-binding, such as, e.g., MICA binding to hNKG2D, than any of ON72, BAT221, 5C6, 1D11, ECM217, and 149810.

Additionally or alternatively, an anti-hNKG2D antibody of the invention can be capable of reducing the amount of cell-surface hNKG2D upon (i.e., following) binding. Reduction of cell-surface associated hNKG2D upon binding of an antibody can be an advantageous feature, since it reduces the number of hNKG2D receptors available for ligand binding and subsequent activation. Without being limited to theory, this reduction may be caused by NKG2D down-modulation, internalization, or other mechanism. As described in U.S. Pat. No. 7,987,985, anti-hNKG2D antibodies having a human Fe-region, such as human antibodies, are capable of effectively reducing the amount of cell-surface hNKG2D. For example, human anti-hNKG2D antibodies 16F16, MS, and 21F2 all reduced the amount of cell-surface hNKG2D with about 75% or more after overnight incubation in the absence of serum, with MS being the most effective, achieving 75-90% downmodulation at a low concentration (FIGS. 15-17 of U.S. Pat. No. 7,987,985). Also, in the presence of serum, an MS concentration corresponding to less than saturating concentration on hNKG2D-expressing BaF/3 cells achieved maximum downmodulation (FIG. 16B of U.S. Pat. No. 7,987,985). Accordingly, in one embodiment, the invention provides antibodies binding to hNKG2D that are able to achieve maximum down-modulation of hNKG2D at less than saturating concentrations. In another embodiment, such antibodies also compete with MS in binding to hNKG2D. In another embodiment, such antibodies also bind to essentially the same hNKG2D epitope as MS. An antibody of the invention can be capable of reducing cell surface hNKG2D by at least 10%, at least 20%, at least 30%, at least 50%, at least 70%, or at least 90% as compared to cell-surface hNKG2D in the absence of anti-hNKG2D antibody or in the presence of a non-specific control antibody. Preferably, the antibodies achieve reduction of cell-surface NKG2D while causing no or minimal activation of NKG2D-receptor signalling, i.e., with no or minimal agonist activity. Exemplary assays for evaluating cell surface hNKG2D and agonistic activity of anti-hNKG2D antibodies are described herein. In one embodiment, the invention provides antibodies, particularly human antibodies, which are capable of a higher degree of down-modulation than a control antibody selected from ON72, BAT221, 5C6, 1D11, ECM217, and 149810. In another embodiment, an anti-hNKG2D antibody of the invention can be capable of achieving maximum down-modulation of cell-surface NKG2D expressed by a cell or cell-line at a concentration lower than a saturating concentration.

In another embodiment, the invention provides antibodies that compete with and/or bind to the same epitope on hNKG2D as 16F16, 16F31, MS. and/or 21F2, more preferably MS and/or 21F2. Such antibodies can be identified based on their ability to cross-compete with 16F16, 16F31, MS, or 21F2 in standard hNKG2D binding assays as described herein. The ability of a test antibody to inhibit the binding of 16F16, 16F31, MS, or 21F2 to hNKG2D demonstrates that the test antibody can compete with 16F16, 16F31, MS, or 21F2 for binding to hNKG2D and thus can bind to the same epitope on hNKG2D as 16F16. 16F31, MS, or 21F2. In a preferred embodiment, the antibody that binds to the same epitope on hNKG2D as 16F16, 16F31, MS or 21F2 is a human monoclonal antibody. Such human monoclonal antibodies can be prepared and isolated as described in the Examples.

In another preferred embodiment, the antibody binds to a different epitope than any of the mouse monoclonal antibodies ON72, BAT221, 5C6, 1D11, ECM217, and 149810, and cross-competes more with 16F16, 16F31, MS, or 21F2 than with either of the listed mouse monoclonal antibodies.

In one embodiment, the epitope of an antibody of the invention comprises one or more residues selected from Lys 150, Ser 151, Tyr 152, Thr 180, He 181. Ile 182, Glu 183, Met 184, Gln 185, Leu 191, Lys 197, Tyr 199, Glu 201, Thr 205, Pro 206, Asn 207 and Thr 208 of hNKG2D. In one embodiment, the epitope of an antibody of the invention comprises 5 or more residues selected from Lys 150, Ser 151. Tyr 152, Thr 180, Ile 181, Ile 182, Glu 183, Met 184, Gln 185, Leu 191, Lys 197, Tyr 199, Glu 201, Thr 205, Pro 206, Asn 207 and Thr 208 of hNKG2D. In one embodiment, the epitope of an antibody of the invention comprises 8, 10, 12 or more residues selected from Lys 150, Ser 151, Tyr 152, Thr 180. Ile 181, Ile 182, Glu 183, Met 184, Gln 185, Leu 191, Lys 197, Tyr 199, Glu 201, Thr 205, Pro 206, Asn 207 and Thr 208 of hNKG2D (SEQ ID NO: 9). In one embodiment, the epitope of an antibody of the invention comprises the residues Lys 150, Ser 151, Tyr 152, Thr 180, Ile 181. Ile 182, Glu 183. Met 184, Gln 185, Leu. 191, Lys 197, Tyr 199, Glu 201, Thr 205, Pro 206, Asn 207 and Thr 208 of hNKG2D (SEQ ID NO: 9). In one embodiment, the epitope of an antibody of the invention consists essentially of the residues Lys 150, Ser 151, Tyr 152, Thr 180, Ile 181, Ile 182, Glu 183, Met 184, Gln 185, Lou 191, Lys 197, ‘Tyr 199, Glu 201, Thr 205, Pro 206, Asn 207 and Thr 208 of hNKG2D. In one embodiment, the epitope of an antibody of the invention consists of one or more residues selected from Lys 150, Ser 151, Tyr 152, Thr 180, Ile 181, Ile 182, Glu 183, Met 184, Gln 185, Leu 191. Lys 197, Tyr 199, Glu 201, Thr 205, Pro 206, Asn 207 and Thr 208 of hNKG2D. In one embodiment, the epitope of an antibody of the invention consists of the residues Lys 150, Ser 151, Tyr 152, Thr 180, Ile 181, Ile 182, Glu 183, Met 184, Gln 185, Leu 191, Lys 197, Tyr 199, Glu 201, Thr 205, Pro 206, Asn 207 and Thr 208 of hNKG2D.

In one embodiment, the epitope of an antibody of the invention comprises one or more residues involved in hydrogen-binding selected from Lys 150, Ser 151, Tyr 152, He 181, Met 184, Gln 185, Lys 197, Thr 205, and Asn 207 of hNKG2D (SEQ ID NO: 9). In one embodiment, the epitope of an antibody of the invention comprises 5 or more residues involved in hydrogen-binding selected from Lys 150, Ser 151, Tyr 152, Ile 181, Met 184, Gln 185, Lys 197, Thr 205, and Asn 207 of hNKG2D. In one embodiment, the epitope of an antibody of the invention comprises Lys 150, Ser 151, Tyr 152, Ile 181, Met 184, Gln 185, Lys 197, Thr 205, and Asn 207 of hNKG2D.

Preferred antibodies of the invention exhibit at least one, more preferably two, three, four, five or more, of the following properties: (a) prevents NKG2D-mediated activation of an NKG2D-expressing NK or T cell, optionally with an EC50 for reducing ligand-induced cytotoxicity lower than the EC50 for binding to the cell; (b) competes with at least one NKG2D ligand in binding to NKG2D, preferably with at least MICA and ULBP3; (c) reduces the amount of NKG2D on the surface of a NKG2D-expressing NK or T cell, preferably with at least 75%: (d) binds to cynomolgous and/or rhesus NKG2D, preferably with no less than 50% of the affinity by which it binds to hNKG2D, (e) binds to more than one form or conformation of NKG2D; (f) binds to NKG2D with a Kd of 1 nM or less, preferably 0.1 nM or less; (g) competes with one or more of 16F16, 16F31, MS, or 21F2 in binding to hNKG2D, (h) competes more with 16F16, 16F31, MS, or 21F2 than with any of ON72, BAT221, 5C6, 1D11, ECM217, and 149810 in binding to hNKG2D; (i) blocks more than 90% of 16F16, MS, or 21F2 binding to cell-surface hNKG2D; (j) has insignificant agonist activity, and (k) binds to essentially the same epitope as any of 16F16, 16F31, MS and/or 21F2, preferably essentially the same epitope as MS and/or 21F2. Any combination of the above-described functional features, and/or the functional features as described in the Examples, may be exhibited by an antibody of the invention.

### Structural Properties

Preferred antibodies of the invention are the human monoclonal antibodies 16F16, 16F31, MS, and 21F2 produced, isolated, and structurally and functionally characterized as described in U.S. Pat. No. 7,879,985 incorporated herein by reference.

### Antigen-Binding Fragments

The anti-hNKG2D antibodies of the invention may be prepared as full-length antibodies or antigen-binding fragments thereof. Examples of antigen-binding fragments include Fab, Fab′, F(ab)₂, F(ab′)₂, F(ab)s, Fv (typically the VL and VH domains of a single arm of an antibody), single-chain Fv (scFv; see e.g., Bird et al., Science 1988; 242:423-426; and Huston et al. PNAS 1988; 85:5879-5883), dsFv, Fd (typically the VH and CH1 domain), and dAb (typically a VH domain) fragments; VH, VL, VhH, and V-NAR domains; monovalent molecules comprising a single VH and a single VL chain; rninibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23:1126-1136; WO2005040219, and published. U.S. Patent Applications 20050238646 and 20020161201.

Antibody fragments can be obtained using conventional recombinant or protein engineering techniques, and the fragments can be screened for antigen-binding or other function in the same manner as are intact antibodies.

Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al., Science, 229:81 (1985)). However, these fragments can now be produced directly by recombinant host cells. Alternatively, Fab′-SH fragments can be directly recovered from E. coli and chemically coupled to form F(ab′)2 fragments (Carter et al., Bio/Technology, 10:163-167 (1992)). According to another approach, F(ab′)2 fragments can be isolated directly from recombinant host cell culture. In other embodiments, the antibody of choice is a single-chain fragment (say). See WO 1993/16185; U.S. Pat. No. 5,571,894; and U.S. Pat. No. 5,587,458. The antibody fragment may also be a “linear antibody”, e.g., as described in U.S. Pat. No. 5,641,870, for example. Such linear antibody fragments may be monospecific or bispecific.

### Multispecific Molecules

In another aspect, the present invention features multispecific molecules comprising an anti-hNKG2D antibody, or an antigen-fragment thereof, of the invention. Such multispecific molecules include bispecific molecules comprising at least one first binding specificity for hNKG2D and a second binding specificity for a second target epitope.

One type of bispecific molecules are bispecific antibodies. Bispecific antibodies are antibodies that have binding specificities for at least two different epitopes. Methods for making bispecific antibodies are known in the art, and traditional production of full-length bispecific antibodies is usually based on the coexpression of two immunoglobulin heavy-chain-light-chain pairs, where the two chains have different specificities (Millstein et Nature, 305: 537-539 (1983)). Bispecific antibodies can be prepared as full-length antibodies or antibody fragments (e.g. F(ab′)2 bispecific antibodies) or any other antigen-binding fragments described herein.

In the bispecific antibodies according to the present invention, at least one binding epitope is on the hNKG2D protein. The anti-NKG2D-binding moiety may be combined with second moiety that binds to a molecule on a pro-inflammatory leukocyte, e.g., a T-cell receptor molecule (e.g. CD2, CD3, CD4 or CD8), so as to focus cellular defense mechanisms to a pro-inflammatory hNKG2D-expressing cell. In this embodiment, the bispecific antibodies can, e.g., be used to direct cytotoxic agents to, or an ADCC/CDC attack on, pro-inflammatory cells that express NKG2D. The cytotoxic agent could be, e.g., saporin, an anti-interferon-alpha agent, a vinca alkaloid, the ricin A chain, methotrexate, or a radioactive isotope.

In another embodiment, the second moiety binds a cell-associated target that is presented on or expressed by cells associated with a disease state normally regulated by effector lymphocytes, such as cancer, viral infection, or the like. Thus, for example, a typical target may be a cell stress-associated molecule such as a MIC molecule (e.g., MIC-A or MIC-B) or a ULBP (e.g., Rae-1, H-60, ULBP2, ULBP3, HCMV UL18, or Rae-1β) or a pathogen-associated molecule such as a viral hemagglutinin.

Other multispecific molecules include those produced from the fusion of a hNKG2D-binding antibody moiety to one or more other non-antibody proteins. Such multispecific proteins and how to construct them have been described in the art. See, e.g., Dreier et al. (Bioconjug. Chem. 9(4): 482-489 (1998)); U.S. Pat. No. 6,046,310; U.S. Patent Publication No. 20030103984; European Patent Application 1 413 316; US Patent Publication No. 20040038339, von Strandmann et al., Blood (2006; 107:1955-1962), and WO 2004056873. According to the present invention, the non-antibody protein could be, for example, a suitable ligand for any of the antigens of “second moiety” described I the preceding section; e.g., a ligand for a T-cell or Fc receptor, or a cell-stress molecule such as MIC-A, ULBP, or a pathogen-associated molecule such as a viral hemagglutinin.

Multispecific molecules with more than two valencies are also contemplated. For example, trispecific antibodies can be prepared. Tutt et al J, immunol, 147: 60 (1991). The multispecific molecules of the present invention can be prepared by conjugating the constituent binding specificities using methods known in the art. For example, each binding specificity of the multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB) o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Aced. Sci. USA 82:8648). Other methods include those described in Paulus (1985) Behring Ins, Mitt, No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulthydryl bonding of the C-terminus hinge regions of the two heavy chains. In a particularly preferred embodiment, the hinge region is modified to contain an odd number of sulthydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)2 or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described or reviewed in, for example in U.S. Pat. Nos. 5,260,203; 5,455,030; 4,881,175, 5, 132,405; 5,091,513; 5,476,786; 5,013,653; 5,258,498; 5,482858; U.S. Patent application publication 20030078385, Kontermann et al., (2005) Acta Pharmacological Sinica 26 (1): I-9; Kostelny et al., (1992) J. Immunol, 148 (5):1547-1553; Hollinger et al., (1993) PNAS (USA) 90:6444-6448; and Gruber et al. (1994) J. Immunol, 152: 5368.

### Antibody Variants

An antibody of the invention further can be prepared using an antibody having one or more of the VH and/or VL sequences disclosed herein as starting material to engineer a modified antibody or antibody “variant”, which modified antibody may have altered properties from the parent antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody. Additionally, from antigen-binding portions of an antibody, other constructs such as antigen-binding fragments, antibody derivatives, immunoconjugates, and multispecific molecules can be prepared. Standard molecular biology techniques can be used to prepare and express the altered antibody sequence.

Though an antibody variant or derivative typically has at least one altered property as compared to the “parent” antibody, the antibody variant or derivative can retain one, some or most of the functional properties of the anti-hNKG2D antibodies described herein, which functional properties include, but are not limited to: (a) prevents NKG2D-mediated activation of an NKG2D-expressing NK or T cell, optionally with an EC50 for reducing ligand-induced cytotoxicity lower than the EC50 for binding to the cell; (b) competes with at least one NKG2D ligand in binding to NKG2D preferably with at least MICA and ULBP3; (c) reduces the amount of NKG2D on the surface of a NKG2D-expressing NK or T cell, preferably with at least 75%; (d) binds to cyn.omolgous and/or rhesus NKG2D, preferably with substantially similar efficacy or affinity; (e) binds to more than one form or conformation of NKG2D; (0 binds to NKG2D with a Kd of I nM or less, preferably 0.1 nM or less; (g) competes with one or more of 16F16, 16F31, MS, or 21F2, (h) competes more with 16F16, 16F31, MS, or 2 IF2 than with any of ON72, BAT221, 5C6, I D11, ECM217, and 149810 in binding to hNKG2D; (i) blocks more than 90% of 16F16, MS, or 21F2 binding to cell-surface hNKG2D; (j) has less agonist activity on hNKG2D than any of ON72, BAT221, 5C6, ID11, EGM217, and 149810. Any combination of the above-described functional features, and/or the functional features as described in the Examples, may be exhibited by an antibody of the invention.

The functional properties of the antibody variants and derivatives can be assessed using standard assays available in the art and/or described herein. For example, the ability of the antibody to bind hNKG2D can be determined using standard binding assays, such as those set forth in the Examples (e.g., Biacore, flow cytometry, or ELISAs).

### Nucleic Acids

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule. While the following paragraphs refer to DNA sequences or use thereof, the same methods or principles can generally be applied to mRNA sequences.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acids encoding the antibody can be recovered from the library.

Preferred nucleic acids molecules of the invention are disclosed in U.S. Pat. No. 7,879,985 incorporated herein by reference.

### Single Nucleotide Polymorphisms (SNPs)

In one aspect of the invention, genetic polymorphisms in the genes for the NKG2D receptor and/or NKG2D ligands of subjects were evaluated. In one embodiment, the NKG2D ligand is MICB. TheMICB-rs2239705 SNP is a variant in the MICB gene associated with expression levels of the MICB protein which is a known ligand for the NKG2D receptor (Available from: http://www.ncbi.nlm.nih.gov/SNP/ and the MICB [NKG2D ligand] SNP rs2239705 (Database of Single Nucleotide Polymorphisms (dbSNP). Bethesda (MD): National Center for Biotechnology Information, National Library of Medicine. dbSNP accession:122397051, (dbSNP Build ID: 11501). The rs2255336 SNP is a variant in the NKG2D receptor gene associated with expression levels of NKG2D protein (Available from: http://www.ncbi.nlm.nih.gov/SNP/ and the Database of Single Nucleotide Polymorphisms (dbSNP). Bethesda (MD): National Center for Biotechnology Information, National Library of Medicine. dbSNP accession: {2255336, (dbSNP Build ID: {150}). A post hoc analysis of efficacy data from the NKG2D antibody clinical trial for Crohn's disease demonstrated greater efficacy of the NKG2D antibody in a subgroup of subjects with the rs2255336 SNP.

| Gene Name | Chromosome | Marker | Alleles |
|---|---|---|---|
| KLRK1 | 12:10379727 | rs2255336 | A/G |
| MICB | 6:31545625 | rs2239705 | C/T |

### Antibody Production

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

One preferred animal system for preparing hybridomas is the murine system. Immunization protocols and techniques for isolation of immunized splenocytes for fusion are known in the art, as are fusion partners (e.g., murine myeloma cells) and fusion procedures. Chimeric or humanized antibodies of the present invention can also be prepared based on the sequence of a murine monoclonal antibody using established techniques. For example, DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against hNKG2D can be generated using transgenic or transchromosomic mice carrying parts of the human immune system rather than the mouse system. These transgenic and transchrotnosomic mice include mice referred to herein as HuMAb mice and KM mice, respectively, and are collectively referred to herein as “human Ig mice.” The HuMAb mouse (Medarex, Inc.) contains human immunoglobulin gene miniloci that encode unrearranged human heavy (p and y) and K light chain immunoglobulin sequences, together with targeted mutations that inactivate the endogenous, u and K chain loci (see e.g., Lonberg, et al. (1994) Nature 368: 856-859). Accordingly, the mice exhibit reduced expression of mouse IgM or K, and, in response to immunization, the introduced human heavy and light chain transgenes undergo class switching and somatic mutation to generate high affinity human IgG1(monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y, Acad. Sci. 764:536-546). The preparation and use of Hu Mab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1 993) Nature Genetics 4: 117-123; Chen, J. et al. (1993) EMBO J. 12: 821-830; Tuaillon et al. (1994) J. Immunol. 152:2912 2920; Taylor, L. et al. (1994) International immunology 6: 579-591; and Fishwild, D. et al, (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No, 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al. In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, are described in detail in PCT Publication WO 02/43478 to Ishida et al. Still further, alternative transgenic animal systems expressing human immunoglobulin genes are available in the art and can be used to raise anti-hNKG2D antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al. Moreover, alternative transchromosomic animal systems expressing human immwmglobulin genes are available in the art and can be used to raise anti-hNKG2D antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-hNKG2D antibodies of the invention.

Human monoclonal antibodies of the invention can also be prepared using phage display methods for screening libraries of human irnmunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

When human Ig mice are used to raise human antibodies of the invention, such mice can be immunized with a purified or enriched preparation of hNKG2D antigen and/or cells expressing hNKG2D, as described by Lonberg, N. et al. (1994) Nature 368 (6474): 856-859; Fishwild, D. et al. (1996) Nature Biotechnology, 14: 845-851; and PCT Publication WO 98/24884 and WO 01/14424. Preferably, the mice will be 6-16 weeks of age upon the first infusion. For example, a purified or enriched preparation (5-50 of hNKG2D antigen can be used to immunize the human Ig mice intraperitoneally. In the event that immunizations using a purified or enriched preparation of hNKG2D antigen do not result in antibodies, mice can also be immunized with cells expressing hNKG2D, e.g., a human NK or T-cell line, or a mammalian cell expressing recombinant hNKG2D with or without DAP10, to promote immune responses.

Detailed procedures to generate fully human monoclonal antibodies to hNKG2D are described in Example 1 below. The form and amount of antigen administered (e.g., hNKG2D polypeptide or cell expressing hNKG2D), as well as administration schedules and the possible use of adjuvants such as, e.g., complete Freund's adjuvant or incomplete Freund's adjuvant, are typically optimized for each antigen-mouse system according to established methods in the art.

The immune response can be monitored over the course of the immunization protocol with plasma samples being obtained by retroorbital bleeds, and the plasma or serum can be screened by ELISA (as described below), and mice with sufficient titers of anti-hNKG2D human immunoglobulin can be used for fusions. Mice can be boosted intravenously with antigen 3 days before sacrifice and removal of the spleen. It is expected that 2-3 fusions for each immunization may need to be performed.

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC. CRL 1580) with 50% PEG. Alternatively, the cells can be fused by electrofusion. Cells are plated at approximately 2x10⁵in a flat bottom microliter plate, followed by a two week incubation in selective medium containing 20% fetal Clone Serum, 18% “653” conditioned media, 5% origen (IGEN), 4 mol L-giutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal NM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization. To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Elated IgG can be checked by gel electrophoresis and high performance liquid chromatography to ensure purity. The buffer solution can be exchanged into PBS, and the concentration can be determined by, spectroscopy. The monoclonal antibodies can be aliquoted and stored at −80°.

Antibodies of the invention can also be produced in a host cell transfectoma using, for example, a combination of recombinant DNA techniques and gene transfection methods as is well known in the art (e.g., Morrison, S. (1985) Science 229:1202),

For example, to express the antibodies, DNAs encoding partial or full-length light and heavy chains, can be obtained by standard molecular biology techniques (e.g. PCR amplification or cDNA cloning using a hybridoma that expresses the antibody of interest) and the DNAs can be inserted into expression vectors such that the genes are operatively linked to transcriptional and translational control sequences and may serve their intended function of regulating the transcription and translation of the antibody gene.

The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g. polyadenylation signals)that control the transcription or translation of the antibody chain genes. Such regulatory sequences are described, for example, in Goeddel (Gene Expression Technology. Methods in Enzymology 185, Academic Press, San Diego, Calif. (1990)).

It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or p-globin promoter. Still further, regulatory elements composed of sequences from different sources, such as the SRa promoter system, which contains sequences from the SV40 early promoter and the long terminal repeat of human T cell leukemia virus type 1 (Takebe, Y. et al. (1988) Mol. Cell. Biol. 8:466-4723.

In addition to the antibody chain genes and regulatory sequences, the recombinant expression vectors of the invention may carry additional sequences, such as sequences that regulate replication of the vector in host cells (e.g. origins of replication) and selectable marker genes. The selectable marker gene facilitates selection of host cells into which the vector has been introduced (see, e.g. U.S. Pat. Nos. 4,399,216.4, 634.665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryofic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood. C. R. (1985) immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R J. Kaufman and P. A. Sharp (1982) Biol. 159:601-621), NSO myeloma cells. COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Antibody Characterization

After production or purification, or as part of a screening or selection procedure, the functional characteristics of an anti-hNKG2D antibody of the invention can be investigated. Functional properties of interest include, e.g., antibody binding specificity for hNKG2D, antibody competition with hNKG2D-ligands, antibody competition with reference antibodies (such as, e.g., 16F16, 16F31, MS, and 21F2), the epitope to which the antibody binds, the affinity of the antibody-antigen interaction, and antagonistic/agonistic properties of the antibody.

The following are brief descriptions of exemplary assays for antibody characterization. Some are further described in subsequent sections and/or described in the Examples.
(1) Antibody specificity for hNKG2D can be evaluated by confirming that the monoclonal antibody (or, as part of animal screening procedures, serum containing polyclonal antibodies) binds NKG2D expressing cells but not NKG2D negative cells. Cell lines with or without NKG2D are incubated with antibody followed by incubation with secondary antibody directly labelled, and visualised by, flow cytometry,
(2) Blockade of ligand binding can be evaluated by incubating cells expressing NKG2D with or without antibody or hybridoma supernatant, followed by incubation with a ligand-mFc protein and a secondary antibody specific for the ligand, and the level of ligand binding and blockade thereof determined by flow cytometry. Blockade can be calculated as the % ligand binding with pre-incubation compared to without pre-incubation, when lower binding is seen upon pre-incubation.
(3) Competition for binding site used by one or more reference anti-NKG2D antibodies can be evaluated in a similar manner, except that the pre-incubation can either performed with an antibody of the invention or the reference antibody (e.g., ON72 or 149810), followed by incubation with and detection of the subsequently added antibody.
(4) Affinity parameters, including on- and off-rate, of antibodies can determined on a Biacore machine. For example, hNKG2D-Fc protein can be immobilized on a chip, the antibody passed over the chip, the on- and off-rates determined, and the KD calculated.
(5) Induction of NKG2D internalisation by antibodies can be measured by incubating hNKG2D-expressing cells with or without antibody overnight, followed by re-addition of the antibody and detection of the level of NKG2D (i.e. the level of antibody bound) in a flow cytometer.
(6) The ability of an antibody to block hNKG2D-ligand mediated killing can be assessed, using, e.g., the NK cell lines NK92 or NKL as effector cells that kill ⁵¹Cr-loaded target cells expressing NKG2D ligand, either MICA, MICB, or ULBP1.-4.
(7) Cross-reactivity of the human anti-NKG2D antibodies with monkey NK and CD8+ T cells but not CD4+ T cells (as in humans), can be demonstrated by flow cytometry after incubation of monkey and human PBMC's with hNKG2D antibody and secondary antibody, along with markers of the different cell types in PBMCs, and analysing NKG2D staining of the various subsets.
(8) Activation of NKG2D upon antibody binding can be measured as induction of cell-proliferation of CD8+ cells in a PBMC population upon stimulation via the T-cell receptor, CD28 and or NKG2D, with or without pre-stimulation (e.g., via TCR, CD28 and IL-2 or IL-15).

### Binding Assays

The present invention provides for antibodies, and antigen-binding fragments and immunoconjugates thereof, that bind hNKG2D. Any of a wide variety of assays can be used to assess binding of an antibody to hNKG2D. Protocols based upon ELISAs, radioimmunoassays, Western blotting, BIACORE, and other competition assays, inter alia, are suitable for use and are well known in the art. Further, several binding assays, including competition assays, are described in the Examples in U.S. Pat. No. 7,879,985.

For example, simple binding assays can be used, in which a test antibody is incubated in the presence of a target protein or epitope NKG2D or a portion thereof), unbound antibodies are washed off, and the presence of bound antibodies is assessed using, e.g., radio labels, physical methods such as mass spectrometry, or direct or indirect fluorescent labels detected using, e.g., cytofluorometric analysis (e.g. FACScan). Such methods are well known to those of skill in the art. Any amount of binding above the amount seen with a control, non-specific antibody indicates that the antibody binds specifically to the target. In such assays, the ability of the test antibody to bind to the target cell or human NKG2D can be compared with the ability of a (negative) control protein, e.g. an antibody raised against a structurally unrelated antigen, or a non-Ig peptide or protein, to bind to the same target. Antibodies or fragments that bind to the target cells or NKG2D using any suitable assay with 25%, 50%, 100%, 200%, 1000%, or higher increased affinity relative to the control protein, are said to “specifically bind to” or “specifically interact with” the target, and are preferred for use in the therapeutic methods described below. The ability of a test antibody to affect the binding of a (positive) control antibody against NKG2D. e.g. 16F16, 16F31, MS, or 21F2, may also be assessed.

In one aspect, the invention provides for anti-hNKG2D antibodies sharing biological characteristics and/or substantial VH and/or VL sequence identity with 16F16, 16F31, MS, or 21F2. One exemplary biological characteristic is the binding to the 16F16, 16F31, MS, or 21F2 epitope, i.e., the respective regions in the extracellular domain of hNKG2D to which the 16F16, 16F31, MS, or 21F2 antibodies bind. To screen for antibodies that bind to the 16F16, 16F31, MS, or 21F2 epitope, a routine cross-blocking assay, such as that described in Antibodies, A Laboratory Manual, Cold Spring Harbor Laboratory, Ed Harlow and David Lane (1988), can be performed.

In an exemplary cross-blocking or competition assay. 16F16, 16F31, MS, or 21F2 (control) antibody and a test antibody are admixed (or pre-adsorbed) and applied to a sample containing NKG2D. In certain embodiments, one would pre-mix the control antibodies with varying amounts of the test antibody (e.g., 1:10 or 1:100) for a period of time prior to applying to the NKG2D-containing sample. In other embodiments, the control and varying amounts of test antibody can simply be admixed during exposure to the antigen/target sample. As long as one can distinguish bound from free antibodies (e.g., by using separation or washing techniques to eliminate unbound antibodies) and the control antibody from test antibody (e.g., by using species—or isotype-specific secondary antibodies, by specifically labeling the control antibody with a detectable label, or by using physical methods such as mass spectrometry to distinguish between different compounds) one will be able to determine if the test antibody reduces the binding of the control antibody to the antigen, indicating that the test antibody recognizes substantially the same epitope as the control. In this assay, the binding of the (labeled) control antibody in the presence of a completely irrelevant antibody is the control high value. The control low value is be obtained by incubating the labeled (positive) control antibody with unlabeled control antibody, where competition would occur and reduce binding of the labeled antibody.

In a test assay, a significant reduction in labeled antibody reactivity in the presence of a test antibody is indicative of a test antibody that recognizes the same epitope, i.e., one that “cross-reacts” with the labeled control antibody. Any test antibody or compound that reduces the binding of the labeled control to the antigen/target by at least 50% or more preferably 70%, at any ratio of control:test antibody or compound between about 1:10 and about 1:100 is considered to be an antibody or compound that binds to substantially the same epitope or determinant as the control. Preferably, such test antibody or compound will reduce the binding of the control to the antigen/target by at least 90%. Nevertheless, any compound or antibody that reduces the binding of a control antibody or compound to any measurable extent can be used in the present invention.

In one embodiment, competition can be assessed by a flow cytometry test. Cells bearing hNKG2D are incubated first with a control antibody that is known to specifically bind to the receptor (e.g., T or NK cells expressing hNKG2D or BaF/3 cell recombinantly expressing hNKG2D, and 16F16, 16F31, MS, or 21F2 antibody), and then with the test antibody that may be labeled with, e.g., a fluorochrome or biotin. The test antibody is said to compete with the control if the binding obtained with preincubation with saturating amounts of control antibody is 80%, preferably, 50%, 40% or less of the binding (mean of fluorescence) obtained by the antibody without preincubation with the control. Alternatively, a test antibody is said to compete with the control if the binding obtained with a labeled control (by a fluorochrome or biotin) on cells preincubated with saturating amount of antibody to test is 80%, preferably 50%, 40%. or less of the binding obtained without prein.cubation with the antibody. See Example 5 for an exemplary antibody competition assay.

Similar cross-blocking assays can also be used to evaluate whether a test (humanized) antibody affects the binding of a natural ligand for human NKG2D, such as MICA, MICB, ULBP1, ULBP3, ULBP4, or a member of the RAET1 family, simply by exchanging 16F16, 16F31, MS, or 21F2 for a suitable form of the hNKG2D.-ligand. One suitable form, described in the Examples, are fusion proteins of the ligand (e.g., MICA) with the Fc-portion of an antibody. Having the ligand conjugated to an ft-region allows for detection of the fusion protein by antibodies specific for the animal species from which the Fe-region derives, using, e.g., goat-anti-mouse antibodies to detect a murine Fc-region.

In one embodiment, a cellular assay is used in which hNKG2D-expressing cells, e.g., CD4⁺CD28 ⁻ cells from rheumatoid arthritis patients (or the equivalent cells from another autoimmune or inflammatory disorder) are incubated with an NKG2D ligand such as MICA, MICB, or a ULBP protein, e.g., in the form of an Fc-fusion protein, or a cell expressing any of these ligands, and the ability of an anti-NKG2D antibody or other molecule to block the activation of the cell is assessed. In an alternative assay, a baseline level of activity for the NKG2D receptor is obtained in the absence of a ligand, and the ability of the antibody or compound to cause a decrease in the baseline activity level is detected. In one type of embodiment, a high-throughput screening approach is used to identify compounds capable of blocking the activation of the receptor, or otherwise downregulating it. See Example 3 for an exemplary ligand competition assay.

Preferably, monoclonal antibodies that recognize an NKG2D epitope will react with an epitope that is present on a substantial percentage of CD4+ T cells, particularly CD4+CD28− T cells, in patients such as rheumatoid arthritis patients, but will not significantly react with other cells, i.e., immune or non-immune cells that do not express NKG2D. Accordingly, once an antibody that specifically recognizes hNKG2D on NK or T cells, it can be tested for its ability to bind to T cells taken from patients with autoimmune or inflammatory disorders such as rheumatoid arthritis. It will be appreciated that the present invention can be used for the treatment of any disorder in which NKG2D activity is linked to the pathology of the disorder, regardless of the cell type expressing the receptor (e.g., CD4+ T cells, CD8+ T cells, NK cells, etc.), and the antibodies can be tested for their ability to bind to the receptor on whichever cell type is relevant for the particular disorder. For example, if it is observed that a particular disorder is associated with excess activity or proliferation of NKG2D-expressing NK cells, then the antibodies can be developed and tested using NK cells expressing the same receptor.

In one embodiment, the antibodies are validated in an immunoassay to test its ability to bind to NKG2D-expressing cells, e.g. CD4+CD28− T cells taken from patients with rheumatoid arthritis. For example, peripheral blood lymphocytes (PBLs) are taken from a plurality of patients, and CD4+, preferably CD4+CD28−, cells are enriched from the PBLs, e.g., by flow cytometry using relevant antibodies. The ability of a given antibody to bind to the cells is then assessed using standard methods well known to those in the art. Antibodies that are found to bind to a substantial proportion (e.g., 20%, 30%, 40%, 50%, 60%, 70%, 80% or more) of cells known to express NKG2D, e.g. NK cells, CD8 T cells, CD4 T cells from RA patients, etc., from a significant percentage of patients (e.g., 5%, 10%, 20%, 30%, 40%, 50% or more) can be deemed suitable for use in the present invention, both for diagnostic purposes to determine the expression of the NKG2D receptor in a patient's cells or for use in the herein-described therapeutic methods, e.g., for use as human-suitable blocking or, alternatively, cytotoxic antibodies. To assess the binding of the antibodies to the cells, the antibodies can either be directly or indirectly labeled, When indirectly labeled, a secondary, labeled antibody is typically added. The bindituz of the antibodies to the cells can then be detected using, e.g., cytofluorornetric analysis (e.g. FACS). Such methods are well known in the art.

In some aspects of the invention, e.g., where it is not desired to kill NKG2D-expressing cells, the antibodies of the invention preferably do not demonstrate substantial specific binding to Fc receptors. Such antibodies may comprise constant regions of various heavy chains that are known not to bind Fc receptors. One such example is an IgG4 constant region. Alternatively, antibody fragments that do not comprise constant regions, such as Fab or F(ab′)2 fragments, can be used to avoid Fc receptor binding. Fc receptor binding can be assessed according to methods known in the art, including for example testing binding of an antibody to Fc receptor protein in a BIACORE assay. Also, any other antibody type can be used in which the Fc portion is modified to minimize or eliminate binding to Fc receptors (see, e.g., WO03101485, the disclosure of which is herein incorporated by reference). Assays such as, e.g., cell based assays, to assess Fe receptor binding are well known in the art, and are described in, e.g., WO03101485.

### Functional Assays

Any suitable physiological change that reflects NKG2D activity can be used to assess the utility of a test compound or antibody. For example, one can measure a variety of effects in, e.g., cell-based assays, such as changes in gene expression, cytokine production, signalling molecule phosphorylation, cell growth, cell proliferation, pH, intracellular second messengers, e.g., Ca2+, IP3, cGMP, or cAMP, or activity such as cytotoxic activity or ability to activate other T cells. For example, the activity of the receptor can be assessed by detecting the expression of NKG2D-responsive genes, e.g., CD25, IFN-gamma, or TNF-alpha (see, e.g., Groh et al, (2003) PNAS 100: 9452-9457; Andr et al. (2004) Eur. J. Immuno). 34: 1-11). Alternatively, NKG2D activity can be assessed by incubating CD4+CD28−NKG2D+ cells in the presence of a ligand or activating anti-NKG2D antibody, as well as an anti-CD3 antibody, to evaluate the ability of the compound or test antibody to inhibit the release of INF-alpha or IFN-gamma by the cells. Alternatively, CD4+CD28−NKG2D+ T cells can be incubated in the presence of ligand, e.g., MICA, MICB, ULBP-1, ULBP-2, ULBP-3, etc., or ligand-producing cells, e.g., autologous MIC+RA synoviocytes, and the ability of the test antibody or compound to inhibit cytokine production (e.g., IFN-gamma or INF-alpha), or T cell proliferation assessed.

In vitro assays can optionally use cells taken from patients with autoimmune or inflammatory disorders such as RA, e.g. CD4+CD28− cells expressing NKG2D taken from (or cell lines derived therefrom) patients with RA, but in general any NKG2D-expressing cells can be used. For example, non-RA immune cell lines, e.g. T cell lines, can be transfected with an NKG2D-encoding transgene and used in the present assays, so long that the expression of the receptor alters the activity of the cells in a detectable way, e.g., renders them activatible by NKG2D ligand. Cell lines can, for example, be established using CD4+CD28−NKG2D+ cells from RA patients, e.g. PBLs or T cells isolated from synovial tissue. Such cells can be cultured in the presence of 1L-15 to ensure continued expression of NKG2D (see, e.g., Groh et al. (2003) PNAS 100: 9452-9457, the entire disclosure of which is herein incorporated by reference).

If an anti-hNKG2D antibody reduces or blocks NKG2D interactions with one or more of its ligands, or competes with an antibody known to block hNKG2D ligand interaction, it can be useful for reducing NKG2D-mediated activation of NK or T cells. This can be evaluated by a typical cytotoxicity assays. Example 6 describes an exemplary cytotoxicity assay, NKG2D-ligand mediated killing of target cells. Here, the ability of anti-hNKG2D antibodies to reduce or inhibit the NK cell-mediated killing of MICA-transfected BaF/3 is assessed by measuring target cell release of 51Cr.

In other aspects, it may desirable to ensure that antibodies of the invention lack substantial agonistic activity, Several assays can be used for this purpose, including the following.

One assay can evaluate proliferation and cytokine production after activation with antibodies, either soluble or plate-bound, in combination with anti-CD3 and/or anti-CD28 antibodies, of PBMCs from healthy volunteers or IBD patients. In this method, PBMCs are purified by conventional methods from healthy subjects or inflammatory bowel disease (IBD) patients. The cells are stained with CFSE (from Molecular probes, cat #C34554). To 10⁷ cells (in 0.5 ml PBS with 2% FCS) is added 1 μl CFSE (0.5 inM) and the cells are incubated at 37° C. for 10 min. Then, 2 ml FCS is added, and the mixture is left for 1 min at room temperature. The cells are then washed 3 times by centrifugation with RPMI-1640 medium (12 ml). After wash, the cells are resuspended in 1 ml media (e.g. RPMI-1640) with 2% FCS.

Ninety-six well plates are coated with 30 μl anti-mouse Fc (Jackson-Immuno Research 115-006-008) for 2 hours at room temperature, and then washed with PBS. Antibodies (anti-CD3 Biosceince cat:414-0037-82, anti-CD28 cat#348046 Becton Dickison) are added according to the scheme below and left in the well:
Cells Alone
CD3 0.1 or 0.3 ng/ml
CD3 0.1 or 0.3 ng/ml +CD28 0.2 μg/ml
CD3 0.1 or 0.3 ng/ml+CD28 0.2 μg/ml+anti-NKG2D 0.2 μ/ml
CD3 0.1 or 0.3 ng/ml+anti-NKG2D 0.2 μg/ml

Next, 100.000 CFSE-labelled PBMCs are added and left for 3 days. Supernatant is then collected for analysis of cytokines, and the PBMCs are analysed by flow cytometry with regard to the type of lymphocyte with anti-CD56, anti-CD4 anti-CD8, and CFSE labelling for proliferation.

In another assay, the effect on the cytotoxic potential of CD8+ T cells towards a target cells lacking NKG2D ligands, is tested. If binding boosts the cytotoxic potential of the cells, agonistic activity is present. Briefly, IL-2 stimulated PBMC from healthy subjects are incubated with p815 cells expressing MICA, or with untransfected p815 cells and an anti-CD3 antibody, (which will lead to redirected killing by binding to the Fc receptors on p815 cells) and CD8 cytotoxic T cells. It is then analyzed whether an anti-NKG2D antibody that does not bind to p815 cells (e.g., an antibody of human IgG4 isotype) blocks MICA-NKG2D-directed binding and/or if the antibody boosts CD3-p815 redirected binding. In this manner, it can be shown that the activity of the CD8+ T cells is not enhanced by incubating p815 cells with an anti-CD3 antibody and an additional anti-NKG2D antibody, while the same anti-NKG2D antibody can shown to be functional by demonstrating that it blocks NK-MICA interaction on p815-induced killing in the same PBMC population.

In another assay, it can be explored whether NKG2D-signalling pathways and −molecules are activated by addition of one or more anti-NKG2D antibodies. NK cell lines (such as. e.g., NKL cells or NK-92 cells), or human NK or CD8+ T cells isolated from peripheral blood, can be used. For example, NKL cells can be incubated with a human anti-NKG2D antibody in solution or plate bound, with, e.g., Fe-MICA or irradiated MICA expressing cells as a control. After incubation for suitable time periods, (e.g., 5, 10, 30 min), the cells are lysed in the presence of protease and phosphatase inhibitors on ice, and analyzed for the levels of one or more phosphorylated signalling molecules that are known to he downstream of stimulation of NKG2D (e.g., Pi3K, Akt, and vav), by standard Western blotting techniques.

In animal-based assays, any physiological or pathological consequence of NKG2D activation in cells within the animal can be used to assess antibody or test compound activity. For example, CD4+CD28−NKG2D+ cells can be introduced into the joints of an animal model, with or without co-administration of ligand producing cells such as MICA-producing synoviocytes, and inflammation or tissue damage is assessed. Test compounds or antibodies can then be introduced, and their ability to inhibit, slow, reverse, or in any way affect the inflammation or tissue damage is detected.

Experiments with rheumatoid arthritis (RA) synovial explants can also be performed to study the effects of blocking NKG2D on spontaneous release of pro-inflammatory cytokines (see, e,g., Brennan et al., Lancet 1989; 2 (8657); 244-247). In such an assay, human or humanized anti-hNKG2D antibodies are tested on RA synovial membrane cultures and compared to, e.g., murine anti-hNKG2D antibodies at concentrations shown to be useful to block ligand binding and function of NKG2D. RA synovial cells are cultured for 48 hrs in the absence or presence of anti-NKG2D antibodies or an isotype control antibody. Known anti-inflammatory drugs can be used as positive controls. The effects of the anti-NKG2D antibodies are initially tested at concentrations up to 30 μg/ml on 6 RA synovial membranes. Viability of the cells is analysed in a assay staining living cells (e.g. a MTT assay) to determine if the added reagent has any cytotoxicity. ELISA is then used to detect cytokines such as, e.g., TNF-α, IL-1β and IL-6 levels in culture supernatants.

Alternatively, antibodies of the invention can be tested in experimental models of, e.g., psoriasis or ulcerative colitis. Psoriasis-affected skin sample can be transplanted onto a SCID mouse together with the patients own PBMC's, and the effect of introduction of a test compound and their ability to inhibit, slow, reverse, or in any way affect the inflammation or tissue damage, can be detected. Kjellev et al. (Eur J Immunol 2008; 37:1397-1406) and Ito et al. (Am J Physiol Gastrointest Liver Phvsiol 2008; 294:G199-G207) describe experimental models for assessing treatment of ulcerative colitis using anti-murine NKG2D antibody.

### Pharmaceutical Formulations

In one embodiment, the present invention provides a pharmaceutical composition or formulation comprising anti-hNKG2D antibodies as described herein together with one or more carriers.

Accordingly, one exemplary aspect of the invention is a pharmaceutical formulation comprising such an antibody which is present in a concentration from 1 mg/ml to 500 mg/ml, and wherein said formulation has a pH from 2.0 to 10.0. The formulation may further comprise a buffer system, preservative(s), tonicity agent(s), chelating agent(s), stabilizers, and/or surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient may add solvents and/or diluents prior to administration. In another embodiment, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/mi or above, and wherein said formulation has a pH from about 2.0 to about 10.0.

In a another embodiment, the pH of the formulation is in the range selected from the list consisting of from about 2.0 to about 10.0, about 3.0 to about 9.0, about 4.0 to about 8,5, about 5.0 to about 8.0, and about 5.5 to about 7.5.

In a further embodiment, the formulation includes a buffer that is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation also or alternatively comprises a pharmaceutically acceptable preservative. The preservative may be selected from, e.g., the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol and thiomerosal, bronopol. benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. The preservative may, e.g., be present in a concentration from 0.1 mg/ml to 20 mg/ml, from 0.1 mg/ml to 5 mg/ml, from 5 mg/ml to 10 mg/ml, or from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19th edition, 1995.

In a further embodiment, the formulation also or alternatively comprises an isotonic agent. The isotonic agent may be, e.g., selected from the group consisting of a salt (e.g. sodium chloride), a sugar or sugar alcohol, an amino acid (e.g. L-glycine, L histidine, arginine, lyrsirre, isoleucine, aspartic acid, tryptophan, threonine), an alditol (e.g. glycerol (glycerine), 1,2-propanediol (propyleneglycol), 1,3-propanediol, 1,3-butanediol)polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment, the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment, the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. The sugar or sugar alcohol concentration can, e.g., be between about 1 mg/ml and about 150 mg/ml. The isotonic agent can be present in a concentration from, e.g., 1 mg/ml to 50 mg/ml, from 1 mg/ml to 7 mg/ml, from 8 mg/ml to 24 mg/ml, or from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19th edition, 1995.

In a further embodiment, the formulation also or alternatively comprises a chelating agent. The chelating agent can, for example, be selected from salts of ethylenediaminetetraacetic acid (EDTA), citric acid, and aspartic acid, and mixtures thereof. The chelating agent may, for example, be present in a concentration from 0.1 mg/ml to 5 mg/ml, from 0.1 mg/ml to 2 mg/ml, or from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19th edition, 1995.

In a further embodiment of the invention the formulation also or alternatively comprises a stabilizer, The use of a stabilizer in pharmaceutical compositions is well-known to the skilled person. For convenience reference is made to Reminizton: The Science and Practice of Pharmacy, 19th edition, 1995. More particularly, compositions of the invention can be stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci, Technol. 38:48-59), spray drying (see Masters (1991) in Spray-Drying Handbook (5th ed; Longman Scientific and Technical, Essez, U.K.), pp. 491-676; Broadhead et at (1992) Drug Devel. Ind. Pharm. 18:1169-1206; and Mumenthater et al. (1 994) Pharm. Res, 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may alternatively or further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer L, D, or a mixture thereof) of a particular amino add (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, arninoguanidine, ornithine and N-monoethyl L-arginine, suitable methionine analogues include ethionine and buthionine and suitable cysteine analogues include S-methyl-L cysteine, As with the other amino acids, the amino add analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. The term “inhibit” in this context is intended to mean minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment, the formulation further or alternatively comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL and HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also or alternatively comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment, the formulation further or alternatively comprises a surfactant. The surfactant may, for example, be selected from a detergent, ethoxylated castor oil, polyglycolyzed glycerides, acetylated monoglycerides, sorbitan fatty acid esters, poiyoxypropylene-polyoxyethylene block polymers (eg. poloxamers such as Pluronick F68, poloxamer 188 and 407, Triton X-I00), polyoxyethylene sorbitan fatty acid esters, polyoxyethylene and polyethylene derivatives such as alkylated and alkoxylated derivatives (tweens, e.g. Tween-20, Tween-40. Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine choline, serine or threonine) and alkyl, alkoxyl(alkyl ester), alkoxy(alkyl ether)-derivatives of lysophosphatidyl and phosphatidylcholines, e.g. lauro and myristoyl derivatives of lysophosphatidylcholine, dipalmitoylphosphatidylcholine, and modifications of the polar head group, that is cholines, ethanolamines, phosphatidic acid. serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (e.g., oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids. DSS (docusate sodium_{;} CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine: conjugates, ursodeoxycholic acid, sodium cholate, sodium deoxycholate, sodium taurocholate, sodium glycocholate, N-Hexadecyl-N,N-dimethyl-3-ammonio-l-propanesulfonate, anionic (alkyl-aryl-stilphonates) monovalent surfactants, zwitterionic surfactants (e.g. N-alkyl-N,N-ditnethylammonio-l-propariesulfonates, 3-cholamido-1-propyldimethylaminonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetralunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science arid Practice of Pharmacy, 19th edition, 1995.

In a further embodiment, the formulation further or alternatively comprises protease inhibitors such as EDTA (ethylenediamine tetraacetic acid)) and benzamidine HCl, but other commercially available protease inhibitors may also be used. The use of a protease inhibitor is particular useful in pharmaceutical compositions comprising zymogens of proteases in order to inhibit autocatalysis.

It is possible that other ingredients may also or alternatively be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing an antibody according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, tnicroemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the antibody, increase bioavailability, increase solubility, decrease adverse effects, achieve chromotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly (vinyl alcohol), acrylate and methacrylate polymers, polylactic and polyglycolic acid and block copolymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of an antibody, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the antibody compound in the form of a nasal or pulmonal spray. As a still further option, the pharmaceutical compositions containing an antibody of the invention can also be adapted to transdermal administration, e.g. by needle-free injection or from a patch, optionally an iontophoretic patch, or transmucosal, e.g. buccal, administration.

The antibody can be administered via the pulmonary route in a vehicle, as a solution, suspension or thy powder using any of known types of devices suitable for pulmonary drug delivery. Examples of these comprise of, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit. Rev Ther Drug Carr Sys 14 (4) (1997) 395-453).

Based on standardised testing methodology, the aerodynamic diameter (dₐ) of a particle is defined as the geometric equivalent diameter of a reference standard spherical particle of unit density (1 g/cm³). In the simplest case, for spherical panicles, da is related to a reference diameter (d) as a function of the square root of the density ratio as described by:

d a=ρ ρ a d

Modifications to this relationship occur for non-spherical particles (cf. Edwards D A, Ben-Jebria A, Langer R. Recent advances in pulmonary drug delivery using large, porous inhaled particles. J Appl Physiol 84 (2) (1998) 379-385). The terms “MMAD” and MMEAD″ are well-described and known to the art (cf. Edwards D A, Ben-Jebria A, Langer R and represents a measure of the median value of an aerodynamic particle size distribution. Recent advances in pulmonary drug delivery using large, porous inhaled particles. J Appl Physiol 84 (2) (1998) 379-385). Mass median aerodynamic diameter (MMAD) and mass median effective aerodynamic diameter (MMEAD) are used inter-changeably, are statistical parameters, and empirically describe the size of aerosol panicles in relation to their potential to deposit in the lungs, independent of actual shape, size, or density (cf. Edwards D A, Ben-Jebria A. Langer R. Recent advances in pulmonary drug delivery using large, porous inhaled particles. J Appl Physiol 84 (2) (1998) 379-385). MMAD is normally calculated from the measurement made with impactors, an instrument that measures the particle inertial behaviour in air.

In a further embodiment, the formulation could be aerosolized by any known aerosolisation technology, such as nebulisation, to achieve a MMAD of aerosol particles less than 10 μm, more preferably between 1-5 μm, and most preferably between 1-3 μm. The preferred particle size is based on the most effective size for delivery of drug to the deep Bing, where protein is optimally absorbed (cf. Edwards D A, Ben-Jebria. A, Langer A. Recent advances in pulmonary drug delivery using large, porous inhaled particles. J Appl Physiol 84 (2) (1998) 379-385).

Deep lung deposition of the pulmonal formulations comprising the antibody may optional be further optimized by using modifications of the inhalation techniques, for example, but not limited to: slow inhalation flow (eg. 30 L/min), breath holding and timing of actuation.

The term “stabilized formulation” refers to a formulation with increased physical stability, increased chemical stability or increased physical and chemical stability. The term “physical stability” of the protein formulation as used herein refers to the tendency of the antibody to form biologically inactive and/or insoluble aggregates as a result of exposure of the antibody to thermo-mechanical stresses and/or interaction with interfaces and surfaces that are destabilizing, such as hydrophobic surfaces and interfaces. Physical stability of the aqueous antibody formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to antibody aggregation, when it shows visual turbidity in daylight. Alternatively, the turbidity of the formulation can be evaluated by simple turbidity measurements well-known to the skilled person. Physical stability of the aqueous antibody formulations can also be evaluated by using a spectroscopic agent or probe of the conformational status of the antibody. The probe is preferably a small molecule that preferentially binds to a non-native conformer of the antibody. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the antibody formulation as used herein refers to chemical covalent changes in the antibody structure leading to formation of chemical degradation products with potential less biological potency and/or potential increased immunogenic properties compared to the native antibody structure. Various chemical degradation products can be formed depending on the type and nature of the native antibody and the environment to which the antibody is exposed. Elimination of chemical degradation can most probably not be completely avoided and increasing amounts of chemical degradation products is often seen during storage and use of the antibody formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahem, T. I & Manning M. C., Plenum Press, New York 1992). Oxidation (of for instance methionine residues) can be mentioned as another variant of chemical degradation. The chemical stability of the antibody formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the antibody is stable for more than 6 weeks of usage and for more than 3 years of storage.

In another embodiment of the invention the pharmaceutical formulation comprising the antibody is stable for more than 4 weeks of usage and for more than 3 years of storage.

In a further embodiment of the invention the pharmaceutical formulation comprising the antibody is stable for more than 4 weeks of usage and for more than two years of storage.

In an even further embodiment of the invention the pharmaceutical formulation comprising the antibody is stable for more than 2 weeks of usage and for more than two years of storage.

Suitable antibody formulations can also be determined by examining experiences with other already developed therapeutic monoclonal antibodies. Several monoclonal antibodies have been shown to be efficient in clinical situations, such as Rituxan (Rituximab), Herceptin (Trastuzumab) Xolair (Omalizumab), Bexxar (Tositurnomab), Campath (Alemtuzumab), Zevalin, Oncolym, Humira and similar formulations may be used with the antibodies of this invention. For example, a monoclonal antibody can be supplied at a concentration of 10 mg/mL in either 100 mg (10 mL) or 500 mg (50 mL) single-use vials, formulated for IV administration in 9.0 mg/mL sodium chloride, 7.35 mg/mL sodium citrate dihydrate, 0.7 mg/mL polysorbate 80, and sterile water for injection. The pH is adjusted. to 6.5. Alternatively, the antibody can be formulated in a solution comprising histidin, sucrose, and Polysorbate 80.

### Diagnostic Applications

The hNKG2D-antibodies of the invention also have non-therapeutic applications. For example, anti-hNKG2D antibodies may also be useful in diagnostic assays for NKG2D protein, e.g. detecting its expression in specific cells, tissues, or serum. For example, anti-hNKG2D antibodies could be used in assays selecting patients for anti-hNKG2D treatment. For such purposes, the anti-hNKG2D antibodies could be used for analyzing for the presence of hNKG2D in serum or tissue specimens, testing for the presence of CD4+ T cells expressing NKG2D, or the presence of disease promoting cells expressing NKG2D (e.g., NK or CD4 + or CD8+ T cells). Such analyses could be combined with analyses testing, e.g., for the levels of soluble MICA in blood (see, e.g., WO2003089616 by Spies et al.).

For diagnostic applications, the antibody typically will be labeled with a detectable moiety. Numerous labels are available that can be generally grouped into the following categories:
(a) Radioisotopes, such as ³⁵S, ⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.
(b) Fluorescent labels such as rare-earth chelates (europium chelates) or fluorescein and its derivatives, rhodamine and its derivatives, dansyl, Lissamine, phycoeiythrin and Texas Red are available. The fluorescent labels can be conjugated to the antibody using the techniques disclosed in Current Protocols in Immunology, supra, for example, fluorescence can be quantified using a fluorimeter.
(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase U.S. Pat. No. 4,737,456). luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharde oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al, “Methods for the Preparation of Enzyme-Antibody Conjugates for use in Enzyme Immunoassay,” in Methods in Enzym. (Ed., J. Langone & H. Van Vunakis)Academic Press, New York, 73:147-166 (1981). Examples of enzyme-substrate combinations include, for example:
(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-nitrophenyl phosphate as chromogenic substrate; and
(iii) beta-D-galactosidase (beta-D-Gal) with a chromogenic substrate (e.g., p-nitrophenyl-beta-D-galactosidase) or fluorogenic substrate 4-methylumbelliferyl-p-beta-galactosidase. Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin, and any of the three broad categories of labels mentioned above can be conjugated with avidin, or vice versa. Biotin binds selectively to avidin, and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment of the invention, the anti-N KG2D antibody need not be labeled, and the presence thereof can be detected using a labeled secondary antibody that binds to the NKG2D antibody.

The antibodies of the present invention may be employed in any known assay method, such as competitive-binding assays, direct and indirect sandwich assays, and immunoprecipitation assays. Zola, Monoclonal Antibodies: A Manual of Techniques, pp. 147-158 (CRC Press, Inc. 1987).

For immunohistochemistry, the tissue sample may be fresh or frozen or may be embedded in paraffin and fixed with a preservative such as formalin, for example. The antibodies may also be used for in vivo diagnostic assays. Generally, the antibody is labeled with a radionuclide or a non-radioactive indicator detectable by, e.g., nuclear magnetic resonance, or other means known in the art. Preferably, the label is a radiolabel, such as, e.g., ¹²⁵I, ¹³¹I, 67Cu, ^{99m}Tc, or ¹¹¹In. The labeled antibody is administered to a host, preferably via the bloodstream, and the presence and location of the labeled antibody in the host is assayed. This imaging technique is suitably used in the detection, staging and treatment of neoplasms. The radioisotope is conjugated to the protein by any means, including metal-chelating compounds or lactoperoxidase, or iodogen techniques for iodination.

As a matter of convenience, the antibodies of the present invention can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor that provides the detectable chromophore or fluorophore). In addition, other additives may he included such as stabilizers, buffers (e.g., a block buffer or lysis buffer) and the like. The relative amounts of the various reagents may be varied widely to provide for concentrations in solution of the reagents that substantially optimize the sensitivity of the assay. Particularly, the reagents may be provided as dry powders, usually lyophilized, including excipients that on dissolution will provide a reagent solution having the appropriate concentration.

### Therapeutic Applications

Methods of treating a patient using a human or humanized anti-hNKG2D antibody as described herein are also provided for by the present invention. In one embodiment, the invention provides for the use of a human or humanized antibody as described herein in the preparation of a pharmaceutical composition for administration to a human patient. Typically, the patient suffers from, or is at risk for, an autoimmune or inflammatory disease or disorder. For example, in one aspect, the invention provides a method of reducing or inhibiting hNKG2D-mediated activation of NK or T cells in a patient in need thereof, comprising the step of administering a human or humanized anti-NKG2D antibody to the patient, which antibody reduces or prevents ligand-mediated activation of the NKG2D receptor. In one embodiment, the method directed at decreasing the activity of such lymphocytes in patients having a disease in which increased NK or T cell activity is detrimental which involves, affects or is caused by cells susceptible to lysis by NK or T cells, or which is caused or characterized by increased NK and/or T cell activity, such as an autoimmune disease or disorder or an inflammatory condition in one aspect, the invention provides a method of reducing chronic inflammation in the patient.

Exemplary conditions or disorders to be treated with the polypeptides, antibodies and other compounds of the invention, include, but are not limited to systemic lupus erythematosis, rheumatoid arthritis, juvenile chronic arthritis, psoriatic arthritis, osteoarthritis, spondyloarthropathies (ankylosing spondylitis), systemic sclerosis (scleroderma), idiopathic inflammatory myopathies (dermatomyositis, polymyositis), Sjogren's syndrome, vasculitis, systemic vasculitis, temporal arteritis, atherosclerosis, sarcoidosis, myasthenia gravis, autoimmune hemolytic anemia (immune pancytopenia, paroxysmal nocturnal hemoglobinuria), pernicious anemia, autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis, autoimmune oophiritis), autoimmune orchitis, autoimmune uveitis, anti-phospholipid syndrome, dernyelinatiruz diseases of the central and peripheral nervous systems such as multiple sclerosis, idiopathic demyelinating polyneuropathy or Guillain-Barre syndrome, and chronic inflammatory demyelinating polyneuropathy, hepatobiliary diseases such as infectious hepatitis (hepatitis A, B. C. I). E and other non-hepatotropic viruses), autoimmune chronic active hepatitis, viral hepatitis, primary binary cirrhosis, granulomatous hepatitis, Wegener's granulomatosis, Behcet's disease, and sclerosing cholangitis, inflammatory bowel diseases such as ulcerative colitis or Crohn's disease, celiac disease, gluten-sensitive enteropathy, and Whipple's disease, autoimmune or immune-mediated skin diseases including bullous skin diseases, erythema multiforme and contact dermatitis, dermitis herpetiformis, psoriasis, pemphigus vulgaris, vitiligo (leukoderma), allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, chronic obstructive pulmonary disease, and transplantation associated diseases including graft rejection and graft-versus-host-disease. For example, in one aspect, the anti-NKG2D antibody is used in combination with one or more other anti-inflammatory agents, including, but not limited to, analgesic agents, immunosuppressive agents (e.g., B- or T-cell antagonists such as B-cell depletion agents and T cell inhibiting agents; complement inhibiting agents), corticosteroids, and anti-TNFalpha agents or other anti-cytokine or anti-cytokine receptor agents, and anti-angiogenic agents. Specific examples include tnetothrexate, TSG-6, Rituxan® or other B-cell therapies, anti-IL12 (p410) antibodies, CTLA4-Fc fusion proteins, IL-1-receptor antagonists, IL-1 antibodies, IL-15 antibodies, IL-18 antibodies, and anti-IL6R antibodies. Further examples of combination therapies are provided below.

When one or more other agents or approaches are used in combination with the present therapy, there is no requirement for the combined results to be additive of the effects observed when each treatment is conducted separately. Although at least additive effects are generally desirable, any decrease in NKG2D activity or other beneficial effect above one of the single therapies would be of benefit. Also, there is no particular requirement for the combined treatment to exhibit synergistic effects, although this is certainly possible and advantageous. The NKG2D-based treatment may precede, or follow, the other treatment by, e.g., intervals ranging from minutes to weeks and months. It also is envisioned that more than one administration of either the anti-NKG2D composition or the other agent will be utilized. The agents may be administered interchangeably, on alternate days or weeks; or a cycle of anti-NKG2D treatment may be given, followed by a cycle of the other agent therapy. In any event, all that is required is to deliver both agents in a combined amount effective to exert a therapeutically beneficial effect, irrespective of the times for administration.

### Dosages

For administration of the antibody, the dosage ranges from about 0.0001 to 100 mg/kg, and more usually 0.01 to 5 mg/kg, of the host body weight. For example, dosages can be about 0,3 mg/kg body weight, about 1 mg/kg body weight, about 3 mg/kg body weight, about 5 mg/kg body weight or about 10 mg/kg body weight or within the range of 1-10 mg/kg. An exemplary treatment regime entails administration twice per week, once per week, once every two weeks, once every three weeks, once every four weeks, once a month, once every 3 months or once every three to 6 months. Preferred dosage regimens for an anti-hNKG2D antibody of the invention include about 1, 3 or 10 mg/kg body weight body weight via intravenous administration or subcutaneous injection, with the antibody being given using one of the following dosing schedules: (i) loading doses every 1-3 weeks for 2-4 dosages, then every two; months (ii) every four weeks; (iii) every week, or any other optimal dosing. In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml. Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or non-prophylactic (e.g., palliative or curative). In prophylactic applications, a relatively low dosage is administered at relatively infrequent intervals over a long period of time. Some patients continue to receive treatment for the rest of their lives. In palliative or curative applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

The appropriate doses of anti-inflammatory agents will approximate those already employed in clinical therapies Wherein the anti-inflammatory agents are administered alone or in combination with other agents. Variation in dosage will likely occur depending on the condition being treated. The physician administering treatment will be able to determine the appropriate dose for the individual subject.

### Articles of Manufacture

In another embodiment of the invention, an article of manufacture containing materials useful for the treatment of the disorders described above is provided. For example, the article of manufacture can comprise a container containing a human or humanized anti-hNKG2D antibody as described herein together with instructions directing a user to treat a disorder such as an autoimmune or inflammatory disease or disorder in a human with the antibody in an effective amount. The article of manufacture typically comprises a container and a label or package insert on or associated with the container. Suitable containers include, for example, bottles, vials, syringes, etc. The containers may be formed from a variety of materials such as glass or plastic. The container holds a composition that is effective for treating the condition and may have a sterile access port (tier example, the container may be an intravenous solution bag or a vial having a stopper pierceable by a hypodermic injection needle). At least one active agent in the composition is the human or humanized anti-hNKG2D antibody herein, or an antigen-binding fragment or antibody derivative (e.g., an immunoconjugate) comprising such an antibody. The label or package insert indicates that the composition is used for treating the condition of choice, such as, e.g., rheumatoid arthritis.

Moreover, the article of manufacture may comprise (a) a first container with a composition contained therein, wherein the composition comprises the human or humanized antibody herein, and (b) a second container with a composition contained therein, wherein the composition comprises a therapeutic agent other than the human or humanized antibody. The article of manufacture in this embodiment of the invention may further comprise a package insert indicating that the first and second compositions can be used in combination to treat an autoimmune or inflammatory disease or disorder. Such therapeutic agents may be any of the adjunct therapies described in the preceding section. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

Further details of the invention are illustrated by the following non-limiting Examples.

### Example 1

### Generation and Initial Screening of Human Monoclonal Antibodies Against hNKG2D

### Materials and Methods

### Antigen.

Soluble NKG2D-hFc fusion protein (R&D, cat: 1299-NK) or NKG2D expressed on the surface of cells (NK, BAF, or CHO) were used as antigens for immunization. The BAF cells were co-transfected with frill-length NKG2D and DAP10. The CHO cells were transfected with an NKG2D point mutant that transports to the cell surface without DAP10 (Wu et al., Science 1999; 385:730-2). The NK cells were primary NK cells naturally expressing NKG2D.

### Mice.

Fully human monoclonal antibodies against NKG2D were produced in the KM Mouse™ strain of transgenic mice that express human antibody genes (PCT publication WO 02/43478 to ishida et al.). In this mouse strain, the endogenous kappa light chain gene has been homozygously disrupted as described in Chen et al (1993) EMBO J. 12:811-820, and the endogenous mouse heavy chain has been homozygously disrupted as described in Example 1 of PCT Publication WO 01/09187 for Humab mice. The mouse strain carries a human kappa light chain transgene, KC05, as described in Fishwild et al (1996) Nature Biotechnology 14:845-851. The mouse strain also carries a human heavy chain transchromosome, SC20, as described in WO0243478.

### Immunizations.

In a first series of immunizations, animals were immunized intraperitoneally with alternating injections of NKG2D-transfected BAF cells and NKG2D-transfected CHO cells, or primary human NK cells with or without any adjuvant. Each mouse was immunized IP with 5×10⁶ cells every or every other week (6 times in total). The mice were boosted with 5×10⁶ NKG2D-transfected BAF cells intravenously 3 and 2 days before sacrifice and removal of the spleen. The animal experiments were performed according to Danish National Research Council imidelines.

In a second series of immunizations, animals were immunized intraperitoneally and in the foot path with NKG2D-hFc with different adjuvant. Each mouse was immunized 7×25 μg NKG2F-hFc/Ribi/ip/sc, 1×25 μg NKG2D-hFc/CFA/ip/sc, 1×25 μg NKG2DhFc/IFA/ip/sc, 1×30 μg anti-CTLA4+40 ug NKG2D-hFc/IFA/ip/sc, 1×25 ug NKG2DhFc/Ribi/ip/sc and boosted 2×30 ug;/PBS/ip/iv 3 and 2 days before sacrifice and removal of the spleen. The animal experiments were performed according to American National Research Council guidelines.

### Screening of Mouse Sera.

The sera from the immunized mice were screened by flow cytometry analysis for NKG2D-specificity and selected sera were also tested for their ability to neutralize binding of the MICA ligand, as described in Example 3. Mice that had generated high titers of antibodies that specifically bound NKG2D and neutralized MICA binding were selected for hybridoma production.

### Generation of Hyridomas.

The spleen from each selected immunized mouse was homogenised and a single cell suspension of splenocytes used for fusion to X61 AgS653 myeloma cells (ATCC, CRL 1580). The fusions were performed using polyethyleneglycol (PEG) 1500 as previously described (Harlow and Lane, ANTIBODIES: A LABORATORY MANUAL, Cold Spring Harbor Laboratory Press, Cold Spring Harbor, N.Y., (1988)) and electrofusion using the The Cyto Pulse™ CEEF-50 Electrofusion System (Cyto Pulse Sciences, Inc.).

The fused cells were initially seeded in 96-well tissue culture plates in selective DMEM HAT medium, supplemented with 10% FBS and 5% origin (Hybridoma cloning Factor, BioVeris). The plates were incubated for 10-14 days with 1-2 medium changes, respectively, to DMEM HT medium supplemented with 5% FBS and 0.7% origin, before harvest and screening of the supernatants. Clones tested positive were expanded and subcloned by limiting dilution until stable clones had been generated. The selected clones were continuously screened for the presence of anti-NKG2D specific antibodies by FACS analysis as well as for their ability to neutralize MICA binding.

### Screening of Hybridoma Supernatants.

The primary screening of the hybridoma supernatants from the first series of immunizations was performed using direct ELISA or flow cytometry analysis (FACS) to test for the presence of anti-NKG2D specific antibodies. Briefly, the ELISA was performed by coating maxisorp plates with 50 μl 0 0.4 μg/ml triFc-NKG2D (comprising the extracellular portion of NKG2D fused to murine Fc and expressed in CHO cells) overnight in PBS at 4° C., followed by blocking with PBS, 0.05% Tween 20, for 15 min at room temperature. The plates were subsequently incubated with 50 μl hybridoma supernatant, and NKG2D-specific antibodies detected using Goat-Anti-human IgG-HRP Fcγ Fragment specific (Jackson, 109-036-098). These incubations were performed for 1 hr at room temperature, and between each step the plates were washed with PBS, 0.05% Tween 20. Bound antibodies were visualized using 100 μl TMB substrate (Kem-En-Tec), and stopped with 4M H₃PO₄. The plates were read at 450 and 620 nm. For FACS, binding to NKG2D-expressing BaF/3 cells and control BaF/3 cells not expressing NKG2D was analyzed by incubation of 50000 cells in 10 μl with 90 μl hybridoma supernatant for 30 min at 4° C., followed by washing with PBS with 2% FCS, and subsequently incubated with secondary Goat-AMi-human IgG-HRP Fcγ Fragment specific (Jackson, 109-036-098). The cells were then analysed on a B&D FACSArray (BD Biosciences). Antibodies that only stained NKG2D-expressing BaF/3 cells and not control cells were deemed NKG2D-specific.

The primary screen for the second series of immunizations was a direct ELISA to test for the presence of anti-NKG2D specific antibodies. Briefly, the ELISA was performed by coating maxisorp plates with 1-2 mg/ml hFc-NKG2D (R&D Systems) overnight in PBS at 4° C., followed by blocking with PBS, 0.05% Tween 20, 5% chicken serum for 30-60 min at room temperature. The plates were subsequently incubated with 50 μl hybridoma supernatant and 50 μl blocking butler, and NKG2D-specific antibodies detected using Anti-human IgG-FIRP (Bethyl, A80-115P) in blocking buffer. These incubations were performed for 1 hr at room temperature, and between each step the plates were washed with PBS, 0.05% Tween 20. Bound antibodies were visualized using ABTS substrate (Moss Inc, product: ABTS-1000). The plates were read at 415 nm with Molecular Devices Software. Hybridomas selected from an ELISA primary screen were subjected to a secondary screen using FACS, as described above. Commercially available murine antibodies (149810 and ON72) were used as controls.

### Results

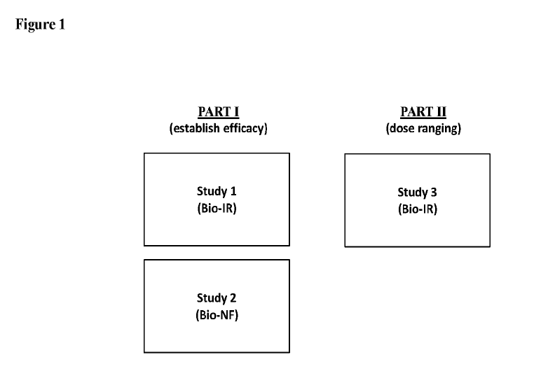

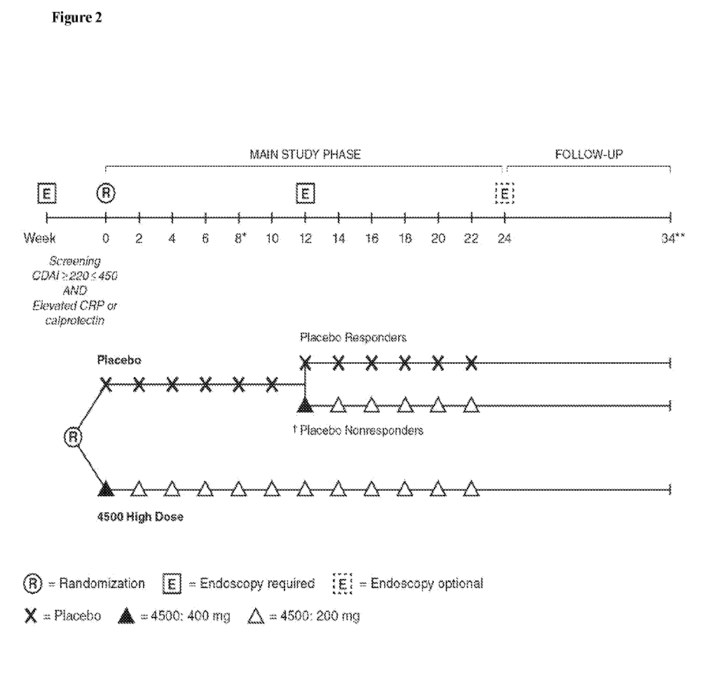

Highly selective sera from immunized mice were identified by NKG2D-binding and ligand blocking ability (exemplary results shown in FIGS. 1A and 1B), and selected mice were used for fusion and hybridoma generation. About 2500 hybridomas were screened by HASA and flow cytometry and NKG2D-.specific clones identified. FIG. 2 shows that human antibody in a hybridoma supernatant bound to NKG2D-expressing cells but not NKG2D-negative cells, comparing to a commercial antibody (149810). Antibodies from three hybridomas (16F16, 16F31 and 21F2) from the first series of immunization, and several antibodies from the second series of immunizations (including MS), were selected for recombinant production and further testing.

### Example 2

### Recombinant Production and Sequencing

A second batch of several hundreds of hybridomas from fusions mice spleens expressing human antibodies were obtained from a separate round of immunization(s). These were screened for NKG2D-specificity using FACS in the same manner as described in Example 1. Antibodies from one hybridoma, MS, were selected for recombinant production and further testing.

The variable regions of the heavy and light chains of the antibodies were identified by PCR and subsequent sequencing of the isolated product, of mRNA from the hybridoma.

### Materials and Methods

### RNA Purification.

Total RNA was purified using RNeasy from Qiagen according to the manufactures instructions, except that ii-mercaptoethanol was omitted from the procedure. The quality of the RNA was checked by light spectroscopy (260/280 nm, 1.8<ratio<2.0) and occasionally RNA degradation was evaluated using a bioanalyser.

### RT-PCR.

Full length cDNA was synthesised by SMART-RACE (kit from Clonetech) PCR.

PCR was performed with the HFII polymerase from Clonetech. The 5′ primer (with EcoRI) annealed to a conserved sequence introduced during SMART-RACE. Two 3′ primers were designed that anneal to conserved regions of the IgG (VH) and kappa chains (VL), respectively. Restriction sites were also present in the 3′ primers (BsiWI (VL) and Nhel (VH)), The PCR was performed in duplicate (to check for PCR introduced mutations) for all VH and VL amplifications. If the PCR reaction failed, the VL and VH were amplified using a degenerate 5′ primer mix from Novagen.

### PCR Product Purification.

The PCR product (^{˜}550 bp) was separated on a 1% agarose gel,excised, purified on GFX columns (from Amersham) and eluted in DNAse free water.

### Ligation.

The PCR products and the expression vector (ampicillin resistance) were cut with appropriate restriction enzymes (VH, EcoRI+Nhel and VL, EcoRI+BsiWI). The ligation of the variable domains into the isotype-dictating vector (IgG4 for NKG2D) was catalyzed by the T4-ligase (Roche). The plasmid used was pTT5 (Durocher et al., Nucleic Acids Res 2002; 30 (2):e9; Pham et al., Biotechnol Bioeng 2003; 84 (3):332-42).

### Check of Insert in the Expression Vector (Colony PCR).

Competent E. coli (Top10) were transformed with the ligation mix and ampicillin resistant clones were selected overnight. In total, 8 positive colonies for both VH and VL were picked. Via colony PCR and gel electrophoresis (1% agarose), all colonies were checked for inserts matching the expected size.

### Sequencing/Miniprep.

An aliquot from all positive colony PCRs was prepared for sequencing (using ExoSAPit). In total, 32 PCR products were sequenced for each clone ((8*VH+8*VL)*2 (PCR in duplicate)). The sequences were analysed (using VectorNTI) and positive bacteria clones corresponding to the cloned VII and VL were up-scaled (mini/maxiprep), and the DNA purified for HEK293/6E transfection (GFX columns). If more than one VH and VL sequence was identified, then all possible VL and VH combinations were expressed in HEK293/6E cells.

### Recombinant Production.

The identified variable regions of heavy and light chains were inserted into heavy and light chain human IgG4 framework respectively and expressed from two vectors in HEK293 cells at a high level. The antibodies were purified on a protein A column.

### Antibody Expression in HEK293/6E Cells.

HEK293 cells were passaged in Freestyle293 medium from Gibco. On the day of transfection, cells were diluted to a concentration of l million cells/ml. For a 30 ml transfection, 15 μg of heavy-chain vector and 15 μtg of light-chain vector were mixed with 2 ml Opti-MEM and 40 μl 293fectin (then Freestyle293 medium to a total volume of 30 ml). After 6 days of incubation, cells were pelleted by centrifugation (1000 rpm, 10 min) and the supernatant was harvested for protein A purification.

### Purification.

The recombinantly expressed IgG4 variants of the human antibodies was purified on MabSelect™ SuRe protein-.A columns. After column application of antibody, the column was washed with 10 column volumes of PBS buffer, and antibody eluted with 100 mM Glycine, 100 mM NaCl buffer. pH 3.0, followed by buffer exchange into PBS buffer using a HighTrap™ Desalting column. All operations were controlled by an Aktaxpress system from GE Healthcare Amersham Biosciences AB. The typical concentration range of purified antibody was from 10-130 mg/l (0.3-3.3 mg/30 ml).

### Results

cDNA sequences encoding 16E16 (IgG4) H chain, 16F16 L chain, 16F31 (1gG4) H chain, and 16F31 L chain are disclosed in U.S. Pat. No. 7,879,985 incorporated herein by reference, and respective sequence identifiers of full-length, variable, and CDR amino acid sequences of 16F16 (IgG4). 16F31 (IgG4), MS (IgG4) and 21F2 (IgG4) are disclosed in U.S. Pat. No. 7,879,985 incorporated herein by reference.

An exemplary anti-NKG2D antibody that was generated using these procedures and will be used in the clinical protocol described herein comprises a VU region and a VL region with the sequences set forth below:

Anti-NKG2D VH
(SEQ ID NO: 1)
QVHLQESGPGLVKPSETLSLTCTVSDDSISSYYWSWIRQPPGKGLEWIGH
ISYSGSANYNPSLKSRVTISVDTSKNQFSLKLSSVTAADTAVYYCANWDD
AFNIWGQGTMVTVSS
Anti-NKG2D VL
(SEQ ID NO: 2)
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK

An exemplary anti-NKG2D antibody that was generated using these procedures and will be used in the clinical protocol described herein comprises heavy chain CDR amino acid sequences of SEQ ID NO: 3, SEQ ID NO: 4, and SEQ ID NO: 5 and light chain CDR amino acid sequences of SEQ ID NO: 6, SEQ ID NO: 7, and SEQ ID NO: 8.

SEQ ID NO: 3
SYYWS
SEQ ID NO: 4
HISYSGSANYNPSLKS
SEQ ID NO: 5
WDDAFNI
SEQ ID NO: 6
RASQSVSSSYLA
SEQ ID NO: 7
GASSRAT
SEQ ID NO: 8
QQYGSSPWT

### Example 3

### Anti-NKG2D Antibody for the Treatment of Moderately to Severely Active Crohn's Disease

### Introduction

This clinical proof of principal trial will provide information about the safety and of an anti-NKG2D antibody in participants with moderately to severely Crohn's Disease.

### Features of the Anti-NKG2D to be Used in the Study

A human immunoglobulin G4 isotype monoclonal antibody that binds specifically to the natural killer group 2 member D (NKG2D) receptor, comprising VH and VL regions set forth in SEQ ID NO:1 and SEQ ID NO: 2, respectively, will be used in these studies. This antibody blocks NKG2D ligand binding, thereby preventing the downstream-signaling events that otherwise lead to cell proliferation and release of proinflammatory cytokines and cytotoxic mediators. Several lines of evidence from patients with Crohn's disease support the hypothesis that NKG2D receptor activation plays a role in disease pathogenesis by mediating the production of local cytokines, activation of an immune response, and direct cytotoxicity of target intestinal cells. Collectively, preclinical and clinical data on the expression of NKG2D ligands or proinflammatory cytokines in the target tissue and abnormal expression and activation of the NKG2D receptor on CD8+ and CD4+ T cells provide a rationale for the clinical development of inhibitors of the NKG2D receptor.

### Clinical Studies

As of 11 Nov. 2015, a total of 105 subjects had been exposed to an anti-NKG2D antibody in 3 clinical studies: 65 subjects in 2 studies in rheumatoid arthritis (RA) and 40 subjects in a Phase 2a study in Crohn's disease.

### Rheumatoid Arthritis

Two studies with an anti-NKG2D antibody were conducted in subjects with active RA. In a first-in-humans (FIH), Phase 1, single ascending dose/multiple ascending dose study that included single-dose (0.0002 to 7.5 mg/kg) and multiple-dose (0.02 to 4 mg/kg) parts, 13 dose levels were evaluated in 24 subjects exposed to an anti-NKG2D antibody. Subcutaneous administration of an anti-NKG2D antibody was well tolerated at the dose ranges investigated and no safety signals were associated with either the single- or multiple-dose regimens. In a Phase 2a, randomized, single-dose, double-blind, placebo-controlled, parallel-group study, clinical efficacy was assessed in subjects with active RA concomitantly treated with methotrexate (MTX). A single SC injection of 4 mg/kg an anti-NKG2D antibody in 41 subjects exposed to an anti-NKG2D antibody did not result in a statistically significant reduction in disease activity at Weeks 6, 12, or 24 after treatment compared with placebo. The anti-NKG2D antibody was well tolerated and no safety concerns were raised during the study.

### Crohn's Disease

In a Phase 2a, multicenter, randomized, double-blind, placebo-controlled, parallel-group, single-dose study in subjects with moderately to severely active Crohn's disease who had failed or were intolerant to conventional therapy (corticosteroids or immunomodulators) or were intolerant or refractory to 1 TNFα antagonist therapy, only subjects who had a Crohn's Disease Activity Index (CDAI) score ≥220 but ≤450 and inflammation confirmed by C-reactive protein (CRP) ≥10 mg/dL or by endoscopy (endoscopic verification of active ulceration performed during screening and read by a blinded central imaging reader) were included in the study. The study enrolled and randomized 78 subjects at 32 investigational sites in North America, Europe, and Israel. All subjects were randomly assigned in a 1:1 ratio at Week 0 to receive placebo SC (n=38) or 2 mg/kg anti-NKG2D antibody SC (n=40). Among the 78 randomized subjects, the mean baseline CDAI score was 330.5, and 29.5% were intolerant or refractory to a maximum of 1 TNFα antagonist therapy (Matthiew A et. al. 2016 Gut 0:1-8. doi:10.1136)

Subjects were evaluated for the primary endpoint of change from baseline CDAI score at Week 4. Safety and efficacy evaluations were performed through Week 24. The observed 16-point greater reduction in CDAI in the anti-NKG2D antibody group at Week 4 compared with the placebo group was not significant (p=0.403). Based on a predefined significance level of 0.10, however, the reduction in CDAI score was significantly higher in the anti-NKG2D antibody group compared with the placebo group at Week 12 (55-point greater reduction in CDAI was observed in the anti-NKG2D antibody compared with placebo, p=0.056). Based on the same predefined significance level of 0.10, reductions in CDAI scores were significantly higher in the predefined subgroup of “no prior failure to biologics” (71% of the study population) in the anti-NKG2D antibody group compared with placebo at all post baseline visits through Week 12 (Week 1, p=0.068; Week 2, p=0.048; Week 4, p=0.095; Week 8, p=0.015, Week 12, p=0.025).

As a part of this study, genetic polymorphisms in the genes for the NKG2D receptor and NKG2D ligands of subjects were evaluated. A post hoc analysis of efficacy data demonstrated greater efficacy in a subgroup of subjects with a specific single nucleotide polymorphism (SNP) in the NKG2D receptor and/or MICB ligand (SNP-positive cohort). The association between SNP-positive status and higher clinical efficacy will be tested prospectively in this Phase 2b study.

The mean duration of study participation was equivalent between the 2 treatment groups. No deaths or medical events of special interest were reported. Through Week 24, the proportions of subjects with 1 or more adverse events (AEs) were similar in the anti-NKG2D antibody and placebo groups. Gastrointestinal events were the most commonly reported AEs in both groups (17 and 14 subjects in the anti-NKG2D antibody and placebo groups, respectively). Serious AEs (SAEs) were uncommon and reported in 7 of 78 (9%) randomized subjects with 1 SAE each: 2 in the placebo group (1 Crohn's disease, 1 nephrolithiasis) compared with 5 in the anti-NKG2D antibody group (4 Crohn's disease, 1 Clostridium difficile infection). All SAEs were evaluated as unlikely related to treatment with study agent.

Collectively, these data support the further development of the anti-NKG2D antibody in subjects with moderately to severely active Crohn's disease.

This protocol is comprised of 3 separate randomized, double-blind, placebo-controlled, parallel-group, multicenter studies designed to evaluate the safety and efficacy of an anti-NKG2D antibody in subjects with moderately to severely active Crohn's disease who have previously failed or who were intolerant to 1 or more approved biologic agents (Bio-IR) or those who have demonstrated an inadequate response to or have failed to tolerate corticosteroids or immunomodulators (Bio-NF). The protocol is divided into 2 parts.

In Part I, the following 2 studies will be conducted:
Study 1: A study in subjects who are biologic intolerant or refractory (Bio-IR);
Study 2: A study in subjects who have not previously failed a biologic therapy (Biologic nonfailure [Bio-NF]).

In Part II, the following study will be conducted:
Study 3: A dose-ranging study in subjects who are biologic intolerant or refractory (Bio-IR)

The 2 studies in Part I serve to build on the original Phase 2a study findings by employing dedicated populations of both Bio-IR (biologic intolerant or refractory) and Bio-NF (those who have not previously failed a biologic therapy) subjects. If acceptable efficacy is established in the Bio-IR population in Part I, the third study of the protocol, which consists of a dose-ranging study in subjects who are biologic intolerant or refractory, will be initiated (Part II).

### Objectives and Endpoints

Objectives and Endpoints

Objectives

The objectives are the same in each of the 3 studies.

Primary Objectives
To evaluate the efficacy of an anti-NKG2D antibody to reduce the CDAI score from baseline.
To evaluate the safety of the anti-NKG2D antibody.

Secondary Objectives
To evaluate the efficacy of an anti-NKG2D antibody to induce clinical remission, clinical response, and endoscopic healing of the mucosa, and to maintain remission
To evaluate the relationship between efficacy and the presence of the NKG2D and/or MICB SNP biomarkers.
To evaluate the efficacy of an anti-NKG2D antibody to improve general and disease-specific health-related quality of life and to reduce Crohn's disease-related hospitalizations and surgeries.
To evaluate the pharmacokinetics, immunogenicity, pharmacodynamics, and biomarkers (e.g., reductions in CRP, fecal calprotectin, and fecal lactoferrin) of an anti-NKG2D antibody therapy.

Endpoints

The primary endpoint for each of the 3 studies is: Change from baseline in the CDAI score at Week 8.

The following endpoints will be evaluated as major secondary endpoints only in Study 3 (the dose-ranging portion of the study); these endpoints will be evaluated in Study 1 and Study 2, but are not specified as major secondary endpoints.
Clinical remission at Week 8 as measured by CDAI (CDAI <150).
Clinical response at Week 8 as measured by CDAI (≥100-point reduction from baseline in CDAI or CDAI <150).
Change in PRO-2 (the sum of the abdominal pain and stool frequency subscores of the CDAI score) from baseline at Week 8.
Clinical remission at Week 8 as measured by PRO-2 (PRO-2 <75).
Clinical response at Week 8 as measured by PRO-2 (≥50-point reduction from baseline in PRO-2 or PRO-2 <75).
Change in Simple Endoscopic Score for Crohn's Disease (SES-CD) from baseline at Week 12.

The following efficacy endpoints will be evaluated in each of the 3 studies:
Change in CDAI from baseline at all postbaseline visits.
Clinical remission based on CDAI at all postbaseline visits.
Clinical response based on CDAI at all postbaseline visits.
Change in PRO-2 from baseline at all postbaseline visits.
Change in abdominal pain score (mean daily average based on the CDAI assessment) from baseline at all postbaseline visits.
Change in stool frequency score (mean daily average based on the CDAI assessment) from baseline at all postbaseline visits.
Clinical remission based on PRO-2 at all postbaseline visits.
Clinical response based on PRO-2 at all postbaseline visits.
Change in PRO-3 (the sum of abdominal pain, stool frequency, and general well-being subscores of the CDAI score) from baseline at all postbaseline visits.
Clinical remission based on CDAI at Week 24 among subjects in clinical response at Week 8.
Clinical remission based on CDAI at Week 24 among subjects in clinical remission at Week 8.
Change in SES-CD score from baseline at Weeks 12 and 24.
Endoscopic improvement at Weeks 12 and 24 based on a reduction from baseline in SES-CD score ≥3.
At least 50% improvement from baseline in SES-CD at Weeks 12 and 24.
Endoscopic healing (defined as the absence of mucosal ulcerations) at Weeks 12 and 24.
Fistula response at all postbaseline visits, defined as a ≥50% reduction from baseline in the number of draining fistulas.
Endpoint(s) based on Bristol stool form scale (to be detailed in the Statistical Analysis Plan [SAP]).
Change in abdominal pain from baseline at all postbaseline visits based on a 0-10 Numerical Rating Scale (NRS).
Change in Inflammatory Bowel Disease Questionnaire (IBDQ) score from baseline at Weeks 8, 12, and 24.
Clinical remission based on IBDQ (≥170) at Weeks 8, 12, and 24.
A ≥16-point improvement in IBDQ from baseline at Weeks 8, 12, and 24.
Change from baseline in the Physical Component Summary (PCS) and Mental Component Summary (MCS) scores of the 36-item Short Form Health Survey (SF-36) at Weeks 8, 12, and 24.
A ≥5-point improvement in PCS or MCS scores of the SF-36 at Weeks 8, 12, and 24.
Change in biomarkers (CRP, fecal calprotectin, fecal lactoferrin) from baseline at Weeks 8, 12, and 24.
Clinical remission based on CDAI at Week 8 by SNP status. Subjects who are positive in at least 1 of 2 SNPs (NKG2D or MICB) will be considered to be SNP-positive.
Other efficacy endpoints may be examined by SNP status (to be detailed in the SAP).

Refer to Section 0, Study Evaluations, for evaluations related to endpoints.

### Example 4

### Study Design and Rationale

### Overview of Study Design

This protocol is comprised of 3 separate studies conducted in 2 parts (FIG. 1) that are designed to evaluate the safety and efficacy of an anti-NKG2D antibody in subjects with moderately to severely active Crohn's disease.

In Part I, the following 2 studies will be conducted:
Study 1: A study in subjects who are biologic intolerant or refractory (Bio-IR);
Study 2: A study in subjects who have not previously failed a biologic therapy (Biologic nonfailure [Bio-NF]).

In Part II, the following study will be conducted:
Study 3: A dose-ranging study in subjects who are biologic intolerant or refractory (Bio-IR)

The 2 studies in Part I serve to build on the original Phase 2a study findings by employing dedicated populations of both Bio-IR (biologic intolerant or refractory) and Bio-NF (those who have not previously failed a biologic therapy) subjects. If acceptable efficacy is established in the Bio-IR population, the third study of the protocol, which consists of a dose-ranging study in subjects who are biologic intolerant or refractory, will be initiated (Part II) (see FIG. 1).

### Example 5

### Participants

The target population for each of the studies consists of men or women ≥18 years of age with moderately to severely active Crohn's disease (of at least 3 months' duration), defined as a CDAI score ≥220 but ≤450 at Week 0, with elevated CRP >0.3 mg/dL (>3.0 mg/L) and/or calprotectin >250 mg/kg at screening. Subjects must have colitis, ileitis, or ileocolitis previously confirmed at any time in the past by radiography, histology, and/or endoscopy.

Additionally, subjects in these studies must have previously failed or been intolerant to 1 or more approved biologic agents (i.e., TNFα-antagonists or vedolizumab, hereafter referred to as biologic intolerant or refractory subjects) or have demonstrated an inadequate response to or failed to tolerate corticosteroids or immunomodulators (i.e., 6-mercaptopurine [6-MP], azathioprine [AZA], and MTX) but not a biologic agent (hereafter referred to as biologic nonfailure subjects). These two populations are described below:
Biologic intolerant or refractory (Bio-IR) subjects (Study 1 and Study 3) are defined as those who have received infliximab (or a biosimilar for infliximab), adalimumab (or a biosimilar for adalimumab), certolizumab pegol, or vedolizumab at a dose approved for the treatment of Crohn's disease, and either did not respond initially, responded initially but then lost response, or were intolerant to the medication. Bio-IR subjects must allow a ≥8-week washout for prior TNFα antagonist use and a 16-week washout period for prior vedolizumab use.
Biologic nonfailure (Bio-NF) subjects (Study 2) are defined as those who have demonstrated an inadequate response to or have failed to tolerate corticosteroids or the immunomodulators 6-MP, AZA, or MTX. Subjects who have demonstrated corticosteroid dependence (i.e., an inability to successfully taper corticosteroids without a return of the symptoms of Crohn's disease) are also eligible. Bio-NF subjects may also have received biologic therapy but only if it was discontinued for reasons other than lack of efficacy or intolerance (eg, drug holiday).

It is anticipated that approximately 450 subjects will be enrolled overall across the three studies:
Part I will study the safety and efficacy of a high-dose regimen of an anti-NKG2D antibody compared with placebo and will enroll approximately 200 subjects (100 Bio-IR and 100 Bio-NF).
Part II will study the safety and efficacy of multiple dose regimens of anti-NKG2D antibody compared with placebo, with ustekinumab (STELARA®) as a reference arm. Part II will enroll approximately 250 additional subjects; all subjects enrolled in Part II will be Bio-IR subjects.
Schematic representations of Part I and Part II are shown in FIG. 2 and FIG. 3, respectively.

Throughout both parts of the study, efficacy, PK, PD, immunogenicity, biomarkers, and safety will be assessed at timepoints indicated in the appropriate Time and Events Schedules.

Blood samples for pharmacogenomic analyses will be collected from subjects who consent separately to this component of the protocol (where local regulations permit). Subject participation in pharmacogenomic research is optional.

Each of the three studies will be analyzed separately. The primary endpoint for each study is the change from baseline in the CDAI score at Week 8.

An external Data Monitoring Committee (DMC) will review unblinded safety data from the 3 studies periodically to monitor subject safety. The DMC will consist of at least one medical expert in the relevant therapeutic area and at least one statistician. The DMC responsibilities, authorities, and procedures will be documented in its charter.

### Example 6

### Part I

In Part I, 100 Bio-IR subjects and 100 Bio-NF subjects will be randomly assigned to receive placebo or the anti-NKG2D antibody high dose in a 1:1 ratio using permuted block randomization, stratified by baseline CDAI score (≤300 or >300) and SNP-positive status (yes or no). Separate randomizations will be used for the Bio-IR and Bio-NF populations. In order to have a sufficient number of SNP-positive subjects in each of the populations, more than 50 subjects per group might be randomized if the proportions of SNP-positive subjects in Study 1 or Study 2 are less than 75% (assumed prevalence).

The treatment groups for each study in Part I will be as follows:
Placebo SC at Weeks 0, 2, 4, 6, 8, and 10; from Week 12, these subjects will receive additional doses as follows:
   Placebo-treated subjects who are in clinical response at Week 12 (≥100-point reduction from baseline in CDAI or CDAI <150) will continue to receive placebo SC injections q2w from Week 12 through Week 22.
   Placebo-treated subjects who are not in clinical response at Week 12 will receive anti-NKG2D antibody 400 mg SC at Week 12 and then anti-NKG2D antibody 200 mg SC q2w from Week 14 through Week 22.
anti-NKG2D antibody 400 mg SC at Week 0 then 200 mg SC q2w through Week 22.

An interim analysis is planned in Part I when the first 80% of the randomized Bio-IR subjects in Study 1 (at least 40 Bio-IR subjects and at least 30 Bio-IR/SNP-positive subjects per treatment group) have completed their Week 8 visit or have terminated their study participation before Week 8.

The interim analysis may allow for an earlier start of Part II. Enrollment of Part I Bio-IR subjects will continue until 100 Bio-IR subjects have enrolled regardless of whether Part II is started early. If the decision is made to start Part II based on the interim analysis, enrollment of Bio-NF subjects in Part I will continue until 100 Bio-NF subjects have been enrolled. If Part II was not initiated based on the interim analysis, data will be analyzed when all Bio-IR subjects have completed their Week 12 visit (or terminated study participation prior to Week 12) to determine whether or not to move to Part II.

### Example 7

### Part II

In Part II, 250 additional Bio-IR subjects will be randomly assigned to receive placebo or 1 of 3 dose levels of the anti-NKG2D antibody or ustekinumab in a ratio of 1:1:1:1:1 using permuted block randomization, stratified by baseline CDAI score (≤300 or >300) and SNP-positive status (yes or no). In order to have a sufficient number of SNP-positive subjects, more than 50 subjects per group might be randomized if the proportion of SNP-positive subjects in Study 3 is less than 75% (assumed prevalence).

The treatment groups in Part II will be as follows:
Placebo SC at Weeks 0, 2, 4, and 8; from Week 12, these subjects will receive additional doses as follows:
   Placebo-treated subjects who are in clinical response at Week 12 (≥100-point reduction from baseline in CDAI or CDAI <150) will continue to receive placebo at Weeks 12, 14, 16, and 20.
   Placebo-treated subjects who are not in clinical response at Week 12 will receive anti-NKG2D antibody 150 mg SC at Week 12 and then anti-NKG2D antibody 75 mg SC at Weeks 14, 16, and 20.
High dose: anti-NKG2D antibody 400 mg SC at Week 0 and 200 mg SC at Weeks 2 and 4, then 200 mg SC every 4 weeks (q4w) through Week 20.
Middle dose: anti-NKG2D antibody 150 mg SC at Week 0 and 75 mg SC at Weeks 2 and 4, then 75 mg SC q4w through Week 20.
Low dose: anti-NKG2D antibody 50 mg SC at Week 0 and 25 mg SC at Weeks 2 and 4, then 25 mg SC q4w through Week 20.
Ustekinumab (tiered doses approximating 6 mg/kg IV) at Week 0 (as indicated in the bullets below), followed by 90 mg SC at Weeks 8 and 16.
   Ustekinumab 260 mg (weight <55 kg).
   Ustekinumab 390 mg (weight >55 kg and ≤85 kg).
   Ustekinumab 520 mg (weight >85 kg);

As indicated in FIG. 2, subjects will also receive placebo administrations, as necessary, to maintain the blind of Part II.

### Example 8

### Interim Analysis

An interim analysis is planned in Part I when the first 80% of the randomized Bio-IR subjects in Study 1 (at least 40 Bio-IR subjects and at least 30 Bio-IR/SNP-positive subjects per treatment group) have completed their Week 8 visit or have terminated their study participation before Week 8.

This interim analysis will allow for an earlier start of Part II (i.e., Study 3, the dose-ranging part) if the results suggest that a sufficient number of subjects have been evaluated for the purpose of demonstrating effect. As this interim analysis does not affect the conduct or completion of Study 1, it will be considered administrative and will not require multiplicity adjustment for the final Study 1 analysis.

A sponsor committee independent of the study team will be established to review the interim data and formulate recommended decisions/actions in accordance with predefined decision rules (to be defined in the Interim Analysis Plan).

An interim analysis is not planned for Study 2 or Study 3.

### Example 9

### Study Design Rationale

This protocol is comprised of 3 separate studies conducted in 2 parts that are designed to evaluate the safety and efficacy of anti-NKG2D antibody in subjects with moderately to severely active Crohn's disease. Study 1 and Study 2 constitute Part I of the protocol. In this part, the safety and efficacy of a single dosing regimen of the anti-NKG2D antibody in Bio-IR and Bio-NF subjects with moderately to severely active Crohn's disease is evaluated. If acceptable efficacy is established in Part I (for the Bio-IR subjects), Part II (a dose ranging study in Bio-IR subjects) will be initiated.

### Study Population

The target population for each of the 3 studies consists of men or women ≥18 years of age at the time of informed consent with moderately to severely active Crohn's disease (of at least 3 months' duration), defined as a CDAI score ≥220 and ≤450, with elevated CRP >0.3 mg/dL (>3.0 mg/L) and/or calprotectin >250 mg/kg.

The Bio-IR population, comprising subjects who have failed to respond, lost response, or have been intolerant to one or more biologic therapies, is the primary population of interest for this protocol because it has the highest unmet need with current therapies. Responses to therapies are generally lower in the Bio-IR population than in the Bio-NF population.

The cohort of Bio-NF subjects is included in Part I (Study 2) to obtain additional information about the effect of the anti-NKG2D antibody in this population early in the development program.

### Choice of the Anti-NKG2D Antibody Dose for Placebo Nonresponders

In Part 1, the high dose of the anti-NKG2D antibody (400 mg/200 mg) was chosen for placebo nonresponders because it is the only dose regimen studied in this part.

In Part 2, the middle dose was chosen for placebo nonresponders as it is believed that this dose will be effective since it is higher than the dose studied in the prior Phase 2a study (where efficacy was shown). The middle dose also requires fewer injections compared with the high dose.

### Example 10

### Assessments

### Efficacy Assessments

The efficacy evaluations selected for both parts of the study (e.g., CDAI, CRP, fecal biomarkers;) are well-established measures that are accepted by regulatory agencies as primary or supportive of clinically relevant effect of disease activity in Crohn's disease studies.

CDAI will be calculated at the final efficacy and safety visit to evaluate the level of efficacy after prolonged discontinuation of study drug.

Change in the CDAI is being used as the primary endpoint for each of the 3 studies because this measure is more sensitive than remission (i.e., the change in CDAI provides greater power than remission for the same sample size). Therefore, the study can be more efficient for Phase 2 using the change in CDAI. The clinical remission endpoint is being used for the interim analysis, however, as it is a more stringent endpoint and provides a more conservative decision rule to determine whether to start Part II early.

Because it is anticipated that endoscopic improvement will occur later than the clinical symptoms (e.g., change in CDAI), the initial assessment of endoscopy improvement will occur at Week 12 (instead of Week 8). In order to have an appropriate comparison of the anti-NKG2D antibody to placebo, the placebo-controlled period will continue to Week 12.

### Pharmacokinetic Assessments

Pharmacokinetic assessments will be used to further understand the disposition of the anti-NKG2D antibody in subjects with Crohn's disease.

### Immunogenicity Assessments

Serum samples for the detection of antibodies to the anti-NKG2D antibody will be collected to further evaluate the immunogenicity of the anti-NKG2D antibody in subjects with Crohn's disease.

### Pharmacodynamic Assessments

Serum samples for the analysis of PD will be collected to further understand the response of subjects with Crohn's disease to treatment with the anti-NKG2D antibody.

### Example 11

### DNA and Biomarker Collection

It is recognized that genetic variation can be an important contributory factor to interindividual differences in drug distribution and response and can also serve as a marker for disease susceptibility and prognosis. Pharmacogenomic research may help to explain interindividual variability in clinical outcomes and may help to identify population subgroups that respond differently to a drug.

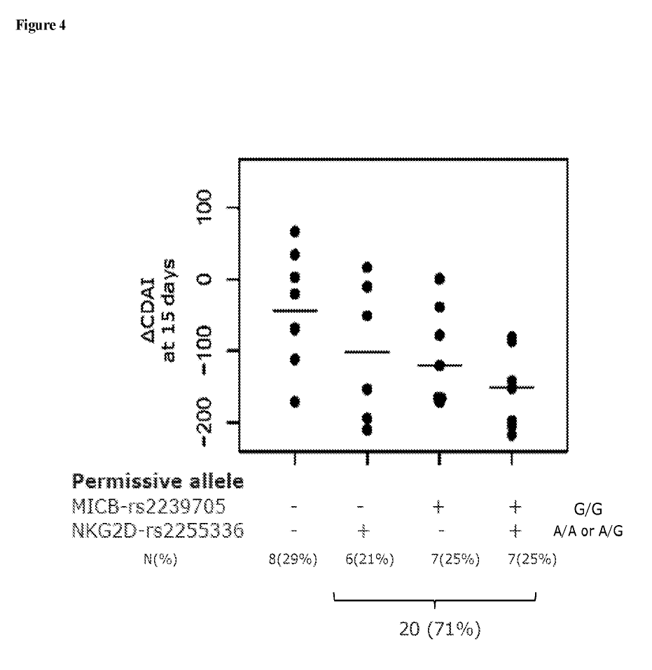

A post hoc analysis of the data from the NKG2D Phase 2a clinical trial was performed by genotyping patient samples and identifying those patients with the rs2255336 or rs2239705 SNP. Four patient groups were tested based on genotypes for the two SNPs. Subjects with all four potential haplotypes were examined. This data is summarized in FIG. 4 and shows the change in CDAI at day 15 after treatment compared to the CDAI score prior to treatment. These data indicate that the extent of reduction of CDAI phenotype following administration of anti-NKG2D antibody is correlated with the haplotype. Individuals who are compound homozygotes for the permissive alleles (i.e., harbor both the rs2255336 and rs2239705 SNP) express less MICB and NKG2D thereby conferring a greater reduction in CDAI. Conversely, individuals carrying alleles associated with higher expression of MICB and NKG2D were observed to have lower improvement in disease levels as indicated by smaller changes in CDAI. These data indicate a potential correlation between the genotype of a subject with regard to these SNPs and the clinical efficacy of the NKG2D antibody.

Whole blood will be collected from all subjects for SNP analysis (the NKG2D SNP rs2255336 and the MICB [NKG2D ligand] SNP rs2239705) to understand the association of these SNPs with response to the anti-NKG2D antibody (refer to the latest version of the IB for more information). In addition, subjects who sign an optional pharmacogenomics consent form will undergo complete genomic testing.

The goal of this pharmacogenomic component is to collect DNA to allow the identification of genetic factors that may influence the PK, PD, efficacy, safety, or tolerability of the anti-NKG2D antibody and to identify genetic factors associated with Crohn's disease.

Biomarker assessments will be made to examine the biologic response to treatment and to identify biomarkers that are relevant to the anti-NKG2D antibody treatment and/or Crohn's disease. Blood samples for serum-based biomarker analyses will be collected from all subjects to assess proteins related to the NKG2D pathway or the pathogenesis of Crohn's disease. Whole blood samples will be collected from all subjects for the analysis of RNA expression and T-cell receptor (TCR) repertoire. Mucosal biopsy samples will be collected during ileocolonoscopy for the analysis of gene and/or protein expression and the histologic assessment of disease and/or healing.

Receptor occupancy (RO) assessments for NKG2D and immunophenotyping assessments (including NK cells and CD8+ T cells) will also be performed. Immunophenotyping will be conducted using flow cytometry to assess the number of CD4, CD8, and NK cells before versus after dose administration.

### Example 12

### Control, Randomization, and Blinding

In both parts of the study, a placebo control will be used to establish the frequency and magnitude of changes in clinical endpoints that may occur in the absence of active treatment. In addition to placebo control, a ustekinumab reference arm will be used in Part II to determine the sensitivity of the clinical endpoints in this study.

Ustekinumab was chosen for use as a reference arm because the efficacy and safety profile of ustekinumab are well described. It is also recognized that use of other therapeutics (e.g., TNFα antagonists) could potentially confound the population of Bio-IR subjects and introduce substantial patient burden to maintain blinding.

Randomization will be used to minimize bias in the assignment of subjects to treatment groups, to increase the likelihood that known and unknown subject attributes (e.g., demographic and baseline characteristics) are evenly balanced across treatment groups, and to enhance the validity of statistical comparisons across treatment groups. Blinded treatment will be used to reduce potential bias during data collection and evaluation of clinical endpoints.

### Example 13

### Dose Selection

### The Anti-NKG2D Antibody

The results of PK, PD and efficacy analyses and safety data from previous clinical studies of the anti-NKG2D antibody in subjects with RA and Crohn's disease were used to inform the dose selection for the 3 studies in this protocol. In study NN8555-3618, which was the first in human clinical study with the anti-NKG2D antibody conducted in subjects with RA, the highest single SC dose investigated was 7.5 mg/kg and the highest multiple SC dose regimen investigated was 4 mg/kg every 2 weeks (q2w) for a total of 4 doses in RA subjects. The higher dose was administered in part because it was thought that more drug would be needed to get into the synovial fluid of the RA subjects. However, even at the higher dose, the anti-NKG2D antibody did not appear to be effective in these subjects. The receptor occupancy data from the previous Crohn's disease clinical study showed that receptor occupancy dropped from approximately 80% at 8 weeks to approximately <20% at week 12 (Allez M, Skolnick BE, Wisniewska-Jarosinska M, et al Anti-NKG2D monoclonal antibody (NNC0142-0002) in active Crohn's disease: a randomised controlled trial Gut Published Online First: 3 Aug. 2016. doi: 10.1136/gutjnl-2016-311824).

The anti-NKG2D antibody was well tolerated and no safety concerns were identified in subjects with RA or Crohn's disease from the previous clinical studies. In addition, a 52-week repeat-dose toxicology study has demonstrated a no-observed-adverse-effect level (NOAEL) of 100 mg/kg SC once weekly in cynomolgus monkeys. Based on these safety and toxicology findings, it is expected that the proposed dose regimens of the anti-NKG2D antibody would have acceptable safety profiles.

Population PK/PD modelling and simulation was performed using the anti-NKG2D antibody PK and NKG2D RO data from the previous clinical studies. The model-predicted anti-NKG2D antibody concentrations and NKG2D RO in the intestines were used to help guide the selection of the dose regimens for the present study. NKG2D RO in the intestines is predicted by assuming that the concentration of the anti-NKG2D antibody in the intestines is approximately 10% of the concentration in the peripheral circulation because a 5%-15% antibody distribution coefficient between the general circulation and the intestines has been reported.

### Part I

The selected anti-NKG2D antibody dose regimen for Part I includes a loading dose of 400 mg SC at Week 0, followed by 200 mg SC q2 weeks through Week 22. The loading dose of 400 mg at Week 0 is intended to produce rapid onset of clinical response. The Part I dose regimen is predicted to achieve systemic exposures similar to the maximum systemic exposure that has been well tolerated in previous clinical studies. In the first-in-human study, the highest multiple dose regimen of 4 mg/kg SC q2w (4 doses) provided a median (range) Cₘₐₓ in serum of 79.3 (52.8 to 91.2) μg/mL. The predicted median of the anti-NKG2D antibody Cₘₐₓ in serum is 64.64 μg/mL, median anti-NKG2D antibody concentration in serum at Week 8 is 38.94 μg/mL and median steady-state trough anti-NKG2D antibody serum concentration is 38.31 μg/mL in subjects with Crohn's disease (Table 2).

TABLE 2
Predicted median serum anti-NKG2D antibody concentrations
and NKG2D receptor occupancy after administration of
the selected dose regimens of anti-NKG2D antibody

| | anti-NKG2D antibody | | NKG2D Receptor | | |
|---|---|---|---|---|---|
| Dosing | Serum Concentration | | Occupancy (% RO) | | |
| Regimen | (μg/ml) | | | % RO in | % RO in |
| (SC) | Parameter | Value | Week | blood | intestine |
| 400 mg at | Cₘₐₓ | 64.64 | — | — | — |
| Week 0 | Cₘᵢₙ (Week 8) | 38.94 | 8 | 100 | 99 |
| then 200 | C_{trough} | 38.31 | 24 | 100 | 99 |
| mg q2w | (steady state) | | | | |
| 400 mg | Cₘₐₓ | 53.99 | — | — | — |
| (Wk 0), | Cₘᵢₙ (Week 8) | 18.95 | 8 | 100 | 97 |
| 200 mg | C_{trough} | 12.00 | 24 | 100 | 96 |
| (Wks 2 &4) | (steady state) | | | | |
| then 200 | | | | | |
| mg q4w | | | | | |
| 150 mg | Cₘₐₓ | 19.53 | — | — | — |
| (Wk 0), | Cₘᵢₙ (Week 8) | 6.34 | 8 | 100 | 91 |
| 75 mg | C_{trough} | 4.24 | 24 | 99 | 87 |
| (Wks 2 &4) | (steady state) | | | | |
| then 75 mg | | | | | |
| q4w | | | | | |
| 50 mg | Cₘₐₓ | 6.46 | — | — | — |
| (Wk 0), | Cₘᵢₙ (Week 8) | 1.77 | 8 | 97 | 76 |
| 25 mg | C_{trough} | 0.74 | 24 | 94 | 64 |
| (Wks 2 &4) | (steady state) | | | | |
| then 25 mg | | | | | |
| q4w | | | | | |

q2w = every 2 weeks;
q4w = every 4 weeks;
% RO = percent receptor occupancy;
SC = subcutaneous;
Wk = Week

Assuming the concentration of the anti-NKG2D antibody in the intestines is approximately 10% of concentration of the anti-NKG2D antibody in serum, the predicted median peak and trough concentrations of the anti-NKG2D antibody in the intestines are 6.46 μg/mL and 3.83 μg/mL, respectively. Analysis of the ex vivo relationship between the NKG2D RO and the anti-NKG2D antibody serum concentration suggests that ≥90% RO is achieved when the anti-NKG2D antibody serum concentration is ≥3 μg/mL. As a result, the Part I dose regimen is expected to result in approximately 99% NKG2D RO in the intestine (Error! Reference source not found.). Thus, the Part I dose regimen is expected to increase the probability to achieve maximum clinical response while remaining within the acceptable safety margins in subjects with Crohn's disease.

### Part II

Three dose regimens of the anti-NKG2D antibody (high, middle and low) have been selected for Part II which are expected to provide a wide range of systemic drug exposures in order to assess exposure-response relationship in subjects with Crohn's disease. The loading doses at Week 0, 2, and 4 are intended to produce rapid onset of clinical response. Since the apparent terminal half-life of anti-NKG2D antibody at the proposed dose regimens is about 2 to 3 weeks, SC administration of anti-NKG2D antibody at 4-week intervals from Week 4 through Week 20 is expected to produce median steady state trough serum of anti-NKG2D antibody concentrations that are likely to maintain clinical response in Crohn's disease subjects. In addition, the PK/PD modeling results described below support the use of a 4-week dosing interval in Part II.

The high dose regimen for Part II is 400 mg at Week 0, 200 mg at Weeks 2 and 4, followed by 200 mg q4 weeks through Week 20. This dose regimen is predicted to result in a median anti-NKG2D antibody Cₘₐₓ in serum of 53.99 μg/mL, a median anti-NKG2D antibody serum concentration at Week 8 of 18.95 μg/mL, and a median trough serum anti-NKG2D antibody concentration at steady state of 12.00 μg/mL in subjects with Crohn's disease. Simulations results suggest that 89% of subjects on this high dose regimen are expected to maintain trough serum anti-NKG2D antibody concentrations >3 μg/mL at steady state, and the predicted median intestinal NKG2D RO is >96% (1).

The middle dose regimen for Part II is 150 mg SC at Week 0, 75 mg at Weeks 2 and 4, followed by 75 mg q4w through Week 20. This middle dose regimen is predicted to result in approximately 38% of the systemic exposure achieved with the high dose regimen. The predicted median intestinal NKG2D RO at steady state is 87%.

The low dose regimen for Part II is 50 mg SC at Week 0, 25 mg at Weeks 2 and 4, followed by 25 mg q4 weeks through Week 20. The low dose regimen is selected to explore the minimum effective dose of anti-NKG2D antibody in subjects with Crohn's disease. The predicted median serum trough concentration of anti-NKG2D antibody at steady state is 0.74 μg/mL which is predicted to result in a median NKG2D RO of 64% in the intestine. Furthermore, in the FIH study in RA subjects, a decrease in NKG2D expression on NK cells was not observed until the dose of anti-NKG2D antibody was mg/kg q2w, and decreases in NKG2D expression on both CD8+ T cells and NK cells were not observed until the dose was mg/kg q2w. These observations suggest that a dose regimen of at least 0.3 mg/kg q2w may be required to produce pharmacological effects, and the selected low maintenance dose regimen of 25 mg q4w would likely produce treatment effect at the lower part of the exposure-response curve.

### Part I and Part II

The selection of the 4 different dosing regimens of the anti-NKG2D antibody in Part I and Part II was based on all available PK, efficacy and safety data from the previous clinical studies and from a 52-week repeat-dose toxicology study. It should be noted that the current available information on the anti-NKG2D antibody has not established the relationship between the NKG2D RO and clinical effects of the drug. In addition, the currently reported antibody distribution coefficients for intestinal tissues may have limitations and the anti-NKG2D antibody concentrations in the intestine may not be accurately predicted. Nevertheless, the use of 4 different dose regimens of anti-NKG2D antibody in Part I and Part II, which will provide a wide range of drug exposures, is anticipated to provide a robust characterization of the exposure-response relationship of anti-NKG2D antibody in the treatment of Crohn's disease.

### Ustekinumab

Results from the 2 Phase 3 induction studies of intravenous (IV) ustekinumab and 1 Phase 3 maintenance study with SC ustekinumab were used to determine the appropriate dose regimen of ustekinumab for the treatment of Crohn's disease. As a result, a single IV induction dose of 6 mg/kg ustekinumab (administered as body weight-based tiered fixed doses) at Week 0, followed by SC maintenance therapy of 90 mg every 8 weeks has been demonstrated to provide robust efficacy across a range of endpoints including patient-reported outcomes, objective measures of inflammation, and health-related quality of life measures, as well as a favorable safety profile.

### Example 14

### Subject Population

Screening for eligible subjects will be performed within 5 weeks before administration of the study drug.

The inclusion and exclusion criteria for enrolling subjects in the 3 studies are described in the following 2 subsections. If there is a question about the inclusion or exclusion criteria, the investigator must consult with the appropriate sponsor representative and resolve any issues before enrolling a subject in the study. Waivers are not allowed.

The following inclusion and exclusion criteria apply to all three studies within this protocol.

### Inclusion Criteria

Each potential subject must satisfy all of the following criteria to be enrolled in the study:
1. Be a man or woman years of age.
2. Have Crohn's disease or fistulizing Crohn's disease of at least 3 months' duration, with colitis, ileitis, or ileocolitis, confirmed at any time in the past by radiography, histology, and/or endoscopy.
3. Have active Crohn's disease, defined as a baseline CDAI score of □220 but □450.
4. Have at least one of the following at screening:
   a. An abnormal CRP (>0.3 mg/dL [>3.0 mg/L]) OR
   b. Calprotectin >250 mg/kg.
5. In Part I, meet the following requirements for prior or current medications for Crohn's disease:
   a. Has previously demonstrated inadequate response, loss of response, or intolerance to 1 or more approved biologic therapies (eg, infliximab, adalimumab, certolizumab pegol, or vedolizumab) OR
   b. Has failed conventional therapy:

1) Is currently receiving corticosteroids and/or immunomodulators (i.e., AZA, 6-MP, or MTX) at adequate therapeutic doses. OR
2) Has a history of failure to respond to or tolerate an adequate course of corticosteroids and/or immunomodulators (i.e., AZA, 6-MP, or MTX). OR
3) Is corticosteroid dependent or has had a history of corticosteroid dependency.
6. In Part II, meet the following requirement for prior or current medications for Crohn's disease: has previously demonstrated inadequate response, loss of response, or intolerance to 1 or more approved biologic therapies (eg, infliximab, adalimumab, certolizumab pegol, or vedolizumab).
7. Adhere to the following requirements for concomitant medication for the treatment of Crohn's disease, which are permitted provided that doses meeting these requirements are stable, or have been discontinued, for at least 3 weeks before baseline (Week 0), unless otherwise specified:
   a. Oral 5-aminosalicylic acid (5-ASA) compounds.
   b. Oral corticosteroids at a prednisone-equivalent dose at or below 40 mg/day, or 9 mg/day of budesonide, or 5 mg/day beclomethasone dipropionate.
   c. Antibiotics being used as a primary treatment of Crohn's disease.
   d. Conventional immunomodulators (i.e., AZA, 6-MP, or MTX): subjects must have been taking them for at least 12 weeks and at a stable dose for at least 4 weeks before baseline.
8. A subject with a family history of colorectal cancer, personal history of increased colorectal cancer risk, age >50 years, or other known risk factor must be up-to-date on colorectal cancer surveillance (may be performed during screening). Adenomatous polyps must be removed before the first administration of study agent.
9. A subject who has had extensive colitis for ≥8 years, or disease limited to the left side of the colon for ≥12 years, must either have had a colonoscopy to assess for the presence of dysplasia within 1 year before the first administration of study agent or a colonoscopy to assess for the presence of malignancy at the screening visit, with no evidence of malignancy.
10. Have screening laboratory test results within the following parameters:
   a. Hemoglobin ≥8.0 g/dL.
   b. White blood cell count (WBCs) ≥3.0×10³/μL.
   c. Neutrophils ≥1.5×10³/μL.
   d. Platelets ≥100×10³/μL.
   e. Serum creatinine <1.7 mg/dL.
   f. Alanine aminotransferase (ALT) and aspartate aminotransferase (AST) concentrations must be within 2 times the upper limit of the normal range (ULN) range for the laboratory conducting the test.
   g. Direct (conjugated) bilirubin <1.0 mg/dL.
11. Are considered eligible according to the following tuberculosis (TB) screening criteria:
   a. Have no history of latent or active TB before screening. Exceptions are made for subjects currently receiving treatment for latent TB, if there is no evidence of active TB, or who have a history of latent TB and documentation of having completed adequate treatment for latent TB within 3 years before the first administration of study agent. It is the responsibility of the investigator to verify the adequacy of previous TB treatment and provide appropriate documentation. Note: The exceptions outlined above exclude subjects in countries with high multidrug-resistant TB burden (eg, Brazil, China, India, the Russian Federation, and South Africa), due to potential concerns for multi-drug-resistant TB.
   b. Have no signs or symptoms suggestive of active TB upon medical history and/or physical examination.
   c. Have had no recent close contact with a person with active TB or, if there has been such contact, will be referred to a physician specializing in TB to undergo additional evaluation and, if warranted, receive appropriate treatment for latent TB before or simultaneously with the first administration of study agent.
   d. Criterion modified per Amendment 1.
      d.1 Within 2 months before the first administration of study agent, either have negative QuantiFERON-TB Gold test, or have a newly identified positive QuantiFERON-TB Gold test in which active TB has been ruled out, and for which appropriate treatment for latent TB has been initiated either before or simultaneously with the first administration of study agent (except in countries with high multidrug-resistant TB burden [eg, Brazil, China, India, the Russian Federation, and South Africa]), where subjects with a newly identified positive QuantiFERON-TB Gold test result are excluded). Indeterminate results should be handled as outlined. A negative tuberculin skin test is additionally required if the QuantiFERON-TB gold test is not approved/registered in that country. A tuberculin skin test is recommended but not required for study centers in countries where tuberculin is not available. The QuantiFERON-TB Gold In-Tube test is not required at screening for subjects with a history of latent TB and appropriate treatment as described above in Inclusion Criterion.
   e. Have a chest radiograph (posterior-anterior and lateral views), taken within 3 months before the first administration of study agent and read by a qualified radiologist, with no evidence of current active TB or old inactive TB.
12. A woman of childbearing potential must have a negative highly sensitive serum β-human chorionic gonadotropin [β-hCG]) pregnancy test result at screening and a negative urine pregnancy test result at Week 0.
13. Before randomization, a female subject must be either:
   a. Not of childbearing potential, defined as:
      1) Premenarchal: A premenarchal state is one in which menarche has not yet occurred.
      2) Postmenopausal: A postmenopausal state is defined as no menses for 12 months without an alternative medical cause. A high follicle-stimulating hormone (FSH) level (>40 IU/L or mIU/mL) in the postmenopausal range may be used to confirm a postmenopausal state in women not using hormonal contraception or hormonal replacement therapy; however, in the absence of 12 months of amenorrhea, a single FSH measurement is insufficient.
      3) Permanently sterile: Permanent sterilization methods include hysterectomy, bilateral salpingectomy, bilateral tubal occlusion/ligation procedures, and bilateral oophorectomy. OR
   b. Of childbearing potential and:
      1) Practicing a highly effective method of contraception (failure rate of <1% per year when used consistently and correctly), consistent with local regulations regarding the use of contraceptive methods for subjects participating in clinical studies. Examples of highly effective contraceptives include user-independent methods such as implantable progestogen-only hormone contraception associated with inhibition of ovulation; intrauterine device (IUD); intrauterine hormone-releasing system (IUS); vasectomized partner; or sexual abstinence (considered a highly effective method only if defined as refraining from heterosexual intercourse during the entire period of risk associated with the study drug, and if in line with the preferred and usual lifestyle of the subject); or user-dependent methods such as combined (estrogen- and progestogen-containing) hormonal contraception associated with inhibition of ovulation (oral, intravaginal, transdermal); or progestogen-only hormone contraception associated with inhibition of ovulation (oral, injectable).
      2) Agrees to remain on a highly effective method of contraception throughout the study and for at least 12 weeks (16 weeks for subjects in Part II who discontinue study agent before or at Week 20) after the last administration of study agent.
   Note: If a subject's childbearing potential changes after start of the study (e.g., a premenarchal woman experiences menarche) or the risk of pregnancy changes (e.g., a woman who is not heterosexually active becomes active), a woman must begin a highly effective method of contraception, as described throughout the inclusion criteria.
14. A woman must agree not to donate eggs (ova, oocytes) for the purposes of assisted reproduction during the study and for a period of 12 weeks (16 weeks for subjects in Part II who discontinue study agent before or at Week 20) after the last administration of study agent.
15. During the study and for at least 12 weeks (16 weeks for subjects in Part II who discontinue study agent before or at Week 20) after the last administration of study agent, a man
   a. who is sexually active with a woman of childbearing potential must agree to use a barrier method of contraception (e.g., condom with spermicidal foam/gel/film/cream/suppository).
   b. who is sexually active with a pregnant woman must use a condom.
   c. must agree not to donate sperm.
16. Be willing and able to adhere to the prohibitions and restrictions specified in this protocol.
17. Must sign an informed consent form (ICF) indicating that he or she understands the purpose of and procedures required for the study and is willing to participate in the study.
18. Criterion modified per Amendment 1.
   18.1 DNA sample collection for SNP testing is required for all subjects in this study. Each subject must have a SNP status of either positive or negative. Each subject must sign a separate ICF if he or she agrees to consent to additional optional DNA research where local regulations permit. Refusal to give consent for the optional DNA research does not exclude a subject from participation in the study.

### Exclusion Criteria

Any potential subject who meets any of the following criteria will be excluded from participating in the study:
1. Has complications of Crohn's disease such as symptomatic strictures or stenoses, short gut syndrome, or any other manifestation that might be anticipated to require surgery, could preclude the use of the CDAI to assess response to therapy, or would possibly confound the ability to assess the effect of treatment with the anti-NKG2D antibody or ustekinumab.
2. Currently has or is suspected to have an abscess. Recent cutaneous and perianal abscesses are not exclusionary if drained and adequately treated at least 3 weeks before baseline, or 8 weeks before baseline for intra-abdominal abscesses, provided that there is no anticipated need for any further surgery. Subjects with active fistulas may be included if there is no anticipation of a need for surgery and there are currently no abscesses identified.
3. Has had any kind of bowel resection within 6 months or any other intra-abdominal surgery within 3 months before baseline.
4. Has a draining (i.e., functioning) stoma or ostomy.
5. Has received any of the following prescribed medications or therapies within the specified period:
   a. IV corticosteroids <3 weeks before baseline.
   b. Other oral immunomodulatory agents (e.g., 6-thioguanine [6-TG], cyclosporine, tacrolimus, sirolimus, or mycophenolate mofetil, tofacitinib and other Janus kinase [JAK] inhibitors) <6 weeks or within 5 half-lives of agent before baseline, whichever is longer.
   c. Nonbiologic experimental or investigational agents <4 weeks or within 5 half-lives of agent before baseline, whichever is longer.
   d. Nonautologous stem cell therapy (e.g., Prochymal), natalizumab, efalizumab, or biologic agents that deplete B or T cells (e.g., rituximab, alemtuzumab, or visilizumab) <12 months before baseline.
   e. TNFα-antagonist biologic agents (e.g., mAb therapies) or other agents intended to suppress or eliminate TNFα <8 weeks before baseline.
   f Vedolizumab <16 weeks before baseline.
   g. Other immunomodulatory biologic agents <12 weeks or within 5 half-lives of agent before baseline, whichever is longer.
   h. Treatment with apheresis (e.g., Adacolumn apheresis) or total parenteral nutrition as a treatment for Crohn's disease <3 weeks before baseline.
6. Has a stool culture or other examination positive for an enteric pathogen, including Clostridium difficile toxin, in the last 4 months unless a repeat examination is negative and there are no signs of ongoing infection with that pathogen.
7. Has previously received a biologic agent targeting IL-12 or IL-23, including but not limited to ustekinumab or briakinumab (ABT-874).
8. Has previously received the anti-NKG2D antibody.
9. Has received a Bacille Calmette-Gurin (BCG) vaccination within 12 months or any other live bacterial or live viral vaccination within 12 weeks before baseline.
10. Has a history of, or ongoing, chronic or recurrent infectious disease, including but not limited to, chronic renal infection, chronic chest infection, recurrent urinary tract infection (e.g., recurrent pyelonephritis or chronic nonremitting cystitis), or open, draining, or infected skin wounds or ulcers.
11. Has current signs or symptoms of infection. Established nonserious infections (e.g., acute upper respiratory tract infection, simple urinary tract infection) need not be considered exclusionary at the discretion of the investigator.
12. Has a history of serious infection (e.g., sepsis, pneumonia, or pyelonephritis), including any infection requiring hospitalization or IV antibiotics, for 8 weeks before baseline.
13. Has evidence of a Herpes zoster infection ≤8 weeks before baseline.
14. Has a history of latent or active granulomatous infection, including histoplasmosis or coccidioidomycosis, before screening. Refer to Inclusion Criteria 11 a for information regarding eligibility with a history of latent TB.
15. Has evidence of current active infection, including TB, or a nodule suspicious for lung malignancy on screening or any other available chest radiograph, unless definitively resolved surgically or by additional imaging and with source document confirmation.
16. Has or ever has had a nontuberculous mycobacterial infection or serious opportunistic infection (e.g., cytomegalovirus colitis, Pneumocystis carinii, aspergillosis).
17. Has a history of human immunodeficiency virus (HIV) antibody positivity, or tests positive for HIV at screening.
18. Are seropositive for antibodies to hepatitis C virus (HCV) without a history of successful treatment, defined as being negative for HCV RNA at least 24 weeks after completing antiviral treatment.
19. Subjects must undergo screening for hepatitis B virus (HBV). At a minimum, this includes testing for HBV surface antigen (HBsAg), HBV surface antibody (anti-HBs), and HBV core antibody (anti-HBc) total:
   a. Subjects who test negative for all HBV screening tests (i.e., HBsAg-, anti-HBc-, and anti-HBs-) are eligible for this study.
   b. Subjects who test positive for surface antigen (HBsAg+) are not eligible for this study, regardless of the results of other hepatitis B tests.
   c. Subjects who test negative for surface antigen (HBsAg-) and test positive for core antibody (anti-HBc+) and surface antibody (anti-HBs+) are eligible for this study.
   d. Subjects who test positive only for surface antibody (anti-HBs+) are eligible for this study.
   e. Subjects who test positive only for core antibody (anti-HBc+) must undergo further testing for hepatitis B DNA acid (HBV DNA test). If the HBV DNA test is positive, the subject is not eligible for this study. If the HBV DNA test is negative, the subject is eligible for this study. In the event the HBV DNA test cannot be performed, the subject is not eligible for this study.
   Note: For subjects who are not eligible for this study due to HIV, HCV, and HBV test results, consultation with a physician with expertise in the treatment of those infections is recommended.
20. Has severe, progressive, or uncontrolled renal, hepatic, hematological, endocrine, pulmonary, cardiac, neurologic, cerebral, or psychiatric disease, or signs and symptoms thereof.
21. Has a transplanted organ (with exception of a corneal transplant >12 weeks before screening).
22. Has a known history of lymphoproliferative disease, including monoclonal gammopathy of unknown significance (MGUS), lymphoma, or signs and symptoms suggestive of possible lymphoproliferative disease, such as lymphadenopathy and/or splenomegaly.
23. Has any known malignancy or has a history of malignancy (with the exception of basal cell carcinoma; squamous cell carcinoma in situ of the skin; or cervical carcinoma in situ that has been treated with no evidence of recurrence; or squamous cell carcinoma of the skin that has been treated with no evidence of recurrence within 5 years before screening).
24. Is unable or unwilling to undergo multiple venipunctures because of poor tolerability or lack of easy access to veins.
25. Is known to have had a substance abuse (drug or alcohol) problem within the previous 12 months before baseline.
26. Has known allergies, hypersensitivity, or intolerance to the anti-NKG2D antibody or ustekinumab or any of their excipients (refer to IBs).
27. Are currently or intending to participate in any other study using an investigational agent or procedure during participation in this study.
28. Is a woman who is pregnant, or breast-feeding, or planning to become pregnant or is a man who plans to father a child while enrolled in this study or within 12 weeks (16 weeks for subjects in Part II who discontinue study agent before or at Week 20) after the last administration of study agent.
29. Has any condition that, in the opinion of the investigator, would make participation not be in the best interest (e.g., compromise the well-being) of the subject or that could prevent, limit, or confound the protocol-specified assessments.
30. Is an employee of the investigator or study site, with direct involvement in the proposed study or other studies under the direction of that investigator or study site, as well as family members of the employees or the investigator.

### Example 15

### Treatment Allocation and Blinding

### Treatment Allocation

Central randomization will be implemented in this study. Subjects will be randomly assigned to 1 of 2 treatment groups (1:1 ratio) in Part I and to 1 of 5 treatment groups (1:1:1:1:1 ratio) in Part II, based on a computer-generated randomization schedule prepared before the study by or under the supervision of the sponsor. Each of the 3 studies will have separate randomizations. Each randomization will be balanced by using randomly permuted blocks and will be stratified by baseline CDAI score (≤300 or >300) and SNP-positive status (yes or no). The interactive web response system (IWRS) will assign a unique treatment code, which will dictate the treatment assignment and matching study drug kit for the subject. The requestor must use his or her own user identification and personal identification number when contacting the IWRS, and will then give the relevant subject details to uniquely identify the subject.

### Blinding

To maintain the study blind, the study agent container will have a label containing the study name and medication number or syringe number. The label will not identify the study agent in the container. The medication number or syringe number will be entered in the case report form (CRF) when the drug is dispensed. The study agents will be identical in appearance and packaging.

Planned efficacy and safety evaluations will be performed after the following planned DBLs (additional DBLs may occur and would be described in the statistical analysis plan):
Interim analysis lock (Study 1: Bio-IR subjects): Occurs when approximately 80% of the Part 1 Bio-IR subjects (at least 40 Bio-IR subjects and at least 30 Bio-IR/SNP-positive subjects per treatment group) have completed their Week 8 visit or have terminated their study participation before Week 8.
Week 12 DBL for Part I Bio-IR (Study 1): Occurs when all Part I Bio-IR subjects have completed their Week 12 visit or have terminated their study participation before Week 12.
Week 12 DBL for Part I Bio-NF (Study 2; optional): This DBL would occur if the decision is made not to initiate Part II based on the Week 12 DBL for Part I Bio-IR subjects; a dedicated DBL would occur when all Bio-NF subjects have completed their Week 12 visit or have terminated their study participation before Week 12.
Week 12 DBL for Part I Bio-NF (Study 2) and Part II (Study 3): Occurs when all Part I Bio-NF and Part II subjects have completed their Week 12 visit or have terminated their study participation before Week 12.
Week 24 DBL (Studies 1, 2, and 3): Occurs when all Part I and Part II subjects have completed their Week 24 visit or have terminated their study participation before Week 24.
Final DBL (Studies 1, 2, and 3): Occurs when all Part I and Part II subjects have completed their final efficacy and safety visit or have terminated their study participation before the final efficacy and safety visit.

At the time of DBLs that occur before the Week 12 DBL for Part I Bio-NF and Part II, a limited number of sponsor personnel will become unblinded to treatment assignment. At the time of the Week 12 DBL for Part I Bio-NF and Part II, the sponsor, except for site monitors (who have interactions with the investigative sites), will become unblinded to treatment assignment. Identification of sponsor personnel who will have access to subject-level data before the Week 12 DBL for Part I Bio-NF and Part II will be documented before the unblinding. The study blind will be maintained for investigators, site personnel, subjects, and sponsor site monitors until the final analyses have been completed for all subjects in the study. This measure will mitigate the potential bias in the remaining investigator and subject assessments.

Data that may potentially unblind the treatment assignment (i.e., study drug serum concentrations, anti-drug antibodies, treatment allocation, and study drug preparation/accountability data) will be handled with special care to ensure that the integrity of the blind is maintained and the potential for bias is minimized. In particular, before unblinding, this information will be available only to a limited number of data management staff for purposes of data cleaning, and if applicable, to quality assurance representatives for the purposes of conducting independent drug audits.

The SNP status and postbaseline results of CRP, fecal lactoferrin, and fecal calprotectin tests will be blinded to the investigative site. If an investigative site requests these data, it will be provided to them after the final analyses have been completed.

The designated pharmacists, or other appropriately licensed and authorized personnel, and independent drug monitors will be unblinded to study agent. Placebo infusions/injections will have the same appearance as the ustekinumab infusions/anti-NKG2D antibody injections. Under no circumstances should unblinded personnel reveal the treatment assignment for a subject.

For bioanalytical purposes, before the PK, anti-drug antibody, and PD bioanalyses are initiated, the unblinded data management team will provide the sponsor bioanalysts with the information about which treatment (anti-NKG2D antibody, ustekinumab, or placebo) the subjects received, but not the dose level to which the subjects are randomized. For the purpose of performing PK, immunogenicity, and PD bioanalyses, bioanalysts in Biologics Clinical Pharmacology at Janssen will be unblinded to treatment-level data (anti-NKG2D antibody, ustekinumab, or placebo) at the time of analyzing serum samples for the determination of drug concentrations, detection of antibodies to study agents, or PD assessments. Samples will be separated based on treatment administered; subject identification and dose given will not be disclosed.

Additionally, a given subject's treatment assignment may be unblinded to the sponsor, Institutional Review Board/Independent Ethics Committee (IRB/IEC), and site personnel to fulfill regulatory reporting requirements for suspected unexpected serious adverse reactions (SUSARs).

The investigator will not be provided with randomization codes. The codes will be maintained within the IWRS, which has the functionality to allow the investigator to break the blind for an individual subject.

Under normal circumstances, the blind should not be broken until the final analyses have been completed for all subjects. Otherwise, the blind should be broken only if specific emergency treatment/course of action would be dictated by knowing the treatment status of the subject. In such cases, the investigator may in an emergency determine the identity of the treatment by contacting the IWRS. It is recommended that the investigator contact the sponsor or its designee if possible to discuss the particular situation, before breaking the blind. Telephone contact with the sponsor or its designee will be available 24 hours per day, 7 days per week. In the event the blind is broken, the sponsor must be informed as soon as possible. The date, time, and reason for the unblinding must be documented by the IWRS, in the appropriate section of the CRF, and in the source document. The documentation received from the IWRS indicating the code break must be retained with the subject's source documents in a secure manner so as to not unblind the study site monitor. The investigators are advised not to reveal the study treatment assignment to the study site monitor or to sponsor personnel.

A separate code-break procedure will be available for use by the Janssen Global Medical Safety group to allow for unblinding of individual subjects to comply with specific requests from regulatory or health authorities.

Subjects who have had their treatment assignment unblinded will be discontinued from further study agent administration but should continue to return for scheduled evaluations (Section Error! Reference source not found.).

### Example 16

### Dosage and Administration

### Part I

In Part I of the study (FIG. 2), all subjects will receive either placebo SC or anti-NKG2D antibody 400 mg SC at Week 0 and placebo SC or anti-NKG2D antibody 200 mg SC at Weeks 2, 4, 6, 8, 10, 12, 14, 16, 18, 20, and 22, with the exception of placebo nonresponders at Week 12. Placebo nonresponders will receive anti-NKG2D antibody 400 mg SC at Week 12 and anti-NKG2D antibody 200 mg SC at Weeks 14, 16, 18, 20, and 22. Study drug concentration in Part I is 100 mg/mL.

To maintain the blind in Part I, all subjects will receive 4 SC injections at Weeks 0 and 12, and 2 SC injections at Weeks 2, 4, 6, 8, 10, 14, 16, 18, 20, and 22.

### Part II

In Part II of the study (FIG. 3), subjects will be randomly assigned in equal proportions to receive placebo, 1 of 3 dose regimens of anti-NKG2D antibody, or ustekinumab, as follows:
Placebo: Placebo SC at Weeks 0, 2, 4, 8, 12, 16, and 20. Placebo nonresponders at Week 12 will receive anti-NKG2D antibody 150 mg SC at Week 12 and anti-NKG2D antibody 75 mg SC at Weeks 14, 16, and 20.
Anti-NKG2D antibody high dose: 400 mg SC at Week 0 and 200 mg SC at Weeks 2, 4, 8, 12, 16, and 20. (Study drug concentration=100 mg/mL)
Anti-NKG2D antibody middle dose: 150 mg SC at Week 0 and 75 mg SC at Weeks 2, 4, 8, 12, 16, and 20. (Study drug concentration=100 mg/mL and 50 mg/mL at Week 0, 75 mg/mL for subsequent doses)
Anti-NKG2D antibody low dose: 50 mg SC at Week 0 and 25 mg SC at Weeks 2, 4, 8, 12, 16, and 20. (Study drug concentration=50 mg/mL at Week 0, 25 mg/mL for subsequent doses)
Ustekinumab: tiered doses approximating 6 mg/kg IV (Section 0) at Week 0 and 90 mg SC at Weeks 8 and 16.

Administration of IV study agent at Week 0 should occur over a period of not less than 1 hour. The infusion should be completed within 5 hours of preparation.

To maintain the blind in Part II, all subjects will receive four SC injections plus an IV infusion at Week 0, two SC injections at Weeks 2, 4, 12, and 20, three SC injections at Weeks 8 and 16, and one SC injection at Week 14.

### Treatment Compliance

Study agent will be administered as an IV infusion or SC injection by qualified staff. The details of each administration will be recorded in the CRF. For IV infusions, this will include date and start and stop times of the IV infusion and volume infused; for SC injections, this will include date and time of SC injection.

### Example 17

### Efficacy Evaluations

The CDAI will be the primary tool for assessing disease activity response to the anti-NKG2D antibody, along with PRO-2, PRO-3, Bristol stool form scale, and abdominal pain based on NRS 0-10 scale. The degree of inflammation will be assessed by measuring serum CRP concentrations. Stool samples will be collected and analyzed to evaluate changes in markers that may reflect the anti-NKG2D antibody or ustekinumab treatment. The well-being of subjects will be measured using the IBDQ and the SF-36. Mucosal healing will be assessed by ileocolonoscopy. For subjects with fistulizing disease, fistula closure will also be assessed.

### Crohn's Disease Activity Index

The CDAI will be assessed by collecting information on 8 different Crohn's disease-related variables extra-intestinal manifestations, abdominal mass, weight, hematocrit, total number of liquid stools, abdominal pain/cramping, use of antidiarrheal drug(s) and/or opiates, and general well-being. The last 4 variables are scored over 7 days by the subject on a diary card. The PRO-2 score is based on the CDAI components of the total number of liquid stools and abdominal pain/cramping. The PRO-3 score, which is also based on the CDAI, comprises the PRO-2 components plus general well-being. Subjects are to complete a daily diary entry and bring the diary to each visit.

### Bristol Stool Form Scale

The Bristol stool form scale is a medical aid to classify the form (or consistency) of human feces into 7 categories. It has been used as a research tool to evaluate the effectiveness of treatments for various diseases of the bowel (e.g., irritable bowel syndrome). Subjects will complete the Bristol stool form scale as a daily diary entry and bring the diary to each visit up to Week 12.

### Abdominal Pain Numerical Rating Scale

The NRS for pain is a unidimensional measure of pain intensity in adults. An 11-point (0-10) NRS will be used to evaluate abdominal pain. The score of 0 represents “no pain” and the score of 10 represents the “pain as bad as you can imagine”, with greater scores indicating greater pain severity and intensity. Subjects will select only one number that best reflects their pain at its worst in the past 24 hours. The abdominal pain NRS will be assessed daily, Subjects are to complete a daily diary entry and bring the diary to each visit

### C-Reactive Protein

C-reactive protein has been demonstrated to be useful as a marker of inflammation in patients with inflammatory bowel disease (IBD). In Crohn's disease, elevated CRP concentrations have been associated with severe clinical activity, elevated sedimentation rate, and active disease as detected by colonoscopy. Error! Reference source not found.,Error! Reference source not found. Blood samples for the measurement of CRP will be collected from all subjects at visits indicated in the Time and Events Schedule. CRP will be assayed using a validated, high sensitivity CRP assay. Results of postbaseline CRP measurement will not be released to the investigators by the central laboratory.

### Fecal Lactoferrin and Calprotectin

Fecal lactoferrin and fecal calprotectin have been demonstrated to be sensitive and specific markers in identifying intestinal inflammation and response to treatment in patients with IBD. Stool samples for fecal lactoferrin and calprotectin concentrations will be collected from all subjects at visits indicated in the Time and Events Schedules. Assays for fecal lactoferrin and calprotectin concentrations will be performed using a validated method. Additional tests may also be performed on the stool samples for additional markers related to intestinal inflammation and treatment response such as the microbiome. Results of postbaseline fecal lactoferrin and calprotectin tests will not be released to the investigators by the central laboratory.

### Inflammatory Bowel Disease Questionnaire

The IBDQ is a 32-item self-report questionnaire for subjects with IBD to evaluate the PROs across 4 dimensions: bowel symptoms (loose stools, abdominal pain), systemic symptoms (fatigue, altered sleep pattern), social function (work attendance, need to cancel social events), and emotional function (anger, depression, irritability). Scores range from 32 to 224 with higher scores indicating better outcomes.

### 36-Item Short-Form Health Survey

The SF-36 was developed to measure the general health status with 8 functional domain scales.

Limitations in physical functioning due to health problems.

Limitations in usual role activities due to physical health problems.

Bodily pain.

General mental health (psychological distress and well-being).

Limitations in usual role activities due to personal or emotional problems.

Limitations in social functioning due to physical or mental health problems.

Vitality (energy and fatigue).

General health perception.

Based on the 8 scale scores, the Physical Component Summary (PCS) and the Mental Component Summary (MCS) can be derived. The scale scores and summary scores are converted into a score from 0 to 100 using a norm-based system where linear transformations are performed to transform scores to a mean of 50 and standard deviations of 10, based on general US population norms. The concepts measured by the SF-36 are not specific to any age, disease, or treatment group, allowing comparison of relative burden of different diseases and the relative benefit of different treatments.

### Fistula Assessment

All subjects will be assessed for fistulas. For subjects with fistulizing disease, fistula closure will be assessed. Enterocutaneous fistulas (e.g., perianal and abdominal) will be considered no longer draining (i.e., closed) when there is absence of drainage despite gentle compression. Rectovaginal fistulas will be considered closed based on either physical examination or absence of relevant symptoms (e.g., passage of rectal material or flatus from the vagina).

### Endoscopic Endpoints

Mucosal healing will be assessed during endoscopy (ileocolonoscopy). A video ileocolonoscopic examination will be performed to determine the presence or absence of mucosal inflammation and ulceration at screening, Week 12, and Week 24, according to the study reference manual provided to each site; if the video ileocolonoscopic examination is not performed on the day of the visit, it must be performed at least 8 days before the Week 0 visit and no more than 8 days before the Week 12 visit. The Week 24 video ileocolonoscopy is suggested but not required; if performed, it should occur not more than 8 days before the Week 24 visit. Video endoscopies will be assessed by a central facility that will be blinded to treatment group and study visit. A complete video endoscopic examination does not require assessment of the terminal ileum if it cannot be visualized.

The SES-CD score is based on the evaluation of 4 endoscopic components (presence/size of ulcers, proportion of mucosal surface covered by ulcers, proportion of mucosal surface affected by any other lesions, and presence/type of narrowing/strictures) across 5 ileocolonic segments. Each endoscopic component is scored from 0 to 3 for each segment, and a total score is derived from the sum of all the component scores (range, 0 to 56). The SES-CD score will be evaluated by a central reader.

In addition to the evaluation of the SES-CD score, endoscopic healing, which is traditionally defined as the resolution (absence) of mucosal ulcers in response to a therapeutic intervention, will also be assessed by the central reader.

### Example 18

### Pharmacokinetics and Immunogenicity Evaluations

### Evaluations

Serum samples will be used to evaluate the PK and immunogenicity of the anti-NKG2D antibody and ustekinumab (antibodies to the anti-NKG2D antibody and antibodies to ustekinumab). Samples collected for analyses of serum concentration of anti-NKG2D antibody and ustekinumab and antibodies to the anti-NKG2D antibody or ustekinumab may additionally be used to evaluate safety or efficacy aspects that address concerns arising during or after the study period, for further characterization of immunogenicity or for the evaluation of relevant biomarkers. Genetic analyses will not be performed on these serum samples. Subject confidentiality will be maintained.

At visits where only serum concentration of study agent will be evaluated (i.e., no antibodies to study agent will be evaluated), 1 venous blood sample of sufficient volume should be collected, and each serum sample should be divided into 2 aliquots (1 for serum concentration of study agent, and a back-up). At visits where serum concentration of study agent and antibodies to study agent will be evaluated, 1 venous blood sample of sufficient volume should be collected. Each serum sample will be divided into 3 aliquots (1 each for serum concentration of study agent, antibodies to study agent, and a back-up).

### Serum Concentration

Serum samples will be analyzed to determine concentrations of the anti-NKG2D antibody and ustekinumab using a validated, specific, and sensitive method by or under the supervision of the sponsor.

### Immunogenicity Assessments (Antibodies to Study Agent)

The detection and characterization of antibodies to the anti-NKG2D antibody and ustekinumab will be performed using a validated assay method by or under the supervision of the sponsor. All samples collected for detection of antibodies to the anti-NKG2D antibody or ustekinumab will also be evaluated for the anti-NKG2D antibody or ustekinumab serum concentration to enable interpretation of the antibody data.

Serum samples will be screened for antibodies binding to the anti-NKG2D antibody or ustekinumab and the titer of confirmed positive samples will be reported. Other analyses may be performed to verify the stability of antibodies to the anti-NKG2D antibody or ustekinumab and/or further characterize the immunogenicity of the anti-NKG2D antibody or ustekinumab. Antibodies to the anti-NKG2D antibody or ustekinumab will be evaluated on blood drawn from all subjects according to the Time and Events Schedule. Additionally, samples should also be collected at the final visit for subjects who terminate from the study. These samples will be tested by the sponsor or sponsor's designee.

### Example 19

### Biomarker and Other Pharmacodynamic Evaluations

Biomarker assessments will be made to examine the biological response to treatment and to identify biomarkers that are relevant to the anti-NKG2D antibody treatment and/or Crohn's disease. Assessments will include the evaluation of relevant biomarkers in serum, whole blood, stool, and mucosal biopsy samples collected according to the Time and Events Schedule.

### Serum-Based Biomarkers

Blood samples for serum-based biomarker analyses will be collected from all subjects. Assays to be performed may include the following: measurement of proteins associated with the NKG2D pathway, including but not limited to, MICA, MICB, and UBPs 1-6, as well as proteins associated with Crohn's disease such as SAA (serum amyloid A), IFNγ, or matrix metalloproteinases.

### Whole Blood-Based Biomarkers

Whole blood samples will be collected from all subjects to study the effect of study agent on RNA expression. Whole blood analyses may also examine RNA expression associated with the pathogenesis of Crohn's disease. An additional blood sample will be obtained for analysis of the TCR repertoire.

### Biopsy-Based Biomarkers

Mucosal biopsy samples will be collected during video ileocolonoscopy to study the effect of study agent on gene and protein expression and for the histologic assessment of disease and healing (refer to Study Reference Manual for further details). Mucosal biopsy analyses may also examine gene and protein expression associated with the pathogenesis of Crohn's disease.

### NKG2D Receptor Occupancy

NKG2D RO assessments will be performed at the time points specified in the Time and Events schedule. NKG2D RO will be determined using a validated flow cytometry assay.

### Immunophenotyping

Immunophenotyping assessments (including NK cells and CD8+ T cells) will be performed at the time points specified in the Time and Events schedule. Immunophenotyping will be performed using flow cytometry.

### Pharmacogenomic (DNA) Evaluations

All subjects will be tested for the NKG2D SNP rs2255336 and the MICB (NKG2D ligand) SNP rs2239705 at screening. For subjects who have signed a separate ICF, complete genomic testing will be done to search for links of specific genes to disease or response to drug. Only DNA research related to the anti-NKG2D antibody or ustekinumab or to the diseases for which this drug is developed will be performed. A 10 mL blood sample will be collected from all subjects for this testing; in the event of DNA extraction failure, a replacement pharmacogenomic blood sample will be requested from the subject. Further, a subject may withdraw his/her optional DNA consent for complete genomic testing at any time without affecting their participation in other aspects of the study, or their future participation in the study.

### Example 20

### Statistical Methods

Statistical analysis will be done by the sponsor or under the authority of the sponsor. A general description of the statistical methods to be used to analyze the efficacy and safety data is outlined below. Specific details will be provided in the Statistical Analysis Plan. Descriptive statistics (e.g., mean, median, standard deviation, interquartile range, minimum, and maximum) will be used to summarize continuous variables. Counts and percentages will be used to summarize categorical variables. Graphic data displays (e.g., line plots) may also be used to summarize the data.

Analyses suitable for categorical data (e.g., chi-square tests or Cochran-Mantel-Haenszel chi-square tests as appropriate) will be used to compare the proportions of subjects achieving selected endpoints (e.g., clinical remission). In cases of rare events, Fisher's exact test will be used for treatment comparisons. Continuous response parameters will be compared using an analysis of variance (ANOVA) or covariance (ANCOVA) model on the van der Waerden normal scores.

All statistical testing will be performed at a significance level of 0.05 (2-sided) unless otherwise specified. Nominal p-values will be displayed for all treatment comparisons.

### Example 21

### Sample Size Determination

Sample size calculations for all three studies (Study 1 [Part I Bio-IR subjects], Study 2 [Part I Bio-NF subjects], and Study 3 [Part II Bio-IR subjects]) were determined by the power to detect a significant difference in the change from baseline in the CDAI score at Week 8 (primary endpoint in each study) between the anti-NKG2D antibody and placebo using a 2-sample t-test.

### Sample Size in Part I

### Bio-IR Subjects (Study 1)

### All Bio-IR Subjects

The assumptions for the sample size calculations in Bio-IR subjects were based on data from CNTO1275CRD3002, a study conducted by the sponsor in subjects with Crohn's disease who had failed or were intolerant to TNF-antagonist therapy. In said study, the mean CDAI change from baseline at Week 8 was −25.1 (SD=91.41) and −78.7 (SD=91.79) and the proportion of subjects in clinical remission at Week 8 was 7% and 21% for the placebo and ustekinumab 6 mg/kg groups, respectively. These assumptions incorporated the impact of 6% of subjects being noncompleters.

For the current study, assuming the mean CDAI change from baseline at Week 8 is −79 in the anti-NKG2D antibody group and −25 in the placebo group with a common SD of 92, 50 subjects per treatment group will provide approximately 80% power to detect a treatment difference between the anti-NKG2D antibody and placebo at an overall Type 1 error of 0.05 (2-sided; Table).

The power calculations for clinical remission are based on the potential to demonstrate 10% greater efficacy for the anti-NKG2D antibody than was previously observed for ustekinumab. Fifty Bio-IR subjects per treatment group in Study 1 will also provide 90% power to detect a difference from placebo in the proportion of subjects in clinical remission at Week 8 at an overall Type 1 error of 0.05 (2-sided; Table), assuming the anti-NKG2D antibody has a remission rate of 31%, which is 10% greater than the ustekinumab remission rate in the previous study.

### Example 22

### Bio-IR Subjects Who Are SNP-Positive (Bio-IR/SNP+)

As described earlier, a post hoc analysis of efficacy data in the prior Phase 2a study demonstrated greater efficacy in a subgroup of subjects who were SNP-positive. Therefore, the association between SNP-positive status and higher clinical efficacy is being prospectively examined in this study. Based on the assumption that 75% of the Crohn's disease population will be SNP-positive, 50 Bio-IR subjects will provide approximately 38 Bio-IR/SNP+ subjects. Thirty-eight Bio-IR/SNP+ subjects per group will provide 80% power to detect a difference from placebo in the proportion of subjects in clinical remission at Week 8 at an overall Type 1 error of 0.05 (2-sided; Table 3), assuming the anti-NKG2D antibody has a remission rate of 31%, which is 10% greater than the ustekinumab remission rate in that study.

TABLE 3
Power to detect a treatment difference and sample size combinations
at an overall Type 1 error of 0.05 (2-sided)

| | | | | |
|---|---|---|---|---|
| | | anti- | | |
| Sample size | | NKG2D | | |
| per group | Placebo | antibody | Difference | Power |

Mean change from baseline in CDAI
score at Week 8
Based on assumptions from
study CNTO1275CRD3001

| | | | | |
|---|---|---|---|---|
| 50 | −25 | −79 | 54 | 83%* |

CDAI score for anti-NKG2D antibody is derived
based on 10% greater remission rate for
anti-NKG2D antibody than ustekinumab
in study CNTO1275CRD3002

| | | | | |
|---|---|---|---|---|
| 50 | −66 | −152 | 86 | 99%** |

Clinical remission at Week 8
Based on 10% greater remission rate for
anti-NKG2D antibody than ustekinumab
in study CNTO1275CRD3001

| | | | | |
|---|---|---|---|---|
| 38 | 7% | 31% | 24% | 80% |
| 50 | 7% | 31% | 24% | 90% |

Based on assumptions from
study CNTO1275CRD3001

| | | | | |
|---|---|---|---|---|
| 50 | 7% | 21% | 14% | 56% |

Based on 10% greater remission rate for
anti-NKG2D antibody than ustekinumab
in study CNTO1275CRD3002

| | | | | |
|---|---|---|---|---|
| 38 | 20% | 50% | 30% | 80% |
| 50 | 20% | 50% | 30% | 89% |

*Assuming a standard deviation of 92 for each group.
**Assuming a standard deviation of 100 for each group.

### Example 23

### Bio-NF Subjects (Study 2)

### All Bio-NF Subjects

The assumptions for the sample size calculations in Bio-NF subjects were based on data from CNTO1275CRD3002, a study conducted by the sponsor in subjects with Crohn's disease who had failed or were intolerant to corticosteroids or immunomodulators but who had not failed TNF-antagonist therapy. In CNTO1275CRD3002, the mean CDAI change from baseline at Week 8 was −66.3 (SD=97.81) and −116.3 (SD=102.88) and the proportion of subjects in clinical remission at Week 8 was 20% and 40% for the placebo and ustekinumab 6 mg/kg groups, respectively. These assumptions incorporated the impact of 4% of subjects being non-completers (in CNTO1275CRD3002).

Reflective of the availability of therapeutic options in the Bio-NF population, for Study 2, sample size estimations were based on a desired effect greater than that demonstrated by previously evaluated therapeutics (e.g., ustekinumab). Therefore, assuming the mean CDAI change from baseline at Week 8 is −152 in the anti-NKG2D antibody group (derived based on remission rate of 50%, which is 10% greater than the ustekinumab remission rate in CNTO1275CRD3002) and −66 in the placebo group with a common SD of 100, 50 subjects per treatment group will provide 99% power to detect a treatment difference between anti-NKG2D antibody and placebo at an overall Type 1 error of 0.05 (2-sided; Table).

Fifty Bio-NF subjects per treatment group will also provide 89% power to detect a difference from placebo in the proportion of subjects in clinical remission at Week 8 at an overall Type 1 error of 0.05 (2-sided; Table), assuming anti-NKG2D antibody has a remission rate of 50%, which is 10% greater than the ustekinumab remission rate in CNTO1275CRD3002.

### Bio-NF Subjects Who Are SNP-Positive (Bio-NF/SNP+)

Based on the assumption that 75% of the Crohn's disease population will be SNP-positive, 50 Bio-NF subjects will provide approximately 38 Bio-NF/SNP+ subjects. Thirty-eight Bio-NF/SNP+ subjects per group will provide 80% power to detect a difference from placebo in the proportion of subjects in clinical remission at Week 8 at an overall Type 1 error of 0.05 (2-sided; Table), assuming the anti-NKG2D antibody has a remission rate of 50%, which is 10% greater than the ustekinumab remission rate in CNTO1275CRD3002.

### Example 24

### Sample Size in Part II (Study 3)

### All Bio-IR Subjects

Using the same assumptions as were used for the Bio-IR population in Study 1, 50 subjects per treatment group will provide a mean power of 85% to detect a dose response signal for change from baseline in CDAI at Week 8 based on 7 candidate dose response models (to be detailed in the SAP) at an overall Type 1 error of 0.05 (2-sided). Fifty subjects per treatment group will also provide approximately 80% power to detect a treatment difference between the anti-NKG2D antibody treatment group with the highest dose and the placebo treatment group for change from baseline in CDAI at Week 8 at a Type I error of 0.05 (2-sided; Table). This will result in a total sample size of 250 subjects in Part II (incorporating an additional 50 subjects for the ustekinumab treatment group). Fifty Bio-IR subjects per treatment group in Part II will also provide 90% power to detect a difference between the anti-NKG2D antibody treatment group with the highest dose and the placebo treatment group in the proportion of subjects in clinical remission at Week 8 (the first major secondary endpoint) at a Type 1 error of 0.05 (2-sided; Table), assuming the anti-NKG2D antibody has a remission rate of 31%, which is 10% greater than the ustekinumab remission rate in CNTO1275CRD3001.

### Bio-IR Subjects Who Are SNP-Positive (Bio-IR/SNP+)

Based on the assumption that 75% of the Crohn's disease population will be SNP-positive, 50 Bio-IR subjects will provide approximately 38 Bio-IR/SNP+subjects. Thirty-eight Bio-IR/SNP+subjects per group will provide 80% power to detect a difference between the anti-NKG2D antibody treatment group with the highest dose and the placebo treatment group in the proportion of subjects in clinical remission at Week 8 at a Type 1 error of 0.05 (2-sided; Table), assuming the anti-NKG2D antibody has a remission rate of 31%, which is 10% greater than the ustekinumab remission rate in CNTO1275CRD3001.

### Efficacy Analyses

This protocol is comprised of 3 separate studies. Each study will be analyzed separately with separate Type I error control for the primary endpoint (at the 0.05 level of significance). The other endpoints within each study will not be controlled for multiplicity.

Three analysis sets, one for each study, will be used for the analyses planned in this protocol. For each study, the analysis set is all randomized subjects who received study agent. Efficacy analyses will be based on a modified intent-to-treat principle. Therefore, the efficacy data for each subject who received study agent will be analyzed according to the assigned treatment regardless of the actual treatment received.

### Example 25

### Study 1 (PART I Bio-IR Subjects) Primary Endpoint Analysis

The primary endpoint for the Bio-IR subjects in Part I is the change from baseline in the CDAI score at Week 8.

The change from baseline in the CDAI score will be compared between the anti-NKG2D antibody treatment group and the placebo treatment group. For the comparison, an ANCOVA model on the van der Waerden normal scores will be used with treatment as a fixed factor and baseline CDAI score and SNP-positive status (yes or no) as covariates. For this analysis, treatment failure rules and missing data rules as specified in Section 0 will be applied.

Study 1 will be considered to be a positive study if a significant improvement is detected in the change from baseline in the CDAI score at Week 8 in the anti-NKG2D antibody group compared with the placebo group at the 0.05 level of significance.

### Other Efficacy Endpoint Analyses

The following endpoints will be compared between the anti-NKG2D antibody treatment group and the placebo treatment group:
Change in CDAI from baseline at all postbaseline visits.
Clinical remission based on CDAI at all postbaseline visits.
Clinical response based on CDAI at all postbaseline visits.
Change in PRO-2 from baseline at all postbaseline visits.
Change in abdominal pain score (mean daily average based on the CDAI assessment) from baseline at all postbaseline visits.
Change in stool frequency score (mean daily average based on the CDAI assessment) from baseline at all postbaseline visits.
Clinical remission based on PRO-2 at all postbaseline visits.
Clinical response based on PRO-2 at all postbaseline visits.
Change in PRO-3 from baseline at all postbaseline visits.
Clinical remission based on CDAI at Week 24 among subject
s in clinical response at

Week 8.
Clinical remission based on CDAI at Week 24 among subjects in clinical remission at Week 8.
Change in SES-CD score from baseline at Weeks 12 and 24.
Endoscopic improvement at Weeks 12 and 24 based on a reduction from baseline in SES-CD score ≥3.
At least 50% improvement from baseline in SES-CD at Weeks 12 and 24.
Endoscopic healing (defined as the absence of mucosal ulcerations) at Weeks 12 and 24.
Fistula response at all postbaseline visits, defined as a ≥50% reduction from baseline in the number of draining fistulas.
Endpoint(s) based on Bristol stool form scale (to be detailed in the SAP).
Change in abdominal pain from baseline at all postbaseline visits based on a 0-10 NRS.
Change in IBDQ score from baseline at Weeks 8, 12, and 24.
Clinical remission based on IBDQ (≥170) at Weeks 8, 12, and 24.
A ≥16-point improvement in IBDQ from baseline at Weeks 8, 12, and 24.
Change from baseline in the PCS and MCS scores of the SF-36 at Weeks 8, 12, and 24.
A ≥5-point improvement in PCS or MCS scores of the SF-36 at Weeks 8, 12, and 24.
Change in biomarkers (CRP, fecal calprotectin, fecal lactoferrin) from baseline at Weeks 8, 12, and 24.
Clinical remission based on CDAI at Week 8 by SNP status. Subjects who are positive in at least 1 of 2 SNPs (NKG2D or MICB) will be considered to be SNP-positive. Other efficacy endpoints may be examined by SNP status (to be detailed in the SAP).

### Example 26

### Study 2 (PART I Bio-NF Subjects) Primary Endpoint Analysis

The primary endpoint for the Bio-NF subjects in Part I is the change from baseline in the CDAI score at Week 8.

The change from baseline in the CDAI score will be compared between the anti-NKG2D antibody treatment group and the placebo treatment group. For the comparison, an ANCOVA model on the van der Waerden normal scores will be used with treatment as a fixed factor and baseline CDAI score and SNP-positive status (yes or no) as covariates. For this analysis, treatment failure rules and missing data rules will be applied. Study 2 will be considered to be a positive study if a significant improvement is detected in the change from baseline in the CDAI score at Week 8 in the anti-NKG2D antibody group compared with the placebo group at the 0.05 level of significance.

### Other Efficacy Endpoint Analyses

Comparisons between the anti-NKG2D antibody treatment group and the placebo treatment group will also be made for each of the endpoints specified.

### Example 27

### Study 3 (PART II) Primary Endpoint Analysis

The primary endpoint is the change from baseline in the CDAI score at Week 8. A unified strategy that combines multiple comparison procedures with modeling techniques, MCP-Mod, will be used to analyze the dose-response relationship for the anti-NKG2D antibody doses (the efficacy measurement for the dose-response analysis is the change from baseline in the CDAI score at Week 8). This approach consists of 2 major steps. The first step consists of testing the dose-response signal via multiple contrast tests while controlling the overall Type 1 error. If a dose-response signal is detected, the second step is to select a model that best describes the observed data and use it to estimate adequate doses with associated precision. The details of the dose-response analysis will be provided in the SAP.

Study 3 will be considered positive if a dose response signal for the primary endpoint is detected. In addition to the dose-response analysis, pairwise comparisons of the anti-NKG2D antibody treatment groups versus the placebo group will be performed for the change from baseline in the CDAI score at Week 8; these comparisons will not be adjusted for multiplicity. For these comparisons, an ANCOVA model on the van der Waerden normal scores will be used with treatment as a fixed factor and baseline CDAI score and SNP-positive status (yes or no) as covariates. Pairwise comparisons of the ustekinumab treatment group with the anti-NKG2D antibody treatment groups or with placebo are not planned; however, summary statistics will be provided for the ustekinumab treatment group.

For the analyses described above, subjects who meet 1 or more treatment failure rules before Week 8 will have their baseline value for the CDAI score carried forward to Week 8. Subjects who have any of the following events before the Week 8 visit will be considered to be treatment failures for the primary endpoint analysis, regardless of the actual CDAI score:
Specified changes in concomitant Crohn's disease medications (to be detailed in the SAP).
A Crohn's disease-related surgery (with the exception of drainage of an abscess or seton placement).
Discontinuation of study agent due to lack of efficacy or due to an AE of worsening Crohn's disease.

In addition, subjects who do not return for evaluation or have insufficient data to calculate their CDAI score at Week 8 (i.e., <4 components of the CDAI are available) will have their last available CDAI score carried forward for Week 8.

To examine the robustness of the primary endpoint analysis, sensitivity analyses of the primary endpoint will be conducted using different missing data approaches; these analyses will be described in the SAP. In addition, sensitivity analyses excluding subjects who do not meet predefined threshold values of stool frequency and abdominal pain at study entry will be performed for the primary endpoint; the threshold values and analyses will also be described in the SAP.

Major Secondary Endpoint Analyses The major secondary endpoints are:
Clinical remission at Week 8 as measured by CDAI (CDAI <150).
Clinical response at Week 8 as measured by CDAI (≥100-point reduction from baseline in CDAI or CDAI <150).
Change in PRO-2 from baseline at Week 8.
Clinical remission at Week 8 as measured by PRO-2 (PRO-2 <75).
Clinical response at Week 8 as measured by PRO-2 (≥50-point reduction from baseline in PRO-2 or PRO-2 <75).
Change in SES-CD from baseline at Week 12.

The major secondary endpoints of clinical remission and clinical response at Week 8 (defined by either CDAI or PRO-2) will be compared between each of the anti-NKG2D antibody treatment groups and the placebo group using the Cochran-Mantel-Haenszel (CMH) chi-square test (2-sided) stratified by baseline CDAI score (≤300 or >300) and SNP-positive status (yes or no). In addition, for the endpoint of clinical remission at Week 8 as measured by CDAI, the MCP-MOD strategy will be used to examine the dose response relationship for the anti-NKG2D antibody doses.

Subjects who meet 1 or more treatment failure rules (as specified for the primary endpoint) before Week 8 will be considered not to be in clinical remission or clinical response. Subjects who have a missing CDAI score (i.e., <4 components of the CDAI score) at Week 8 will be considered not to be in clinical remission or clinical response, as measured by the CDAI score. Subjects who have a missing PRO-2 score (i.e., at least one component score of the PRO-2 is missing) at Week 8 will be considered not to be in clinical remission or clinical response as measured by the PRO-2 score.

The change in PRO-2 from baseline at Week 8 will be compared between each of the anti-NKG2D antibody treatment groups and the placebo group using an ANCOVA model on the van der Waerden normal scores with treatment as a fixed factor and baseline PRO-2 score and SNP-positive status (yes or no) as covariates.

Subjects who meet 1 or more treatment failure rules before Week 8 will have their baseline PRO-2 score carried forward to Week 8. Subjects who do not return for evaluation or who have a missing PRO-2 score at Week 8 will have their last available PRO-2 score carried forward to Week 8.

The change in SES-CD score from baseline at Week 12 will be compared between each of the anti-NKG2D antibody treatment groups and the placebo group using an ANCOVA model on the van der Waerden normal scores with treatment as a fixed factor and baseline SES-CD score and SNP-positive status (yes or no) as covariates. Data-handling rules for the SES-CD score will be provided in the SAP.

For the major secondary endpoints, pairwise comparisons of the ustekinumab treatment group with the anti-NKG2D antibody treatment groups or with placebo are not planned; however, summary statistics will be provided for the ustekinumab treatment group. Sensitivity analyses excluding subjects who do not meet predefined threshold values of stool frequency and abdominal pain at study entry will be performed for the major secondary endpoints; the threshold values and analyses will be described in the SAP. No adjustments for multiple comparisons will be made for the major secondary endpoints.

### Other Efficacy Endpoint Analyses

Comparisons between each of the anti-NKG2D antibody treatment groups and the placebo treatment group will also be made for each of the endpoints spe. Pairwise comparisons of the ustekinumab treatment group with the anti-NKG2D antibody treatment groups or with placebo are not planned for these endpoints, however summary statistics will be provided for the ustekinumab group.

### Pharmacokinetic Analyses

Descriptive statistics of the serum anti-NKG2D antibody and ustekinumab concentrations will be calculated at each sampling time point. Serum anti-NKG2D antibody and ustekinumab concentrations over time will be summarized for each treatment group.

Concentrations below the lowest quantifiable concentration will be treated as zero in the summary statistics.

A population PK analysis approach for anti-NKG2D antibody using nonlinear mixed-effects modeling (NONMEM) will be used to evaluate PK parameters. The influence of important covariates on the population PK parameter estimates may be evaluated. Details will be provided in a population PK analysis plan and the results of the population PK analysis will be presented in a separate technical report.

### Immunogenicity Analyses

The incidence and titers of antibodies to the anti-NKG2D antibody and antibodies to ustekinumab will be summarized for all subjects who receive a dose of the anti-NKG2D antibody or ustekinumab and have appropriate samples for detection of antibodies to the anti-NKG2D antibody or antibodies to ustekinumab (i.e., subjects with at least 1 sample obtained after their first dose of anti-NKG2D antibody or ustekinumab).

A listing of subjects who are positive for antibodies to anti-NKG2D antibody or ustekinumab will be provided. The maximum titers of antibodies to anti-NKG2D antibody or ustekinumab will be provided for subjects who are positive for antibodies to anti-NKG2D antibody or ustekinumab.

The incidence of neutralizing antibodies (NAbs) to anti-NKG2D antibody or ustekinumab will be summarized for subjects who are positive for antibodies to anti-NKG2D antibody or ustekinumab and have samples evaluable for NAbs to anti-NKG2D antibody or ustekinumab.

### Biomarker Analyses

Biomarker analyses will characterize the effects of the anti-NKG2D antibody on the measured biomarkers to identify biomarkers relevant to treatment and to determine if these biomarkers can predict response to the anti-NKG2D antibody. Biomarker analyses of ustekinumab will be performed as comparisons but not to identify novel biomarkers for ustekinumab.

Results of serum, whole blood analyses, stool, and mucosal biopsy analyses (including histology) will be reported in separate technical reports.

### Pharmacokinetic/Pharmacodynamic Analyses

The relationship between serum concentrations of anti-NKG2D antibody and PD and/or clinical endpoints will be examined.

NKG2D RO (%) over time will be summarized for each treatment group.

The absolute numbers and percentages of peripheral blood NK cells and T cells (including CD4+ and CD8+) over time will be summarized for each treatment group.

### Pharmacogenomic Analyses

Exploratory genetic analyses on DNA collection from subjects who signed the optional DNA consent will be presented in a separate technical report.

### Interim Analysis

An interim analysis is planned in Study 1 when the first 80% of the randomized Part I Bio-IR subjects (at least 40 Bio-IR subjects and at least 30 Bio-IR/SNP-positive subjects per treatment group) have completed their Week 8 visit or have terminated their study participation before Week 8. This interim analysis will allow for an earlier start of Part II (i.e., Study 3, the dose-ranging part) if the results suggest that a sufficient number of subjects have been evaluated for the purpose of demonstrating effect. As this interim analysis does not affect the conduct or completion of Study 1, it will be considered administrative and will not require multiplicity adjustment for the final Study 1 analysis.

The primary efficacy evaluation is the comparison between the anti-NKG2D antibody and placebo with respect to clinical remission at Week 8 (in Bio-IR subjects), as remission is a more stringent endpoint than change in CDAI (the primary endpoint for this study) and provides a more conservative decision rule to determine whether to start Part II early. Other selected efficacy analyses (e.g., change in CDAI and PRO-2, clinical response) will also been performed; details will be provided in the Interim Analysis Plan.

A sponsor committee independent of the study team will be established to review the interim data and formulate recommended decisions/actions in accordance with predefined decision rules that will be defined in the Interim Analysis Plan.

### Example 28

### Study Drug Information

### Physical Description of Study Drugs

In Part I and Part II, the anti-NKG2D antibody supplied for this study is a lyophilized drug product which, upon reconstitution with 1.1 mL of water for injection, contains 100 mg/mL anti-NKG2D antibody in 34 mM L-histidine, 8.6% (w/v) sucrose, and 0.03% (w/v) polysorbate 80, pH 6.0 in a 10 mL glass vial. It will be manufactured and provided under the responsibility of the sponsor.

Placebo for the anti-NKG2D antibody consists of a 9 mL solution of 34 mM L-histidine, 8.6% (w/v) sucrose, and 0.03% (w/v) polysorbate 80, pH 6.0 in a 10 mL glass vial.

In Part II, ustekinumab 5 mg/mL final vialed product for IV infusion and placebo to match will be supplied as a single-use, sterile solution in 30 mL vials with 1 dose strength (i.e., 130 mg in 26 mL nominal volume).

Ustekinumab SC will be supplied as a sterile solution in a single-use prefilled syringe (PFS) at a volume of 1 mL (90 mg dose) that contains ustekinumab 90 mg, L-histidine, L-histidine monohydrochloride monohydrate, sucrose, and polysorbate 80 at pH 6.0 in 1.0 mL nominal volume. No preservatives are present. The needle cover on the PFS contains dry natural rubber (a derivative of latex), which may cause allergic reactions in individuals sensitive to latex. Liquid placebo will be supplied in a 1 mL PFS.

Placebo administrations will have the same appearance as the respective the anti-NKG2D antibody or ustekinumab administrations.

What is claimed is:

1. A method of treating a human patient with Crohn's disease, the method comprising administering to the human patient a safe and effective amount of an anti-NKG2D antibody comprising CDR1, CDR2 and CDR3 domains of the heavy chain variable region having the sequences set forth in SEQ ID NO: 3, 4 and 5, respectively and CDR1, CDR2 and CDR3 domains of the light chain variable region having the sequences set forth in SEQ ID NO: 6, 7, and 8, respectively, wherein the method comprises at least one administration cycle, wherein for each of the at least one cycle, the anti-NKG2D antibody is administered as follows: (a) one dose of 400 mg anti-NKG2D antibody and (b) at least one dose of 200 mg anti-NKG2D antibody.

2. The method of claim 1, wherein the anti-NKG2D antibody is formulated for intravenous or subcutaneous administration.

3. The method of claim 1, wherein the treatment consists of up to 6 cycles.

4. The method of claim 1, wherein the 200 mg dose is administered eleven times.

5. The method of claim 1, wherein the administration of dose (b) of the NKG2D antibody is administered once every 2 weeks for 22 weeks.

6. The method of claim 1, wherein the anti-NKG2D antibody produces an effect in the human patient selected from the group consisting of reduction of a symptom of Crohn's disease, induction of a clinical response, induction or maintenance of clinical remission, inhibition of disease progression and inhibition of a disease complication.

7. The method of claim 1, wherein the anti-NKG2D antibody produces an effect in the human patient selected from the group consisting of reduction of a Crohn's Disease Activity Index score, decrease in C-Reactive Protein expression level, decrease in fecal calprotein expression level and reduction in the number of open draining fistulas.

8. The method of claim 7, wherein the Crohn's Disease Activity Index score of the human patient is reduced by at least 100 points.

9. The method of claim 7, wherein the Crohn's Disease Activity Index score of the human patient is reduced to less than 150.

10. The method of claim 6, wherein the reduction in a symptom of Crohn's disease is a decrease of at least 50% of open draining fistulas in the human patient.

11. The method of claim 1, wherein the human patient has been diagnosed with active moderate to severe Crohn's disease prior to the administration of the anti-NKG2D antibody.

12. The method of claim 1, wherein the human patient has a Crohn's Disease Activity Index score of 220-450 prior to the administration of the anti-NKG2D antibody.

13. The method of any one of claims 1-12, wherein the human patient has a genotype comprising a single nucleotide polymorphism (SNP) rs2255336 and/or rs2239705 prior to the administration of the anti-NKG2D antibody.

14. The method of claim 1, wherein the anti-NKG2D antibody comprises a heavy-chain variable region comprising SEQ ID NO: 1 and a light-chain variable region comprising SEQ ID NO: 2.

15. A method of treating a human patient with Crohn's disease, the method comprising the steps of:
(a) determining whether the human patient has a SNP in an NKG2D receptor gene or MICB gene by obtaining a biological sample from the human patient and performing a genotyping assay on the biological sample;
(b) administering an anti-NKG2D receptor antibody if the patient has the SNP, wherein the anti-NKG2D antibody comprises CDR1, CDR2 and CDR3 domains of the heavy chain variable region having the sequences set forth in SEQ ID NO: 3, 4 and 5, respectively and CDR1, CDR2 and CDR3 domains of the light chain variable region having the sequences set forth in SEQ ID NO: 6, 7, and 8, respectively.

16. The method of claim 15, wherein the anti-NKGD2 antibody is administered in at least one cycle comprising the administration of (a) one dose of 400 mg anti-NKG2D antibody and (b) at least one dose of 200 mg anti-NKG2D antibody.

17. The method of claim 15, wherein the SNP is rs2255336 or rs2239705.
